(19) United States
(12) Patent Application Publication
(10) Pub. No.: US 2007/0254889 A1
(43) Pub. Date: Nov. 1, 2007

(54) Compounds and methods useful for rescuing cells from beta-amyloid toxicity and treatment of Alzheimer's disease

(76) Inventors: Lee-Way Jin, Davis, CA (US); Hank F. Kung, Wynnewood, PA (US); Mei-Ping Kung, Wynnewood, PA (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/600,280

(22) Filed: Nov. 16, 2006

Related U.S. Application Data
(63) Continuation-in-part of application No. 10/127,678, filed on Apr. 23, 2002, now Pat. No. 6,696,039.
(63) Continuation-in-part of application No. 10/739,217, filed on Dec. 19, 2003, now Pat. No. 6,946,116.
(63) Continuation-in-part of application No. 11/203,429, filed on Aug. 15, 2005.
(63) Continuation-in-part of application No. 10/228,275, filed on Aug. 27, 2002, now Pat. No. 7,250,525.
(63) Continuation-in-part of application No. 11/218,587, filed on Sep. 6, 2005.
(63) Continuation-in-part of application No. PCT/US03/31466, filed on Oct. 6, 2003.
(63) Continuation-in-part of application No. 10/529,850, filed on Mar. 31, 2005.
(60) Provisional application No. 60/832,752, filed on Jul. 24, 2006.
(60) Provisional application No. 60/285,282, filed on Apr. 23, 2001.
(60) Provisional application No. 60/415,824, filed on Oct. 4, 2002.

(51) Int. Cl. A61K 31/437; A61K 31/416; A61K 31/4184; A61K 31/495; A61P 25/28; A61K 31/423; A61K 31/428

(52) U.S. Cl. 514/252.05; 514/254.04; 514/258.1; 514/300; 514/367; 514/375; 514/394; 514/415; 514/469; 514/646; 514/680; 514/753

(57) ABSTRACT

The present invention is directed to pharmaceutical compositions comprising one or more compounds of Formulae I, II, III, IV, V, VI, VII, VIII, IX and X, or pharmaceutically acceptable salts thereof and excipients. The present invention provides a method of inhibiting β-amyloid plaque aggregation, the method comprising introducing into a mammal an aggregation-inhibiting amount of a compound of Formula I, II, III, IV, V, VI, VII, VIII, IX or X or a pharmaceutically acceptable salt, ester, amide or prodrug thereof. By inhibiting amyloid aggregation, this method is capable of rescuing cells that otherwise would be susceptible or further damaged by amyloidosis.

### CROSS REFERENCE TO RELATED APPLICATIONS

This application claims the benefit of U.S. Provisional Application No. 60/832,752, filed Jul. 24, 2006; and is a continuation-in-part of U.S. patent application Ser. No. 10/127,678, filed Apr. 23, 2002, now U.S. Pat. No. 6,696,039, U.S. patent application Ser. No. 10/739,217, filed Dec. 19, 2003, now U.S. Pat. No. 6,946,116, and U.S. patent application Ser. No. 11/203,429, filed Aug. 15, 2005, each of which claims the benefit of U.S. Provisional Application No. 60/285,282, filed Apr. 23, 2001; a continuation-in-part of U.S. patent application Ser. No. 10/228,275, filed Aug. 27, 2002, and Ser. No. 11/218,587, filed Sep. 6, 2005, each of which claims the benefit of U.S. Provisional Application No. 60/314,658, filed Aug. 27, 2001; and a continuation-in-part of International Application No. PCT/US2003/031466 and U.S. patent application Ser. No. 10/529,850, filed Mar. 31, 2005, each of which claims the benefit of U.S. Provisional Application No. 60/415,824, filed Oct. 4, 2002; the contents of these applications are entirely incorporated by reference herein.

### BACKGROUND OF THE INVENTION

1. Field of the Invention

This invention relates to methods of rescuing cells from amyloid-associated toxicity, and methods of inhibiting amyloid aggregation.

2. Background Art

Alzheimer's disease (AD) is a progressive neurodegenerative disorder characterized by cognitive decline, irreversible memory loss, disorientation, and language impairment. Postmortem examination of AD brain sections reveals abundant senile plaques (SPs) composed of amyloid-β (Aβ) peptides and numerous neurofibrillary tangles (NFTs) formed by filaments of highly phosphorylated tau proteins (for recent reviews and additional citations see Ginsberg, S. D., et al., “Molecular Pathology of Alzheimer's Disease and Related Disorders,” in Cerebral Cortex: Neurodegenerative and Age-related Changes in Structure and Function of Cerebral Cortex, Kluwer Academic/Plenum, NY (1999), pp. 603-654; Vogelsberg-Ragaglia, V., et al., “Cell Biology of Tau and Cytoskeletal Pathology in Alzheimer's Disease,” Alzheimer's Disease, Lippincot, Williams & Wilkins, Philadelphia, Pa. (1999), pp. 359-372). Familial AD (FAD) is caused by multiple mutations in the A precursor protein (APP), presenilin 1 (PS1) and presenilin 2 (PS2) genes (Ginsberg, S. D., et al., “Molecular Pathology of Alzheimer's Disease and Related Disorders,” in Cerebral Cortex: Neurodegenerative and Age-related Changes in Structure and Function of Cerebral Cortex, Kluwer Academic/Plenum, NY (1999), pp. 603-654; Vogelsberg-Ragaglia, V., et al., “Cell Biology of Tau and Cytoskeletal Pathology in Alzheimer's Disease,” Alzheimer's Disease, Lippincot, Williams & Wilkins, Philadelphia, Pa. (1999), pp. 359-372).

While the exact mechanisms underlying AD are not fully understood, all pathogenic FAD mutations studied thus far increase production of the more amyloidogenic 42-43 amino-acid long form of the Aβ peptide. Thus, at least in FAD, dysregulation of Aβ production appears to be sufficient to induce a cascade of events leading to neurodegeneration. Indeed, the amyloid cascade hypothesis suggests that formation of extracellular fibrillar Aβ aggregates in the brain may be a pivotal event in AD pathogenesis (Selkoe, D. J., “Biology of β-amyloid Precursor Protein and the Mechanism of Alzheimer's Disease,” Alzheimer's Disease, Lippincot Williams & Wilkins, Philadelphia, Pa. (1999), pp. 293-310; Selkoe, D. J., J. Am. Med. Assoc. 283:1615-1617 (2000); Naslund, J., et al., J. Am. Med. Assoc. 283:1571-1577 (2000); Golde, T. E., et al., Biochimica et Biophysica Acta 1502:172-187 (2000)).

Various approaches in trying to inhibit the production and reduce the accumulation of fibrillar Aβ in the brain are currently being evaluated as potential therapies for AD (Skovronsky, D. M. and Lee, V. M., Trends Pharmacol. Sci. 21:161-163 (2000); Vassar, R., et al., Science 286:735-741 (1999); Wolfe, M. S., et al., J. Med. Chem. 41:6-9 (1998); Moore, C. L., et al., J. Med. Chem. 43:3434-3442 (2000); Findeis, M. A., Biochimica et Biophysica Acta 1502:76-84 (2000); Kuner, P., Bohrmann, et al., J. Biol. Chem. 275:1673-1678 (2000)). It is therefore of great interest to develop ligands that specifically bind fibrillar Aβ aggregates. Since extracellular SPs are accessible targets, these new ligands could be used as in vivo diagnostic tools and as probes to visualize the progressive deposition of Aβ in studies of AD amyloidogenesis in living patients.

To this end, several interesting approaches for developing fibrillar Aβ aggregate-specific ligands have been reported (Ashburn, T. T., et al., Chem. Biol. 3:351-358 (1996); Han, G., et al., J. Am. Chem. Soc. 118:4506-4507 (1996); Klunk, W. E., et al., Biol. Psychiatry 35:627 (1994); Klunk, W. E., et al., Neurobiol. Aging 16:541-548 (1995); Klunk, W. E., et al., Society for Neuroscience Abstract 23:1638 (1997); Mathis, C. A., et al., Proc. XIIth Intl. Symp. Radiopharm. Chem., Uppsala, Sweden: 94-95 (1997); Lorenzo, A. and Yankner, B. A., Proc. Natl. Acad. Sci. U.S.A. 91:12243-12247 (1994); Zhen, W., et al., J. Med. Chem. 42:2805-2815 (1999)). The most attractive approach is based on highly conjugated chrysamine-G (CG) and Congo red (CR), and the latter has been used for fluorescent staining of SPs and NFTs in postmortem AD brain sections (Ashburn, T. T., et al., Chem. Biol. 3:351-358 (1996); Klunk. W. E., et al., J. Histochem. Cytochem. 37:1273-1281 (1989)). The inhibition constants (Kᵢ) for binding to fibrillar Aβ aggregates of CR, CG, and 3′-bromo- and 3′-iodo derivatives of CG are 2,800, 370, 300 and 250 nM, respectively (Mathis, C. A., et al., Proc. XIIth Intl. Symp. Radiopharm. Chem., Uppsala, Sweden: 94-95 (1997)). These compounds have been shown to bind selectively to Aβ (1-40) peptide aggregates in vitro as well as to fibrillar Aβ deposits in AD brain sections (Mathis, C. A., et al., Proc. XIIth Intl. Symp. Radiopharm. Chem., Uppsala, Sweden: 94-95 (1997)).

Amyloidosis is a condition characterized by the accumulation of various insoluble, fibrillar proteins in the tissues of a patient. An amyloid deposit is formed by the aggregation of amyloid proteins, followed by the further combination of aggregates and/or amyloid proteins. Formation and accumulation of aggregates of β-amyloid (Aβ) peptides in the brain are critical factors in the development and progression of AD.

The fibrillar aggregates of amyloid peptides, Aβ₁₋₄₀ and Aβ₁₋₄₂, are major metabolic peptides derived from amyloid precursor protein found in senile plaques and cerebrovascular amyloid deposits in AD patients (Xia, W., et al., J. Proc. Natl. Acad. Sci. U.S.A. 97:9299-9304 (2000)). Prevention and reversal of Aβ plaque formation are being targeted as a treatment for this disease (Selkoe, D., J. JAMA 283:1615-1617 (2000); Wolfe, M. S., et al., J. Med. Chem. 41:6-9 (1998); Skovronsky, D. M., and Lee, V. M., Trends Pharmacol. Sci. 21:161-163 (2000)).

In addition to the role of amyloid deposits in Alzheimer's disease, the presence of amyloid deposits has been shown in diseases such as Mediterranean fever, Muckle-Wells syndrome, idiopathetic myeloma, amyloid polyneuropathy, amyloid cardiomyopathy, systemic senile amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, Isolated atrial amyloid, β₂-microglobulin amyloid in dialysis patients, inclusion body myositis, β₂-amyloid deposits in muscle wasting disease, and Islets of Langerhans diabetes Type II insulinoma.

Potential compounds for the inhibition of Aβ aggregates and the treatment of AD in the living brain must cross the intact blood-brain barrier. Thus brain uptake can be improved by using ligands with relatively smaller molecular size (compared to Congo Red) and increased lipophilicity. Highly conjugated thioflavins (S and T) are commonly used as dyes for staining the Aβ aggregates in the AD brain (Elhaddaoui, A., et al., Biospectroscopy 1: 351-356 (1995)). These compounds are based on benzothiazole, which is relatively small in molecular size.

It would be useful to have compounds and methods of rescuing cells from amyloid-associated toxicity and treating AD. Such compounds and methods may also be useful for inhibiting amyloid aggregation.

### SUMMARY OF THE INVENTION

The present invention is directed to pharmaceutical compositions comprising one or more compounds of Formulae I, II, III, IV, V, VI, VII, VIII, IX and X, or pharmaceutically acceptable salts thereof and excipients. The present invention provides a method of inhibiting β-amyloid plaque aggregation, the method comprising introducing into a mammal an aggregation-inhibiting amount of a compound of Formula I, II, III, IV, V, VI, VII, VIII, IX or X or a pharmaceutically acceptable salt, ester, amide or prodrug thereof. By inhibiting amyloid aggregation, this method is capable of rescuing cells that otherwise would be susceptible or further damaged by amyloidosis.

### BRIEF DESCRIPTION OF THE FIGURES

FIG. 1 depicts representative compounds of the present invention and the binding data for these compounds.

FIG. 2 depicts representative compounds of the present invention and the binding data for these compounds.

FIGS. 3-19 depict cell viability after treatment with compounds of the present invention as measured by MTT assay. The figures depict the rescuing effect or protection by measuring cell survival (viability) as compared between cells with amyloid aggregation “Tet(−)” and cells without amyloid aggregation “Tet(+).” The cell survival of the Tet(+) cells thus merely reflects the toxicity of the drug, and serves as a control. A rescuing effect was demonstrated by a cell survival % of Tet(−) cells increasingly near the cell survival % of Tet(+) cells at low concentrations that were not toxic to the cells.

### DETAILED DESCRIPTION OF THE INVENTION

One aspect of the present invention is directed to pharmaceutical compositions comprising one or more compounds of Formula I: 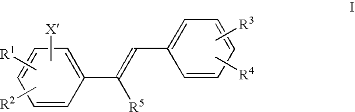
or a pharmaceutically acceptable salt thereof, wherein:

R⁵ is hydrogen or C₁₋₄ alkyl;

R¹, R² and R³, in each instance, is independently selected from the group consisting of hydrogen, hydroxy, halogen, C₁₋₄ alkyl, C₁₋₄ alkyloxy, cyano, carboxy(C₁₋₅)alkyl, trifluoromethyl, nitro, methylamino, dimethylamino, halo(C₁₋₄)alkyl, and formyl;

R⁴ is selected from the group consisting of:
a. C₁₋₄ alkylthio,
b. C₁₋₄ alkylsulfonyl,
c. hydroxy,
d. C₁₋₄ alkoxy,
e. NR⁶R⁷, wherein
   R⁶ and R⁷ are hydrogen or C₁₋₄ alkyl,
f. phenyl(C₁₋₄)alkyl,
g. C₆₋₁₀ aryl,
h. heteroaryl,
i. heterocycle,
j. heterocycle(C₁₋₄)alkyl, and
k. C₃₋₆ cycloalkyl,
   wherein said phenyl(C₁₋₄)alkyl, C₆₋₁₀ aryl, heteroaryl, heterocycle, heterocycle(C₁₋₄)alkyl or C₃₋₆ cycloalkyl is substituted with one of the following: C₁₋₄ alkylthio, C₁₋₄ alkyl sulfonyl, methoxy, hydroxy, dimethylamino or methylamino;

or R³ and R⁴ are taken together to form an optionally substituted aryl or heteroaryl ring, wherein said ring is attached at adjacent carbons on the appropriate stilbene ring;

and,

X′ is selected from the group consisting of hydrogen, halogen, halo(C₁₋₄)alkyl, halo(C₁₋₄)alkyl amino, halo(C₁₋₄)alkyl(C₁₋₄ alkyl)amino, and Sn(alkyl)₃.

The above description of compounds of Formula I include the following provisos, wherein in each instance, the halogen is other than a radiolabeled halogen, and if R⁴ is other than NR⁶R⁷, then R¹ is methylamino or dimethylamino.

Useful compounds falling within the scope of Formula I include compounds wherein R⁵ is hydrogen or C₁₋₄ alkyl. Especially useful values of R⁵ are hydrogen and methyl. The most useful value of R⁵ is hydrogen.

Useful compounds are those of Formula I wherein R¹, R² and R³, in each instance, is independently selected from the group as described above. In a preferred set of compounds, R¹, R² and R³ are selected from the group consisting of hydrogen, halogen, C₁₋₄ alkyl, cyano, carboxy(C₁₋₅)alkyl, trifluoromethyl, nitro, methylamino, dimethylamino, halo(C₁₋₄)alkyl, and formyl. In another preferred embodiment, R³ is hydrogen. In this preferred embodiment, it is especially preferred that R¹ and R² are independently selected from the group consisting of hydrogen, hydroxy, C₁₋₄ alkoxy, trifluoromethyl and C₁₋₄ alkyl. More preferably, at least one of R¹ and R² is hydrogen. Most preferably, R¹ is hydrogen and R² is hydroxy, trifluoromethyl or C₁₋₄ alkoxy.

Useful compounds of Formula I also include those compounds wherein R⁴ is as described above. Preferable values of R⁴ under the scope of C₆₋₁₀ aryl include phenyl, naphthyl or tetrahydronaphthyl. Preferable values of R⁴ under the scope of heteroaryl include thienyl, furyl, pyranyl, pyrrolyl, pyridinyl, indolyl, and imidazolyl. Preferable values of R⁴ under the scope of heterocycle include piperidinyl, pyrrolidinyl, and morpholinyl. In compounds wherein R⁴ is a preferred embodiment of a C₆₋₁₀ aryl, heteroaryl, heterocycle, heterocycle(C₁₋₄)alkyl or C₃₋₆ cycloalkyl, it is most preferable that the ring is substituted with one of the following: C₁₋₄ alkylthio, C₁₋₄ alkyl sulfonyl, methoxy, hydroxy, dimethylamino or methylamino. In another embodiment, R⁴ is more preferably selected from the group consisting of C₁₋₄ alkylthio, C₁₋₄ alkylsulfonyl, hydroxy, C₁₋₄ alkoxy, and NR⁶R⁷, wherein R⁶ and R⁷ are independently hydrogen or C₁₋₄ alkyl. Most preferably, R⁴ is selected from the group consisting of methylthio, methylsulfonyl, hydroxy, methoxy, or NR⁶R⁷, wherein R⁶ and R⁷ are independently hydrogen or methyl.

A preferred value of R³ and R⁴ includes an aromatic ring formed with both R³ and R⁴, wherein the ring is joined to the appropriate stilbene ring at adjacent carbons. This ring is preferably substituted with halogen, C₁₋₄ alkyl, hydroxy, amino, methylamino or dimethylamino.

Useful values of X′ include those already listed above. Preferably X′ is hydrogen or halogen. In another preferred embodiment, X′ is a non-radiolabeled halogen or Sn(alkyl)₃ especially in those compounds where R¹, R² and R³ are selected from the group consisting of hydrogen, halogen, C₁₋₄ alkyl, cyano, carboxy(C₁₋₅)alkyl, trifluoromethyl, nitro, methylamino, dimethylamino, halo(C₁₋₄)alkyl, and formyl, and R⁴ is mono or dialkyl amino. More preferably, R² is hydrogen and R³ is selected from the group consisting of hydrogen, hydroxy, C₁₋₄ alkyl, C₁₋₄ alkoxy and trifluoromethyl.

Another aspect of the present invention is directed to pharmaceutical compositions comprising one or more compounds of Formula II: 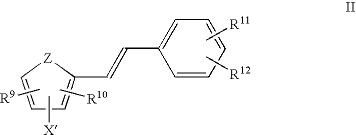
or a pharmaceutically acceptable salt thereof,

Z is O, S or NR^{a}, wherein
R^{a} is C₁₋₄ alkyl;

R⁹, R¹⁰ and R¹¹, in each instance, is independently selected from the group consisting of hydrogen, halogen, C₁₋₄ alkyl, cyano, carboxy(C₁₋₅)alkyl, trifluoromethyl, nitro, methylamino, dimethylamino, halo(C₁₋₄)alkyl, and formyl;

R¹² is selected from the group consisting of:
a. C₁₋₄ alkylthio,
b. C₁₋₄ alkylsulfonyl,
c. hydroxy,
d. C₁₋₄alkoxy,
e. NR¹³R¹⁴, wherein
   R¹³ and R¹⁴ are hydrogen or C₁₋₄ alkyl,
f. phenyl(C₁₋₄)alkyl,
g. C₆₋₁₀ aryl,
h. heteroaryl,
i. heterocycle,
j. heterocycle(C₁₋₄)alkyl, and
k. C₃₋₆ cycloalkyl,
   wherein said phenyl(C₁₋₄)alkyl, C₆₋₁₀ aryl, heteroaryl, heterocycle, heterocycle(C₁₋₄)alkyl or C₃₋₆ cycloalkyl is substituted with one of the following: C₁₋₄ alkylthio, C₁₋₄ alkyl sulfonyl, methoxy, hydroxy, dimethylamino or methylamino;

or R¹¹ and R¹² are taken together to form an optionally substituted aryl or heteroaryl ring, wherein said ring is attached at adjacent carbons on the appropriate stilbene ring;

and,

X′ is selected from the group consisting of hydrogen, halogen, halo(C₁₋₄)alkyl, halo(C₁₋₄)alkyl amino, halo(C₁₋₄)alkyl(C₁₋₄ alkyl)amino, and Sn(alkyl)₃. Preferably, X′ is halogen.

The above description of compounds of Formula II include the following provisos, wherein in each instance, the halogen is other than a radiolabeled halogen, and if R¹² is other than NR¹³R¹⁴, then R⁹ is methylamino or dimethylamino.

Useful compounds falling within the scope of Formula II include compounds wherein Z is O, S or NR^{a}, wherein R^{a} is C₁₋₄ alkyl. Especially useful compounds are those wherein Z is O.

Useful compounds are those of Formula II wherein R⁹, R¹⁰ and R¹¹, in each instance, is independently selected from the group as described above. Preferably, R¹¹ is hydrogen. In this preferred embodiment, it is especially preferred that R⁹ and R¹⁰ are independently selected from the group consisting of hydrogen and C₁₋₄ alkyl. More preferably, at least one of R⁹ and R¹⁰ is hydrogen. Most preferably, R⁹ and R¹⁰ are hydrogen.

Useful compounds of Formula II also include those compounds wherein R¹² is as described above. Preferable values of R¹² under the scope of C₆₋₁₀ aryl include phenyl, naphthyl or tetrahydronaphthyl. Preferable values of R¹² under the scope of heteroaryl include thienyl, furyl, pyranyl, pyrrolyl, pyridinyl, indolyl, and imidazolyl. Preferable values of R¹² under the scope of heterocycle include piperidinyl, pyrrolidinyl, and morpholinyl. In compounds wherein R¹² is a preferred embodiment of a C₆₋₁₀ aryl, heteroaryl, heterocycle, heterocycle(C₁₋₄)alkyl or C₃₋₆ cycloalkyl, it is most preferable that the ring is substituted with one of the following: C₁₋₄ alkylthio, C₁₋₄ alkyl sulfonyl, methoxy, hydroxy, dimethylamino or methylamino. In another embodiment, R¹² is more preferably selected from the group consisting of C₁₋₄ alkylthio, C₁₋₄ alkylsulfonyl, hydroxy, C₁₋₄ alkoxy, and NR¹³R¹⁴, wherein R¹³ and R¹⁴ are independently hydrogen or C₁₋₄ alkyl. Most preferably, R¹² is selected from the group consisting of methylthio, methylsulfonyl, hydroxy, methoxy, or NR¹³R¹⁴, wherein R¹³ and R¹⁴ are independently hydrogen or methyl.

Another aspect of the present invention is directed to pharmaceutical compositions comprising one or more compounds of Formula III: 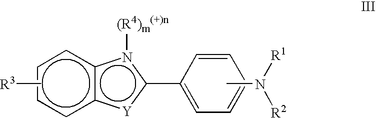
or a pharmaceutically acceptable salt thereof,
wherein:

Y is CH, NR⁵, O, S or CH═N, where R⁵ is hydrogen or a C₁₋₄ alkyl;

m and n are both zero, or m and n are both 1;

R³ is selected from the group consisting of —CH₃, hydrogen, halogen, halo(C₁₋₄)alkyl, halo(C₁₋₄)alkyl amino, halo(C₁₋₄)alkyl(C₁₋₄ alkyl)amino and Sn(alkyl)₃;

R¹ and R² are independently hydrogen, C₁₋₄ alkyl, C₂₋₄ aminoalkyl, C₁₋₄ haloalkyl, haloarylalkyl, or R¹ and R² are taken together with the nitrogen to which they are attached to form a 5- to 7-member heterocyclic ring optionally having O, S or NR⁶ in said ring, where

R⁶ is hydrogen or C₁₋₄ alkyl; and

R⁴ is C₁₋₄ alkyl.

The above description of compounds of Formula III include the following proviso, wherein in each instance, the halogen is other than a radiolabeled halogen.

A preferred group of compounds falling within the scope of the present invention include compounds of Formula III wherein Y is selected from NR⁵, O or S. Especially preferred compounds of Formula III include compounds wherein Y is NR⁵ or S, most preferably Y is O.

Preferred values of R⁵ in compounds of Formula III where Y is NR⁵ are hydrogen and C₁₋₄ alkyl, more preferably R⁵ is hydrogen or methyl, and most preferably R⁵ is hydrogen.

A preferred value of m and n in compounds of Formula III is from zero to one, more preferably zero.

Suitable values of R³ include those already listed above. Preferably R³ is halogen, C₁₋₄ alkyl, hydrogen or Sn(alkyl)₃.

Preferred compounds are those of Formula I wherein R¹ and R² are independently one of hydrogen, C₁₋₄ alkyl, C₁₋₄ haloalkyl, halophenyl(C₁₋₄)alkyl, or are taken together with the nitrogen to which they are attached to form a 5- to 7-member heterocyclic ring optionally having O or NR⁶ in said ring, where R⁶ is hydrogen or C₁₋₄ alkyl. Useful values of R¹ and R² include, independently, hydrogen, methyl, ethyl, propyl, isopropyl, butyl, t-butyl, isobutyl, 3-fluoropropyl, 4-fluorobutyl, or 4-fluorobenzyl, or R¹ and R² are taken together with the nitrogen to which they are attached to form a piperidinyl ring having NR⁶ in said ring, where R⁶ is hydrogen or methyl. More preferably, R¹ and R² are independently hydrogen or methyl.

Another aspect of the present invention is directed to pharmaceutical compositions comprising one or more compounds of Formula IV: 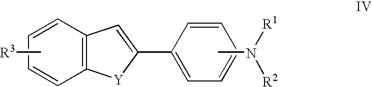
or a pharmaceutically acceptable salt thereof, wherein:
Y is O or NR⁴ where R⁴ is hydrogen or C₁₋₄alkyl;

R³ is selected from the group consisting of —CH₃, hydrogen, halogen, halo(C₁₋₄)alkyl, halo(C₁₋₄)alkyl amino, halo(C₁₋₄)alkyl(C₁₋₄ alkyl)amino and Sn(alkyl)₃;

R¹ and R² are independently hydrogen, C₁₋₄ alkyl, C₂₋₄ aminoalkyl, C₁₋₄ haloalkyl, haloarylalkyl, or R¹ and R² are taken together with the nitrogen to which they are attached to form a 5- to 7-member heterocyclic ring optionally having O, S or NR⁵ in said ring, where R⁵ is hydrogen or C₁₋₄ alkyl. More preferably, R¹ and R² are independently hydrogen or methyl.

The above description of compounds of Formula IV include the following proviso, wherein in each instance, the halogen is other than a radiolabeled halogen.

A preferred group of compounds include compounds of Formula IV where Y is NR⁴ where R⁴ is hydrogen or methyl. More preferred compounds include compounds where Y is O.

Useful values of R³ include those already listed above. More preferably, R³ is halogen, C₁₋₄ alkyl, hydrogen or Sn(alkyl)₃.

Preferred compounds are those of Formula IV wherein R¹ and R² are independently one of hydrogen, C₁₋₄ alkyl, C₁₋₄ haloalkyl, halophenyl(C₁₋₄)alkyl, or are taken together with the nitrogen to which they are attached to form a 5- to 7-member heterocyclic ring optionally having O or NR⁶ in said ring, where R⁶ is hydrogen or C₁₋₄ alkyl. Useful values of R¹ and R² include, independently, hydrogen, methyl, ethyl, propyl, butyl, t-butyl, isobutyl, 3-fluoropropyl, 4-fluorobutyl, or 4-fluorobenzyl, or R¹ and R² are taken together with the nitrogen to which they are attached to form a piperidinyl ring having NR⁶ in said ring, where R⁶ is hydrogen or methyl.

Another aspect of the present invention is directed to pharmaceutical compositions comprising one or more compounds of Formula V: 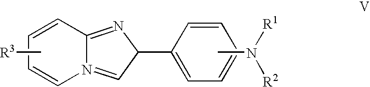
or a pharmaceutically acceptable salt thereof,
wherein:

R³ is selected from the group consisting of —CH₃, hydrogen, halogen, halo(C₁₋₄)alkyl, halo(C₁₋₄)alkyl amino, halo(C₁₋₄)alkyl(C₁₋₄ alkyl)amino and Sn(alkyl)₃;

R¹ and R² are independently hydrogen, C₁₋₄ alkyl, C₂₋₄ aminoalkyl, C₁₋₄ haloalkyl, haloarylalkyl, or R¹ and R² are taken together with the nitrogen to which they are attached to form a 5- to 7-member heterocyclic ring optionally having O, S or NR⁵ in said ring, where R⁵ is hydrogen or C₁₋₄ alkyl. More preferably, R¹ and R² are independently hydrogen or methyl.

The above description of compounds of Formula V include the following proviso, wherein in each instance, the halogen is other than a radiolabeled halogen.

Useful values of R³ include those already listed above. More preferably, R³ is halogen, C₁₋₄ alkyl, hydrogen or Sn(alkyl)₃.

Preferred compounds are those of Formula V wherein R¹ and R² are independently one of hydrogen, C₁₋₄ alkyl, C₁₋₄ haloalkyl, halophenyl(C₁₋₄)alkyl, or are taken together with the nitrogen to which they are attached to form a 5- to 7-member heterocyclic ring optionally having O or NR⁶ in said ring, where R⁶ is hydrogen or C₁₋₄ alkyl. Useful values of R¹ and R² include, independently, hydrogen, methyl, ethyl, propyl, butyl, t-butyl, isobutyl, 3-fluoropropyl, 4-fluorobutyl, or 4-fluorobenzyl, or R¹ and R² are taken together with the nitrogen to which they are attached to form a piperidinyl ring having NR⁶ in said ring, where R⁶ is hydrogen or methyl. Most preferably R¹ and R² are methyl.

In another aspect, the invention is directed to pharmaceutical compositions comprising one or more compounds of Formula VI:

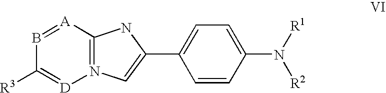

or a pharmaceutically acceptable salt thereof,

wherein:

A, B and D are CH or N,

provided that at least one, no more than two of A, B and D is N;

R³ is selected from the group consisting of —CH₃, hydrogen, halogen, halo(C₁₋₄)alkyl, halo(C₁₋₄)alkyl amino, halo(C₁₋₄)alkyl(C₁₋₄ alkyl)amino and Sn(alkyl)₃;

R¹ and R² are independently hydrogen, C₁₋₄ alkyl, C₂₋₄ aminoalkyl, C₁₋₄ haloalkyl, haloarylalkyl, or R¹ and R² are taken together with the nitrogen to which they are attached to form a 5- to 7-member heterocyclic ring optionally having O, S or NR⁵ in said ring, where R⁵ is hydrogen or C₁₋₄ alkyl.

The above description of compounds of Formula VI include the following proviso, wherein in each instance, the halogen is other than a radiolabeled halogen.

Useful values of R³ include those already listed above. More preferably, R³ is halogen, C₁₋₄ alkyl, hydrogen or Sn(alkyl)₃.

In a preferred group of compounds, A and B are CH, and D is N. In another preferred group of compounds, A and D are CH, and B is N. In another preferred group of compounds, B and D are CH, and A is N.

Preferred compounds are those of Formula VI wherein R¹ and R² are independently one of hydrogen, C₁₋₄ alkyl, C₁₋₄ haloalkyl, halophenyl(C₁₋₄)alkyl, or are taken together with the nitrogen to which they are attached to form a 5- to 7-member heterocyclic ring optionally having O or NR⁶ in said ring, where R⁶ is hydrogen or C₁₋₄ alkyl. Useful values of R¹ and R² include, independently, hydrogen, methyl, ethyl, propyl, butyl, t-butyl, isobutyl, 3-fluoropropyl, 4-fluorobutyl, or 4-fluorobenzyl, or R¹ and R² are taken together with the nitrogen to which they are attached to form a piperidinyl ring having NR⁶ in said ring, where R⁶ is hydrogen or methyl. Most preferably R¹ and R² are methyl.

In another aspect, the invention is directed to pharmaceutical compositions comprising one or more compounds of Formula VII: 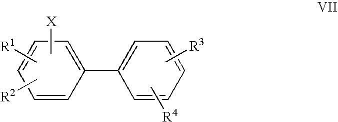
or a pharmaceutically acceptable salt thereof, wherein

R¹, R² and R³ are independently selected from the group consisting of hydrogen, halogen, C₁₋₅ alkyl, cyano, carboxy(C₁₋₅)alkyl, trifluoromethyl, nitro, methylamino, dimethylamino, halo(C₁₋₅)alkyl, hydroxy(C₁₋₅)alkyl, —(Bu)₃Sn—, (Bu)₃Sn(C₁₋₅)alkyl and formyl,
R⁴ is selected from the group consisting of:
a. C₁₋₅ alkylthio,
b. halo(C₁₋₅)alkyl,
c. halo(C₁₋₅)alkoxy,
d. carboxy(C₁₋₅)alkyl,
e. hydroxy,
f. C₁₋₅ alkoxy,
g. hydroxy(C₁₋₅)alkyl,
h. NR⁵R⁶, wherein
   R⁵ and R⁶ are independently hydrogen, fluoro(C₁₋₅)alkyl or C₁₋₅ alkyl,
i. phenyl(C₁₋₅)alkyl,
j. C₆₋₁₀ aryl,
k. heteroaryl,
l. heterocycle,
m. heterocycle(C₁₋₅)alkyl, and
n. C₃₋₆ cycloalkyl,
   wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,

and, X is selected from the group consisting of hydrogen, halogen, halo(C₁₋₄)alkyl, halo(C₁₋₄)alkyl amino, halo(C₁₋₄)alkyl(C₁₋₄ alkyl)amino and Sn(alkyl)₃.

The above description of compounds of Formula VII include the following provisos, wherein in each instance, the halogen is other than a radiolabeled halogen, and if R4 is other than NR5R6, then R1 is methylamino or dimethylamino.

Useful values of X include those already listed above. Preferably, X is hydrogen, halogen or Sn(alkyl)₃.

In a preferred embodiment, R3 is hydrogen.

With respect to the relative positions of any substituent on an aromatic ring, it is envisioned that R1, R2, R3, R4 and X may occur at ortho, meta, or para positions relative to the linkage bond between the aromatic rings. It is also envisioned that in preferred embodiments wherein each aromatic ring has one substituent, the ortho, meta or para position of each substituent is independent of the substituent on the opposite ring. In compounds containing one substituent on each ring it is preferred that each substituent is independently either in a meta or para position relative to said linkage bond. Most preferably, one substituent on each ring is in the para position.

In another aspect, the invention is directed to pharmaceutical compositions comprising one or more compounds of Formula VIII: 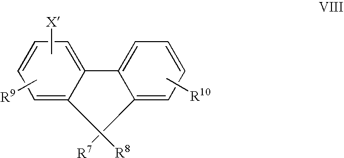
or a pharmaceutically acceptable salt thereof, wherein:
R⁹ and R¹⁰ are independently selected from the group consisting of:
a. hydrogen,
b. C₁₋₅ alkyl,
c. cyano,
d. trifluoromethyl,
e. nitro,
f. halogen,
g. hydroxy(C₁₋₅)alkyl,
h. halo(C₁₋₅)alkyl,
i. C₁₋₅ alkylthio,
j. halo(C₁₋₅)alkoxy,
k. carboxy(C₁₋₅)alkyl,
l. hydroxy,
m. C₁₋₅ alkoxy,
n. NR¹¹R¹², wherein
   R¹¹ and R¹² are independently hydrogen, fluoro(C₁₋₅)alkyl or C₁₋₅ alkyl,
o. phenyl(C₁₋₅)alkyl,
p. C₆₋₁₀ aryl,
q. heteroaryl,
r. heterocycle,
s. heterocycle(C₁₋₅)alkyl, and
t. C₃₋₆ cycloalkyl,
   wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,

R⁷ and R⁸ are independently selected from the group consisting of hydrogen, hydroxy, hydroxy(C₁₋₅)alkyl, C₁₋₅ alkyl, C₁₋₅ alkoxy, halogen, carboxy(C₁₋₅)alkyl, trifluoromethyl, and halo(C₁₋₅)alkyl, phenyl(C₁₋₅)alkyl, C₃₋₆ cycloalkyl, heterocycle(C₁₋₅)alkyl, or R⁷ and R⁸ can be taken together to form a carbonyl, and

X′ is selected from the group consisting of hydrogen, halogen, halo(C₁₋₄)alkyl, halo(C₁₋₄)alkyl amino, halo(C₁₋₄)alkyl(C₁₋₄ alkyl)amino and Sn(alkyl)₃.

The above description of compounds of Formula VIII include the following proviso, wherein in each instance, the halogen is other than a radiolabeled halogen, and at least one of R⁹ and R¹⁰ is NR¹¹R¹², wherein R¹¹ and R¹² are as described above.

Useful compounds falling within the scope of Formula VIII include compounds wherein R⁹ and R¹⁰ are independently selected from the group as described above. Preferably, R⁹ is hydrogen, halogen, hydroxy(C₁₋₅)alkyl, halo(C₁₋₅)alkyl or C₁₋₅ alkyl. Most preferably, R⁹ is hydrogen. Preferably, R¹⁰ is selected from the group consisting of cyano, nitro, and NR¹¹R¹², wherein R¹¹ and R¹² are independently hydrogen, halo(C₁₋₅)alkyl or C₁₋₅ alkyl. The most useful value of R¹⁰ is NR¹¹R¹², wherein R¹¹ and R¹² are independently hydrogen or C₁₋₅ alkyl. In this embodiment it is preferred that R¹¹ and R¹² are independently hydrogen, methyl or ethyl. Also preferred are compounds wherein R¹⁰ is NR¹¹R¹² wherein R¹¹ and R¹² are independently hydrogen, methyl or ethyl, X′ is hydrogen, and R⁹ is hydrogen.

Useful compounds are those of Formula VIII wherein R⁷ and R⁸ are independently selected from the group as described above. Preferably, R⁷ and R⁸ are independently hydrogen, hydroxyl, hydroxy(C₁₋₅)alkyl, halogen, halo(C₁₋₅)alkyl or C₁₋₅ alkyl, or R⁷ and R⁸ are taken together to form a carbonyl. More preferably, R⁷ and R⁸ are independently selected from the group consisting of hydrogen and hydroxyl or are taken together to form a carbonyl. In an especially preferred embodiment, R⁷ and R⁸ are both hydrogen.

Useful values of X′ include those already listed above. Preferably, X′ is hydrogen, halogen or Sn(alkyl)₃.

With respect to the relative positions of any substituent on an aromatic ring, it is envisioned that R⁹, R¹⁰ and X′ may occur at ortho, meta, or para positions relative to the linkage bond between the aromatic rings. It is also envisioned that in preferred embodiments wherein each aromatic ring has one substituent, the ortho, meta or para position of each substituent is independent of the substituent on the opposite ring. In compounds containing one substituent on each ring it is preferred that each substituent is independently either in a meta or para position relative to said linkage bond. Most preferably, one substituent on each ring is in the para position.

In another aspect, the invention is directed to pharmaceutical compositions comprising one or more compounds of Formula IX: 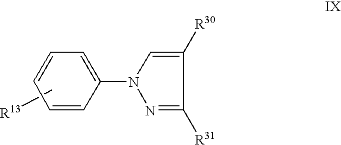
or a pharmaceutically acceptable salt thereof, wherein:
R¹³ is selected from the group consisting of:
a. C₁₋₅ alkyl,
b. cyano,
c. trifluoromethyl,
d. nitro,
e. halo(C₁₋₅)alkyl,
f. C₁₋₅ alkylthio,
g. hydroxy(C₁₋₅)alkyl,
h. halogen,
i. halo(C₁₋₅)alkoxy,
j. carboxy(C₁₋₅)alkyl,
k. hydroxy,
l. C₁₋₅ alkoxy,
m. NR¹⁴R¹⁵, wherein
   R¹⁴ and R¹⁵ are independently hydrogen, halo(C₁₋₅)alkyl or C₁₋₅ alkyl,
n. phenyl(C₁₋₅)alkyl,
o. C₆₋₁₀ aryl,
p. heteroaryl,
q. heterocycle,
r. heterocycle(C₁₋₅)alkyl, and
s. C₃₋₆ cycloalkyl,
   wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
      and,

R³⁰ and R³¹ are selected from the group consisting of hydrogen, hydroxy, hydroxy(C₁₋₅)alkyl, C₁₋₅ alkyl, C₁₋₅ alkoxy, (C₁₋₅)alkyl carboxy, halogen, carboxy(C₁₋₅)alkyl, trifluoromethyl, halo(C₁₋₅)alkyl, phenyl(C₁₋₅)alkyl, C₃₋₆ cycloalkyl and heterocycle(C₁₋₅)alkyl.

The above description of compounds of Formula IX include the following proviso, wherein in each instance, the halogen is other than a radiolabeled halogen.

Useful compounds of Formula IX are those compounds wherein R¹³ is described above. In preferred compounds, R¹³ is NR¹⁴R¹⁵, wherein R¹⁴ and R¹⁵ are independently selected from the group consisting of hydrogen, halo(C₁₋₅)alkyl or C₁₋₅ alkyl. More preferably, wherein R¹⁴ and R¹⁵ are independently selected from the group consisting of hydrogen, methyl and fluoro(C₁₋₅)alkyl.

With respect to the relative positions of any substituent on an aromatic ring, it is envisioned that R¹³ can occur at any available position. The position of R¹³ is independent of the position of any substituent on the opposite ring. In preferred compounds R¹³ is either in a meta or para position relative to the linkage bond between the two aromatic rings. Most preferably, R¹³ is in the para position.

Useful values of R³⁰ are as described above. Preferred values include a non-radiolabeled halogen, C₁₋₅ alkyl, and halo(C₁₋₅)alkyl.

Useful values of R³¹ are as described above. Preferably, R³¹ is C₁₋₅ alkyl. Most preferably, R³¹ is methyl.

Useful compounds falling within the scope of Formula IX include compounds wherein R¹, R² and R³ are independently selected from the group as described above. Preferably, R¹, R² and R³ are hydrogen, C₁₋₅ alkyl, halo(C₁₋₅)alkyl, (Bu)₃Sn— or (Bu)₃Sn(C₁₋₅)alkyl.

Useful compounds of Formula IX also include those compounds wherein R⁴ is as described above. Preferable values of R⁴ under the scope of Formula I include halo(C₁₋₅)alkyl, hydroxy, hydroxy(C₁₋₅)alkyl, C₁₋₅ alkoxy, and NR⁵R⁶, wherein R⁵ and R⁶ are independently hydrogen, halo(C₁₋₅)alkyl or C₁₋₅ alkyl. More preferably, R⁴ is NR⁵R⁶, wherein R⁵ and R⁶ are independently hydrogen, halo(C₁₋₅)alkyl or C₁₋₅ alkyl. In a most preferable embodiment, R¹, R² and R³ are hydrogen or the tetradentate metal ligand moiety described above, and R⁴ is NR⁵R⁶, wherein R⁵ and R⁶ are independently hydrogen, halo(C₁₋₅)alkyl or C₁₋₁₅ alkyl.

Other preferred compounds include those compounds where R¹ is methylamino or dimethylamino, R² is hydrogen, R³ is halo(C₁₋₅)alkyl or (Bu₃)Sn(C₁₋₅)alkyl, R⁴ is hydroxy or hydroxy(C₁₋₅)alkyl, and X is hydrogen. In these embodiments, it is more preferred that R¹ and R⁴ are in the para position relative to the bridge, and R³ is in the ortho position relative to R⁴. In more preferred embodiments, R¹ is dimethylamino. More preferred embodiments also include those compounds wherein R³ is fluoro(C₁₋₅)alkyl. Most preferably, R³ is fluoromethyl or fluoroethyl. In preferred embodiments, R⁴ is hydroxy, methoxy or ethoxy. Most preferably, R⁴ is hydroxy.

In another aspect, the invention is directed to pharmaceutical compositions comprising one or more compounds of Formula X: 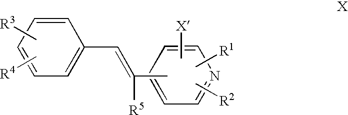
or a pharmaceutically acceptable salt thereof, wherein: R⁵ is hydrogen or C₁₋₄ alkyl; R¹, R² and R³, in each instance, is independently selected from the group consisting of hydrogen, hydroxy, halogen, C₁₋₄ alkyl, C₁₋₄ alkoxy, cyano, carboxy(C₁₋₅)alkyl, trifluoromethyl, nitro, methylamino, dimethylamino, halo(C₁₋₄)alkyl, and formyl;
R⁴ is selected from the group consisting of:
a. C₁₋₄ alkylthio,
b. C₁₋₄ alkylsulfonyl,
c. hydroxy,
d. C₁₋₄ alkoxy,
e. NR⁶R⁷, wherein
   R⁶ and R⁷ are independently hydrogen or C₁₋₄ alkyl,
f. phenyl(C₁₋₄)alkyl,
g. C₆₋₁₀ aryl,
h. heteroaryl,
i. heterocycle,
j. heterocycle(C₁₋₄)alkyl, and
k. C₃₋₆ cycloalkyl,
   wherein said phenyl(C₁₋₄)alkyl, C₆₋₁₀ aryl, heteroaryl, heterocycle, heterocycle(C₁₋₄)alkyl or C₃₋₆ cycloalkyl is substituted with one of the following: C₁₋₄ alkylthio, C₁₋₄ alkyl sulfonyl, methoxy, hydroxy, dimethylamino or methylamino;
or R³ and R⁴ are taken together to form an optionally substituted aryl or heteroaryl ring, wherein said ring is attached at adjacent carbons on the core pyridine ring;
   provided that, in each instance, said halogen is other than a radiolabeled halogen; and if R⁴ is other than NR⁶R⁷, then R³ is methylamino or dimethyl amino.

Preferred values are also those values described above under Formula I for each of R¹, R², R³, R⁴ and R⁵. In another preferred embodiment, R¹, R² and R³ are each independently selected from the group consisting of hydrogen, and C₁₋₄ alkyl. In this embodiment, R⁵ is preferably methyl or hydrogen, and R³ is hydrogen. In this embodiment, R⁴ is preferably NR⁶R⁷, wherein R⁶ and R⁷ are independently hydrogen or C₁₋₄ alkyl. Most preferably, R¹, R² and R³ are each hydrogen and R⁶ and R⁷ are each methyl.

Preferred compounds of Formulae I, II, m, IV, V, VI, VII, VIII, IX and X include:

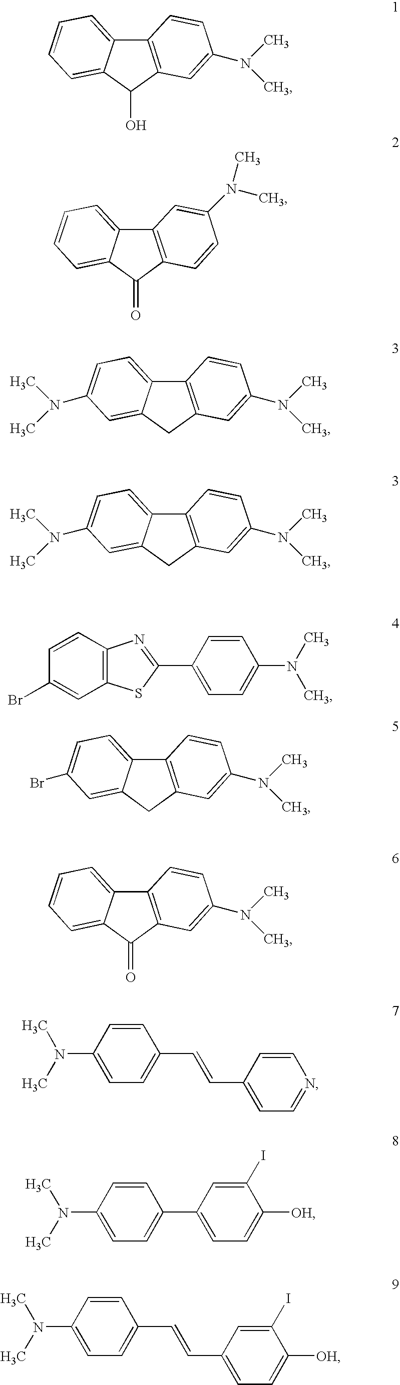

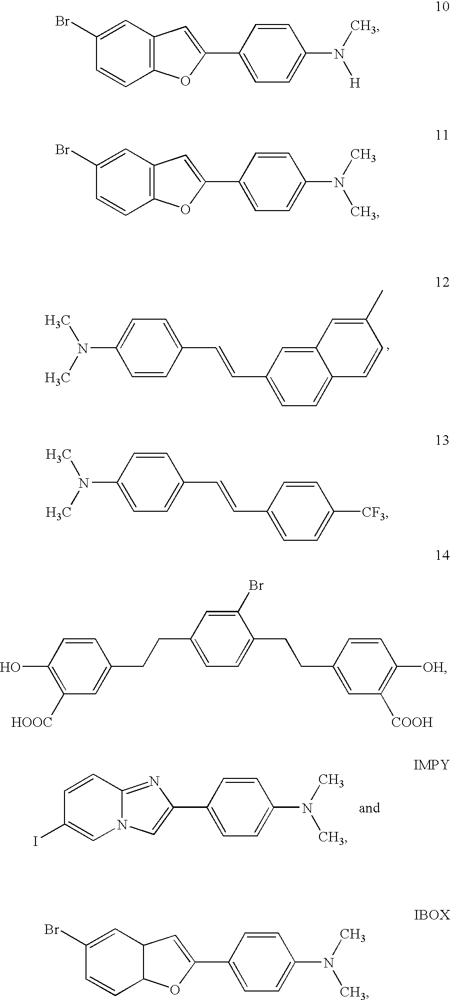

In another aspect, the present invention provides a method of rescuing cells from amyloid associated toxicity. This method provides for inhibiting the aggregation of amyloid proteins to form amyloid deposits by administering to a patient an amyloid inhibiting amount of a compound of the above Formula I, I, III, IV, V, VI, VII, VIII, IX or X.

In the first step of the present method of inhibiting amyloid aggregation, one or more compounds of Formula I, H, III, IV, V, VI, VII, VIII, IX or X is introduced into a tissue or a patient. The compound is typically part of a pharmaceutical composition and is administered to the tissue or the patient by methods well known to those skilled in the art.

For example, the compound can be administered either orally, rectally, parenterally (intravenous, by intramuscularly or subcutaneously), intracisternally, intravaginally, intraperitoneally, intravesically, locally (powders, ointments or drops), or as a buccal or nasal spray.

The administration of the compound to a patient can be by a general or local administration route. For example, the compound may be administered to the patient such that it is delivered throughout the body. Alternatively, the compound can be administered to a specific organ or tissue of interest. The term “tissue” means a part of a patient's body. Examples of tissues include the brain, heart, liver, blood vessels, and arteries.

The term “patient” means humans and other animals.

Those skilled in the art are readily able to determine an amyloid inhibiting amount by simply administering a compound of Formula I, II, III, IV, V, VI, VII, VIII, IX or X to a patient in increasing amounts until the growth of amyloid deposits is decreased or stopped. The rate of growth can be assessed using well-known techniques such as imaging or by taking a tissue sample from a patient and observing the amyloid deposits therein. The compounds of the present invention can be administered to a patient at dosage levels in the range of about 0.1 to about 1,000 mg per day. For a normal human adult having a body weight of about 70 kg, a dosage in the range of about 0.01 to about 100 mg per kilogram of body weight per day is sufficient. The specific dosage used, however, can vary. For example, the dosage can depend on a number of factors including the requirements of the patient, the severity of the condition being treated, and the pharmacological activity of the compound being used. The determination of optimum dosages for a particular patient is well known to those skilled in the art.

It is also to be understood that the present invention is considered to include the use and administration of stereoisomers as well as optical isomers, e.g. mixtures of enantiomers as well as individual enantiomers and diastereomers, which arise as a consequence of structural asymmetry in selected compounds of the present series.

Prior to administration, the compounds of Formula I, II, III, IV, V, VI, VII, VIII, IX or X may also be solvated, especially hydrated. Hydration may occur during manufacturing of the compounds or compositions comprising the compounds, or the hydration may occur over time due to the hygroscopic nature of the compounds. In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

When any variable occurs more than one time in any constituent or in Formula I, II, III, IV, V, VI, VII, VIII, IX or X its definition on each occurrence is independent of its definition at every other occurrence. Also combinations of substituents and/or variables are permissible only if such combinations result in stable compounds.

The term “alkyl” as employed herein by itself or as part of another group refers to both straight and branched chain radicals of up to 8 carbons, preferably 6 carbons, more preferably 4 carbons, such as methyl, ethyl, propyl, isopropyl, butyl, t-butyl, and isobutyl.

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 6 carbon atoms in length, more preferably 1-4 carbon atoms in length.

The term “monoalkylamine” as employed herein by itself or as part of another group refers to an amino group which is substituted with one alkyl group as defined above.

The term “dialkylamine” as employed herein by itself or as part of another group refers to an amino group which is substituted with two alkyl groups as defined above.

The term “halo” or “halogen” employed herein by itself or as part of another group refers to non-radiolabeled chlorine, bromine, fluorine or iodine.

The term “haloalkyl” as employed herein refers to any of the above alkyl groups substituted by one or more chlorine, bromine, fluorine or iodine with fluorine and chlorine being preferred, such as chloromethyl, iodomethyl, trifluoromethyl, 2,2,2-trifluoroethyl, and 2-chloroethyl.

The term “alkylthio” as employed herein by itself or as part of another group refers to a thioether of the structure: R—S, wherein R is a C₁₋₄ alkyl as defined above.

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 6 carbon atoms in length, more preferably 1-4 carbon atoms in length.

The term “alkylsulfonyl” as employed herein by itself or as part of another group refers to a sulfone of the structure: R—SO₂, wherein R is a C₁₋₄ alkyl as defined above.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl.

The term “heterocycle” or “heterocyclic ring”, as used herein except where noted, represents a stable 5- to 7-membered mono-heterocyclic ring system which may be saturated or unsaturated, and which consists of carbon atoms and from one to three heteroatoms selected from the group consisting of N, O, and S, and wherein the nitrogen and sulfur heteroatom may optionally be oxidized. Especially useful are rings contain one nitrogen combined with one oxygen or sulfur, or two nitrogen heteroatoms. Examples of such heterocyclic groups include piperidinyl, pyrrolyl, pyrrolidinyl, imidazolyl, imidazinyl, imidazolidinyl, pyridyl, pyrazinyl, pyrimidinyl, oxazolyl, oxazolidinyl, isoxazolyl, isoxazolidinyl, thiazolyl, thiazolidinyl, isothiazolyl, homopiperidinyl, homopiperazinyl, pyridazinyl, pyrazolyl, and pyrazolidinyl, most preferably thiamorpholinyl, piperazinyl, and morpholinyl.

The term “heteroatom” is used herein to mean an oxygen atom (“O”), a sulfur atom (“S”) or a nitrogen atom (“N”). It will be recognized that when the heteroatom is nitrogen, it may form an NR^{a}R^{b} moiety, wherein R^{a} and R^{b} are, independently from one another, hydrogen or C₁₋₄ alkyl, C₂₋₄ aminoalkyl, C₁₋₄ halo alkyl, halo benzyl, or R¹ and R² are taken together to form a 5- to 7-member heterocyclic ring optionally having O, S or NR^{c} in said ring, where R^{c} is hydrogen or C₁₋₄ alkyl.

The term “heteroaryl” as employed herein refers to groups having 5 to 14 ring atoms; 6, 10 or 14 B electrons shared in a cyclic array; and containing carbon atoms and 1, 2 or 3 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, 3-carbolinyl, phenanthridinyl, acridinyl, perimidinyl, phenanthrolinyl, phenazinyl, isothiazolyl, phenothiazinyl, isoxazolyl, furazanyl and phenoxazinyl groups).

The term “aralkyl” or “arylalkyl” as employed herein by itself or as part of another group refers to C₁₋₆alkyl groups as discussed above having an aryl substituent, such as benzyl, phenylethyl or 2-naphthylmethyl.

The term “hydroxy(C₁₋₅)alkyl” as employed herein refers to an alkyl chain connected to a compound of one of the general formulas disclosed herein via the ring or a chain of the compound, wherein the distal portion of the alkyl chain of the group contains a hydroxy moiety. The alkyl chain can contain any number of carbons, but preferably the number of carbons in the alkyl chain is from 1 to 5.

The term “haloalkyl” as employed herein refers to any of the above alkyl groups substituted by one or more chlorine, bromine, fluorine or iodine with fluorine and chlorine being preferred, such as chloromethyl, iodomethyl, trifluoromethyl, 2,2,2-trifluoroethyl, and 2-chloroethyl.

The term “pharmaceutically acceptable salt” as used herein refers to those carboxylate salts or acid addition salts of the compounds of the present invention which are, within the scope of sound medical judgement, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively nontoxic, inorganic and organic acid addition salts of compounds of the present invention. Also included are those salts derived from non-toxic organic acids such as aliphatic mono and dicarboxylic acids, for example acetic acid, phenyl-substituted alkanoic acids, hydroxy alkanoic and alkanedioic acids, aromatic acids, and aliphatic and aromatic sulfonic acids. These salts can be prepared in situ during the final isolation and purification of the compounds or by separately reacting the purified compound in its free base form with a suitable organic or inorganic acid and isolating the salt thus formed. Further representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactiobionate and laurylsulphonate salts, propionate, pivalate, cyclamate, isethionate, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, Berge S. M., et al., Pharmaceutical Salts, J. Pharm. Sci. 66:1-19 (1977) which is incorporated herein by reference.)

Schemes 1-5 depict a synthetic route for forming compounds of Formula I using a Wittig reagent.

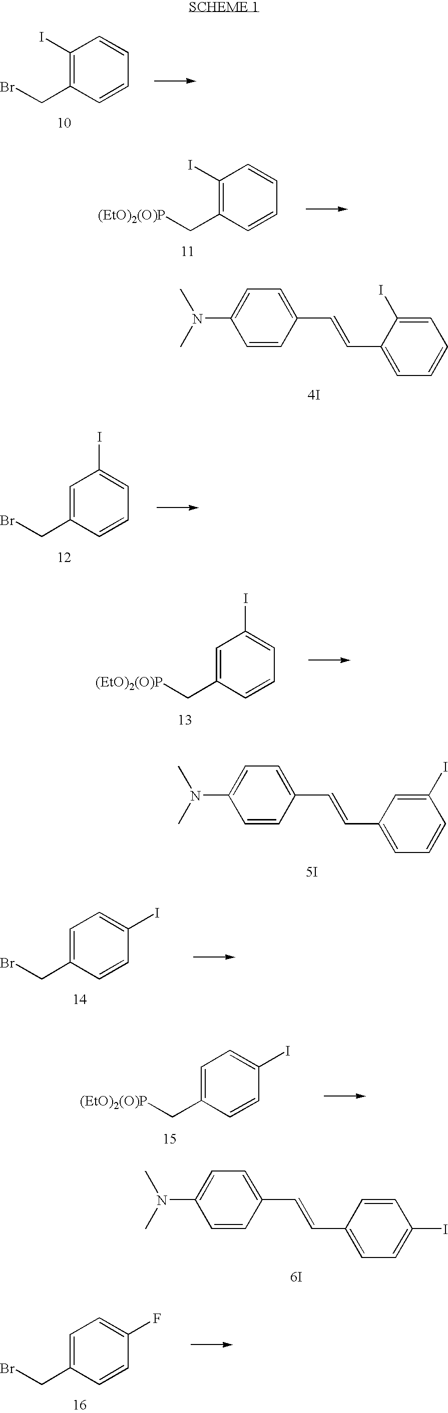

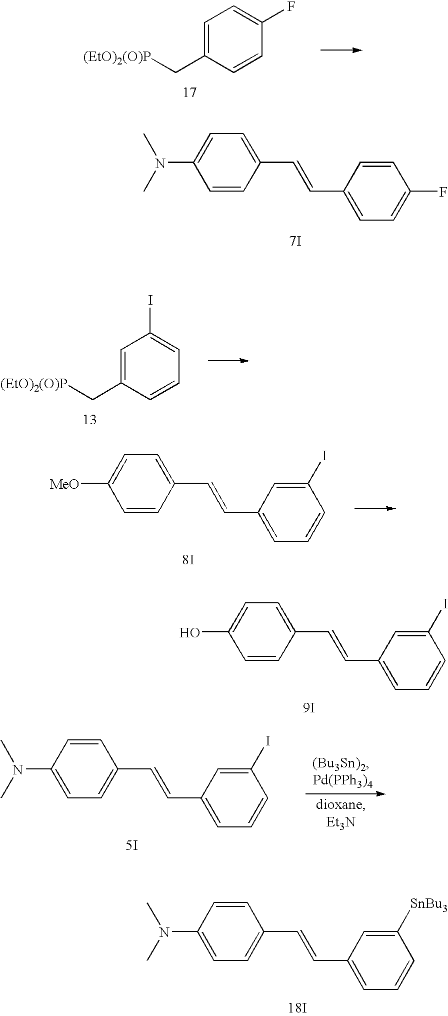

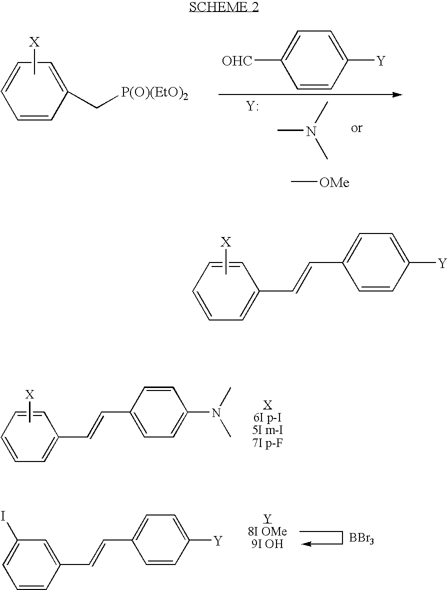

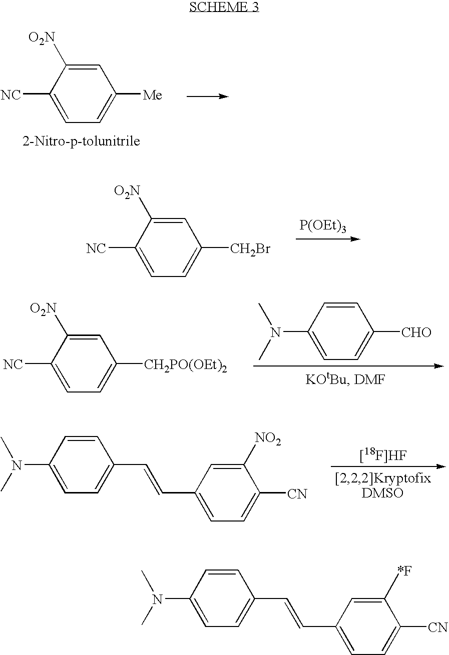

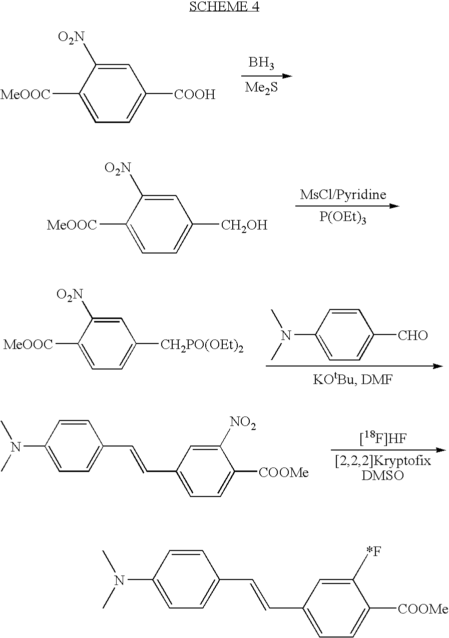

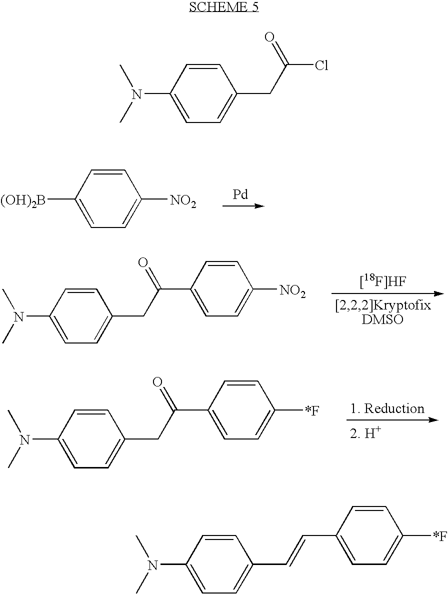

Scheme 6 depicts a synthetic route for forming derivatives of Formula II.

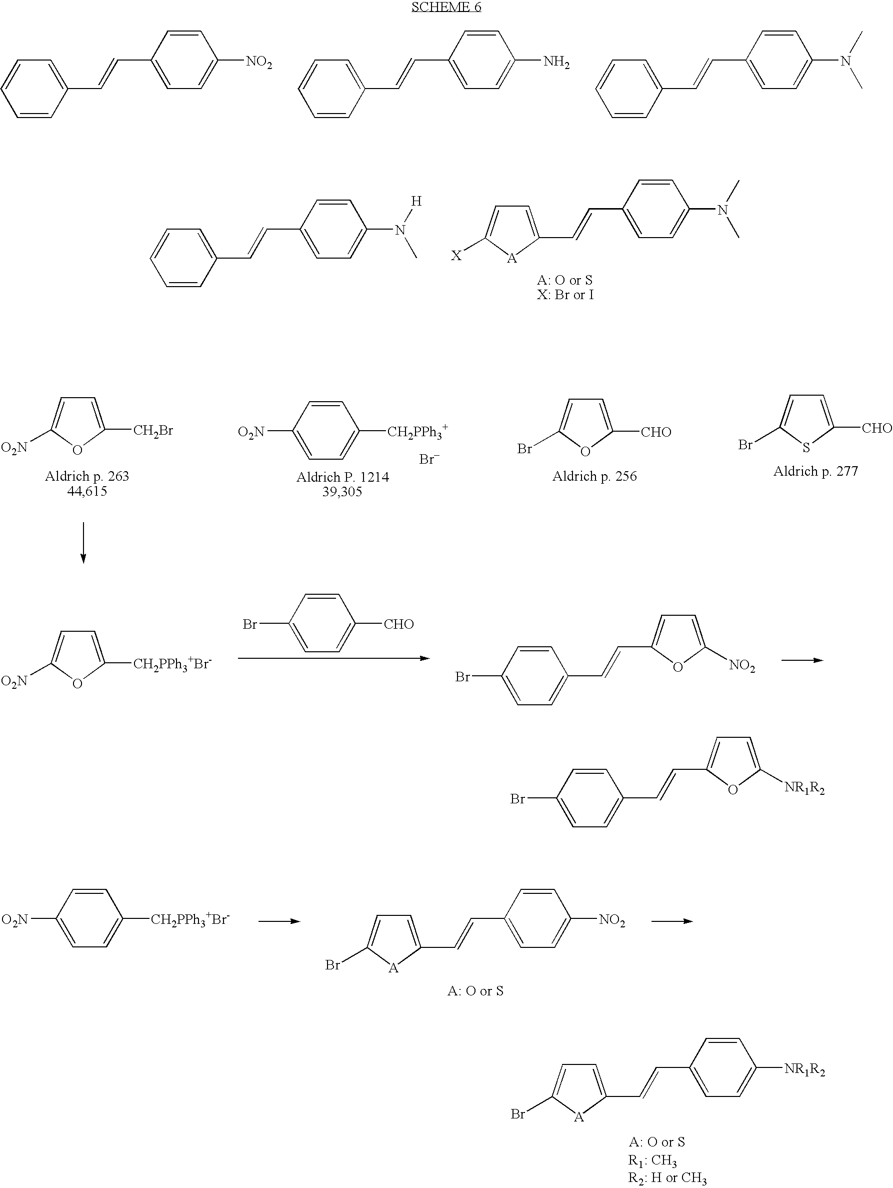

Compounds of Formula III, IV, V or VI can be prepared by following the methods. A first method is characterized by forming a benzothiazole of Formula III wherein Y is S by reacting a 2-aminothiophenol with either: a) a 4-aminobenzaldehyde in DMSO at a temperature in the range of 100° C.-220° C., and collecting said benzothiazole; or b) a 4-halobenzoic acid derivative in a solvent in the presence of polyphosphoric acid, collecting the product of this reaction, followed by reacting said product with an amine to form said benzothiazole, and collecting said benzothiazole; and optionally reacting a benzothiazole of Formula III wherein Y is S with (alkyl)₃Sn in a solvent in the presence of palladiumIIoxide to form a trialkylstannyl benzothiazole, and collecting the product of this reaction; and optionally reacting a trialkylstannyl benzothiazole of Formula III wherein Y is S with either: a) iodine in a solvent at ambient temperature, and extracting the product; or b) NaI in the presence of hydrogen peroxide, and extracting the product.

A second method is characterized by forming a benzoxazole of Formula III wherein Y is O by reacting a 2-amino-5-nitrophenol with a 4-aminobenzoic acid to form a nitro-substituted benzoxazole intermediate, and collecting said intermediate; followed by catalytic hydrogenation of said nitro group to an amino group, and collecting the product of this reaction; and reacting said product with NaNO₂ in the presence of H⁺ and potassium halide to produce a benzoxazole of Formula III wherein Y is O; and optionally reacting a benzoxazole of Formula III wherein Y is O with (alkyl)₃Sn in a solvent in the presence of palladiumIIoxide to form a trialkylstannyl benzoxazole, and collecting the product of this reaction; and optionally reacting a trialkylstannyl benzoxazole of Formula III wherein Y is O with either: a) iodine in a solvent at ambient temperature, and extracting the product; or b) NaI in the presence of hydrogen peroxide, and extracting the product.

A third method is characterized by forming a benzimidazole of Formula III wherein Y is N by reacting a 4-bromo-1,2-diaminobenzene with either: a) a 4-aminobenzaldehyde to form a benzimidazole of Formula III wherein Y is N, and collecting the product, or b) a 4-halobenzaldehyde to form an intermediate benzimidazole, and reacting said intermediate with a monoalkylamine, dialkylamine, or heterocyclic amine in the presence of palladiumIIoxide to form a benzimidazole of Formula III wherein Y is N, and collecting the product; and optionally reacting a benzimidazole of Formula I wherein Y is N with (alkyl)₃Sn in a solvent in the presence of palladiumIIoxide to form a trialkylstannyl benzimidazole, and collecting the product of this reaction; and optionally reacting a trialkylstannyl benzimidazole of Formula III wherein Y is N with either: a) iodine in a solvent at ambient temperature, and extracting the product; or b) NaI in the presence of hydrogen peroxide, and extracting the product.

A fifth method is characterized by forming an isoxazole of Formula IV wherein Y is O by reacting a 3-halo-2-hydroxy benzaldehyde with a substituted benzamine such as 4-(halomethyl)-benzamine to form a phenoxy benzyl ether intermediate, and collecting the intermediate; followed by reacting said intermediate in a solvent in the presence of NaOMe or NaOEt to form an isoxazole of Formula IV wherein Y is O, and collecting the product; and optionally reacting an isoxazole of Formula IV wherein Y is O with (alkyl)₃Sn in a solvent in the presence of palladiumIIoxide to form a trialkylstannyl isoxazole of Formula IV wherein Y is O, and collecting the product of this reaction; and optionally reacting a trialkylstannyl isoxazole of Formula IV wherein Y is O with either: a) iodine in a solvent at ambient temperature, and extracting the product; or b) NaI in the presence of hydrogen peroxide, and extracting the product.

A sixth method is characterized by forming an indole of Formula IV wherein Y is NR⁴ by reacting a 2-nitro-4-bromo toluene with N-isopropyl-2,2′-iminodiethanol to form a N,N-dimethyl-styryl-2-nitro-4-bromo benzene intermediate, followed by reacting said intermediate with an acid chloride in the presence of triethylamine to produce an α,β-unsaturated ketone, which undergoes intramolecular annulation by heating in dioxane/water, followed by reacting with sodium hydrosulfite to form an indole of Formula IV wherein Y is NR⁴, and collecting the product; and optionally reacting said indole with methyl iodide in the presence of sodium hydride to produce an indole of Formula IV wherein Y is NR⁴ where R⁴ is methyl, and collecting the product; and optionally reacting an indole of Formula IV wherein Y is NR⁴ with (alkyl)₃Sn in a solvent in the presence of palladiumIIoxide to form a trialkylstannyl indole of Formula IV wherein Y is NR⁴, and collecting the product of this reaction; and optionally reacting a trialkylstannyl indole of Formula IV wherein Y is NR⁴ with either: a) iodine in a solvent at ambient temperature, and extracting the product; or b) NaI in the presence of hydrogen peroxide, and extracting the product.

A seventh method characterized by forming an imidazo[1,2a]pyridine of Formula V by reacting 2-amino-5-bromo-pyridine with either: a) a 4′-halo-1-halo-benzophenone in a solvent in the presence of sodium bicarbonate to form an intermediate imidazo[1,2a]pyridine, and collecting the product of the reaction; followed by reacting said intermediate with a monoalkylamine, dialkylamine or heterocyclic amine in the presence of palladiumIIoxide to form an imidazo[1,2a]pyridine of Formula V, or b) a 4′-amino-1-halo-acetophenone in a solvent in the presence of sodium bicarbonate to form an imidazo[1,2a]pyridine of Formula V, and collecting the product of the reaction; and optionally reacting an imidazo[1,2a]pyridine of Formula V with (alkyl)₃Sn in a solvent in the presence of palladiumIIoxide to form a trialkylstannyl imidazo[1,2a]pyridine of Formula V, and collecting the product of this reaction; and optionally reacting a trialkylstannyl imidazo[1,2a]pyridine of Formula V with either: a) iodine in a solvent at ambient temperature, and extracting the product; or b) NaI in the presence of hydrogen peroxide, and extracting the product.

Schemes 7 and 8 depict a synthetic route for forming benzothiazoles of Formula III. Heating 5-bromo-2-amino-benzenethiol (Mital, R. L. and Jain, S. K., J Chem Soc (C):2148 (1969); Lin, A.-J. and Kasina, S., J Heterocycl Chem 18:759 (1981)) and 4-dimethylaminobenzaldehyde or 4-(4-methylpiperazin-1-yl)benzaldehyde (Tanaka, A., et al., J. Med. Chem. 41:2390 (1998)) in DMSO produced benzothiazoles, 1-III and 4-III. Using the same Pd(0)-catalyzed Br to tributyltin exchange reaction, these two bromo derivatives were successfully converted to the corresponding tributyltin derivatives 2-III and 5-III. They were successfully used in an iododestannylation reaction to produce the corresponding iodinated compounds 3-III and 6-III (yields were between 25-35%; the reactions were not optimized). Thus, the tributyltin derivatives served the useful purpose of converting bromo to iodo derivatives.

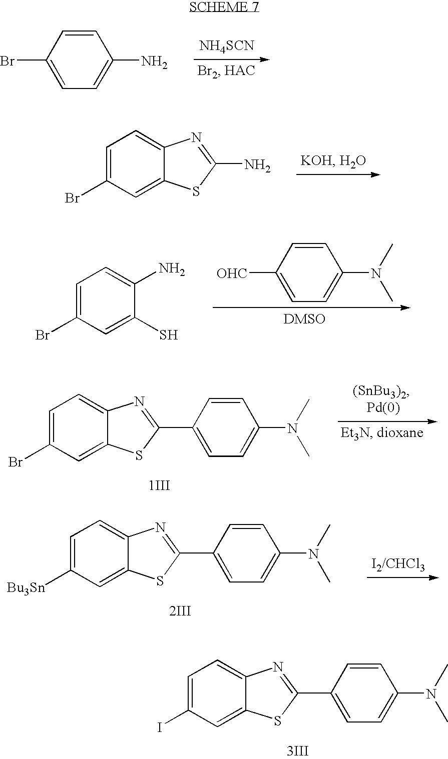

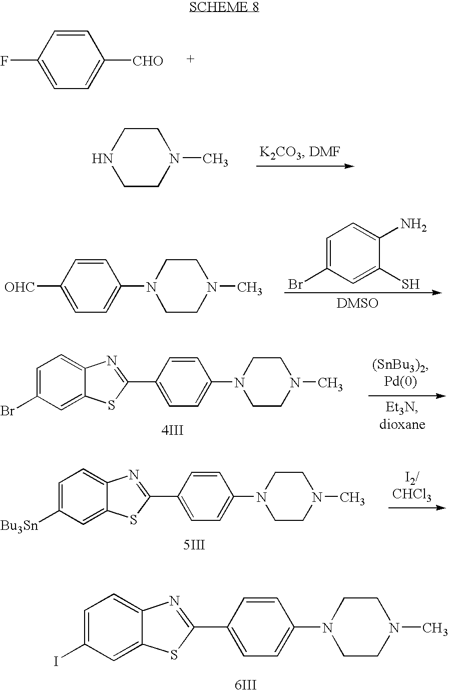

Scheme 9 depicts a synthetic route in which N-monomethylated amines are prepared, and thereafter employed in the parallel synthesis of disubstituted aminophenyl benzothiazole derivatives.

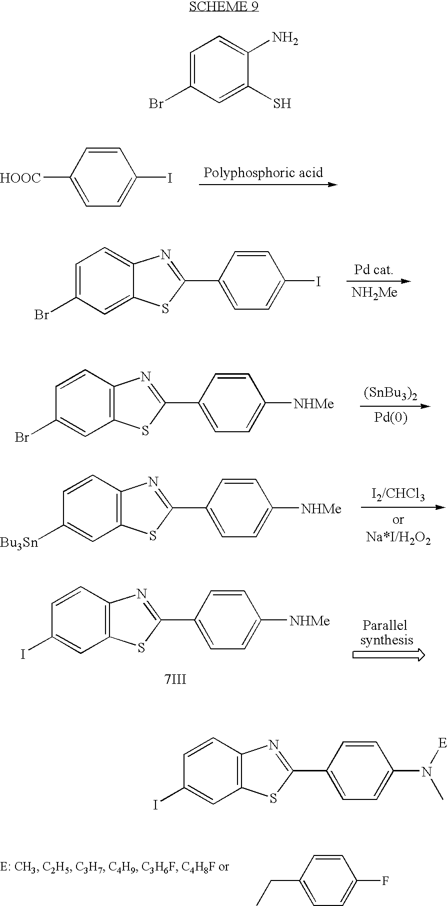

Schemes 10 through 12 depict synthetic routes for forming benzoxazoles of the present invention.

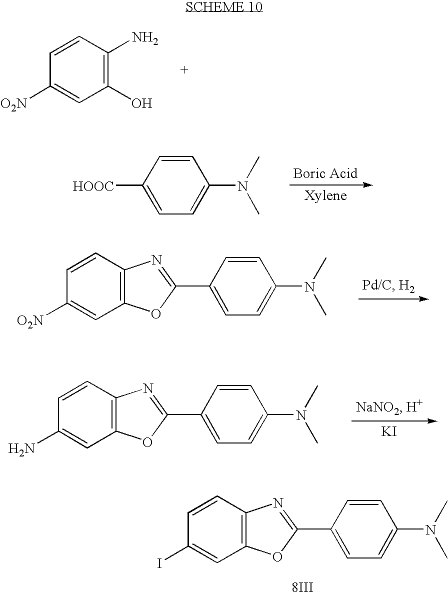

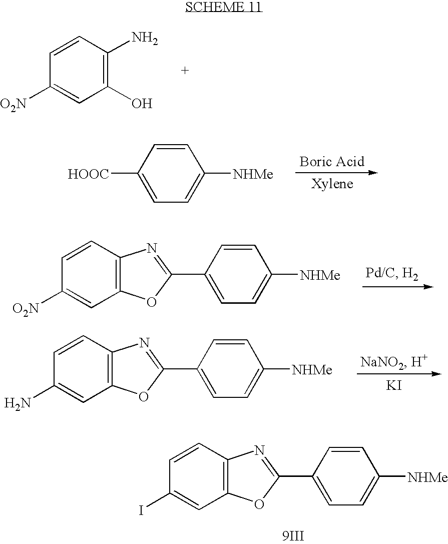

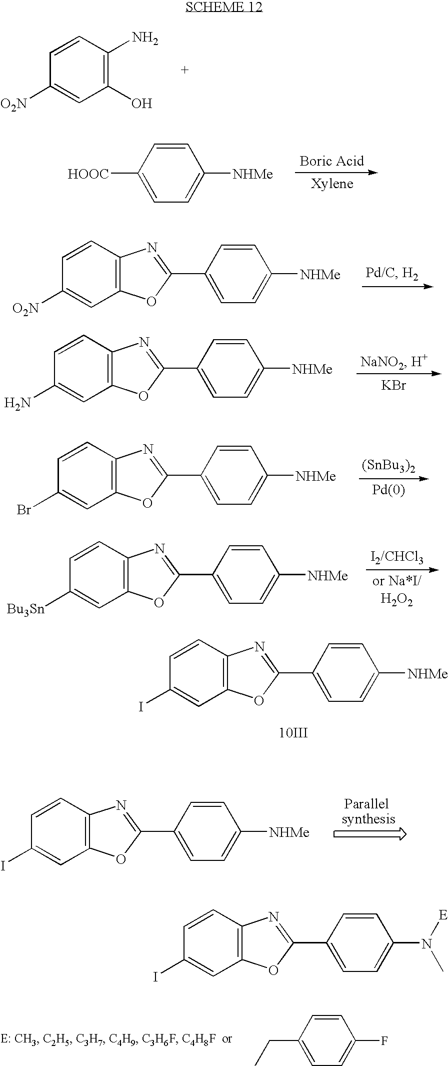

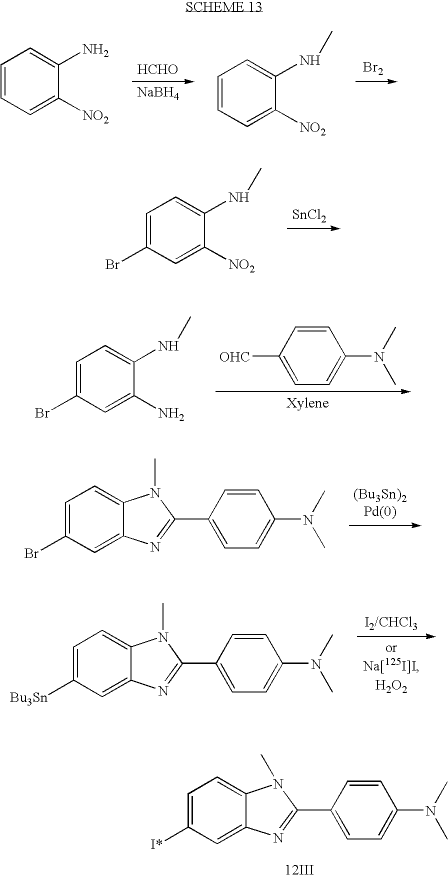

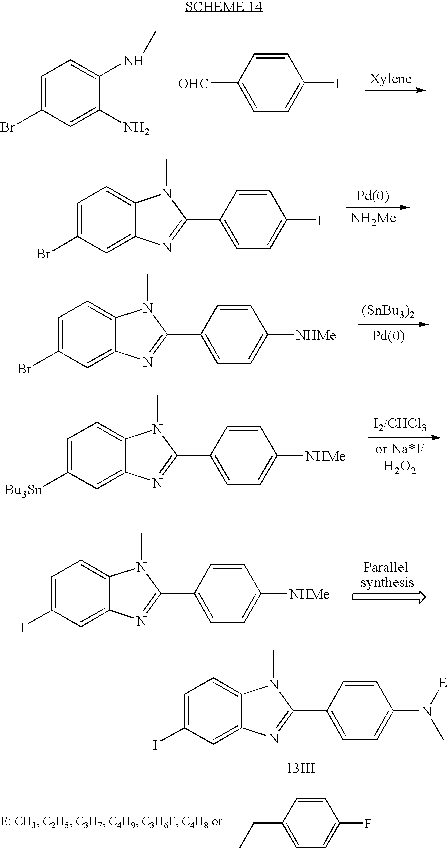

Schemes 15, 16, 17 and 18 depict synthetic routes for preparing Formula IV indole and benzofuran derivatives of the present invention.

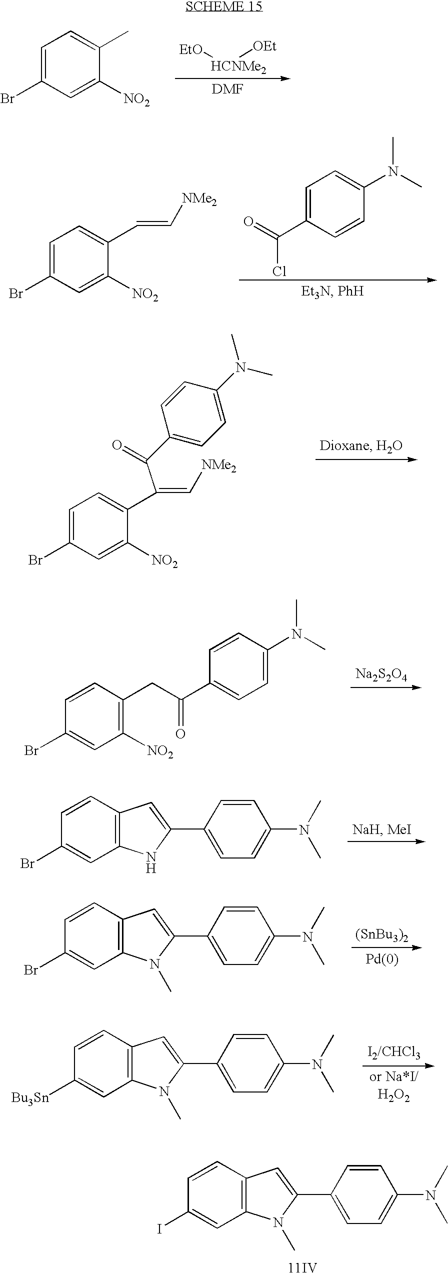

Scheme 16 depicts a synthetic route for forming benzofuran derivatives of the present invention. Alternatively, benzofurans can be prepared via an intramolecular Wittig Route (Twyman, et al., Tetrahedron Lett 40:9383 (1999)) as set forth in Scheme 17.

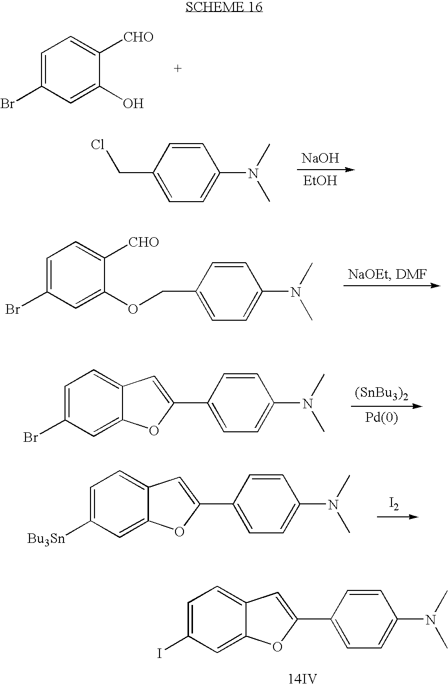

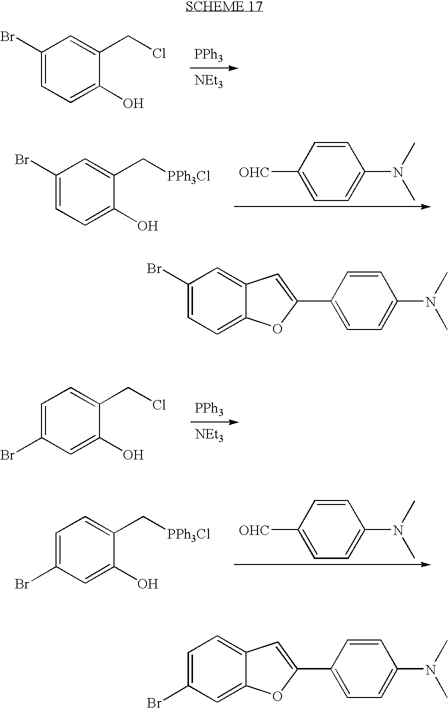

Scheme 18 provides a synthetic route for parallel synthesis of benzofuran derivatives of the present invention.

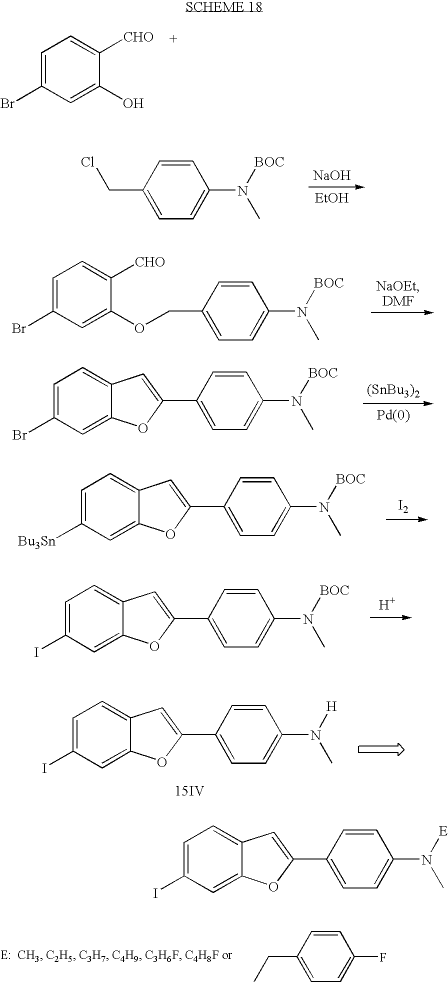

Schemes 19, 20, 21 and 22 are directed to imidazo[1,2,a]pyridine derivatives of Formula V.

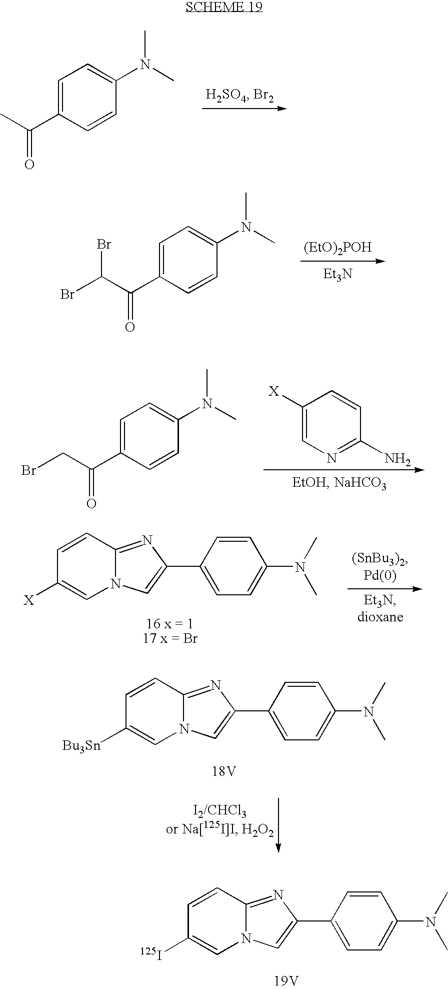

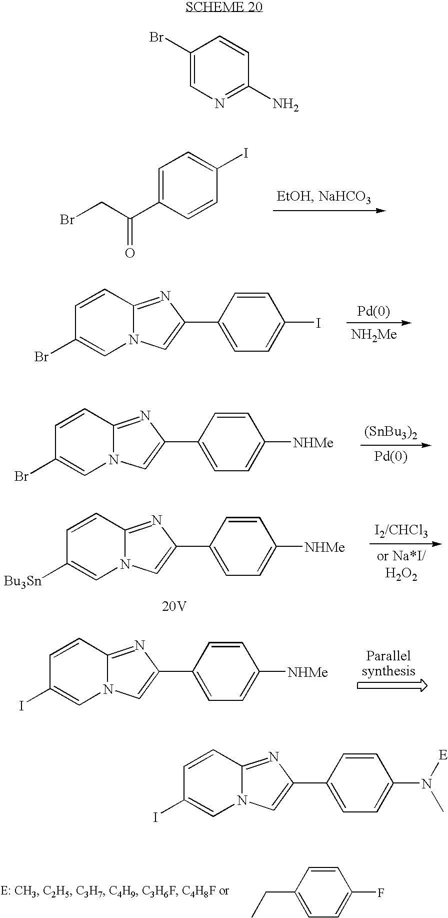

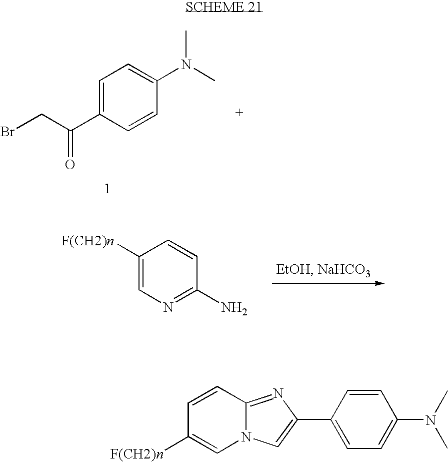

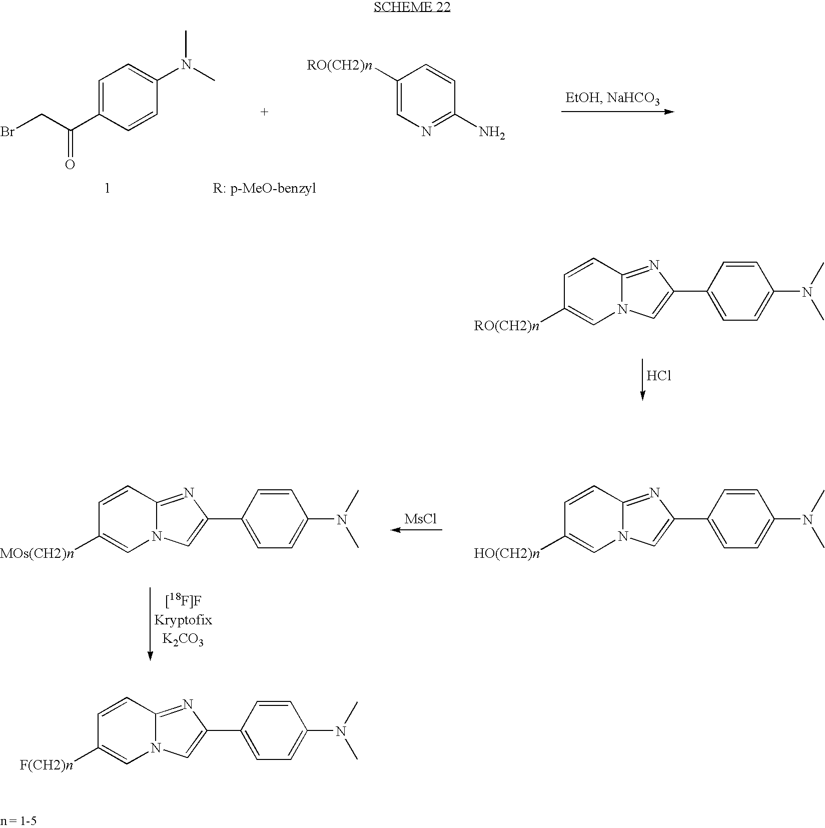

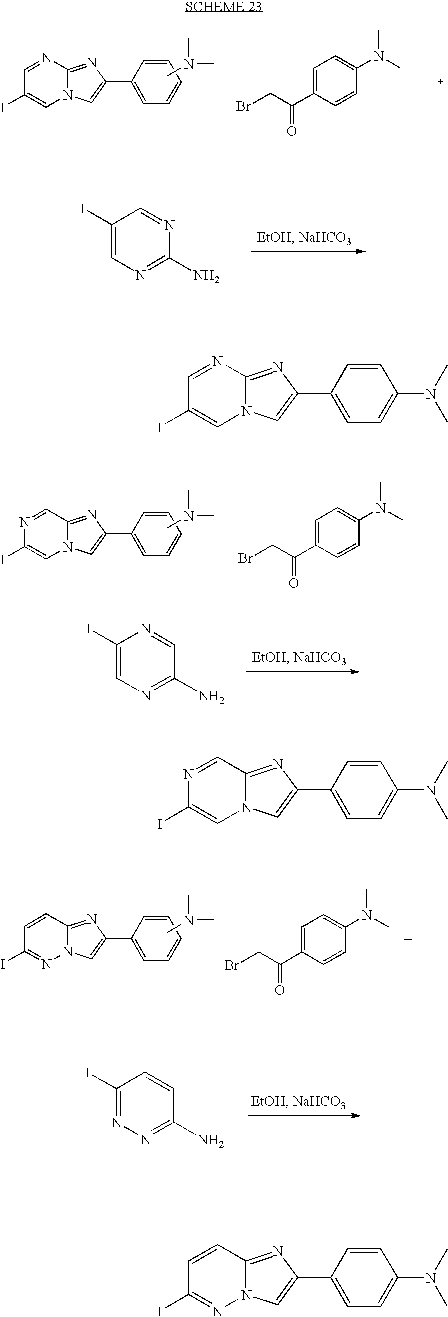

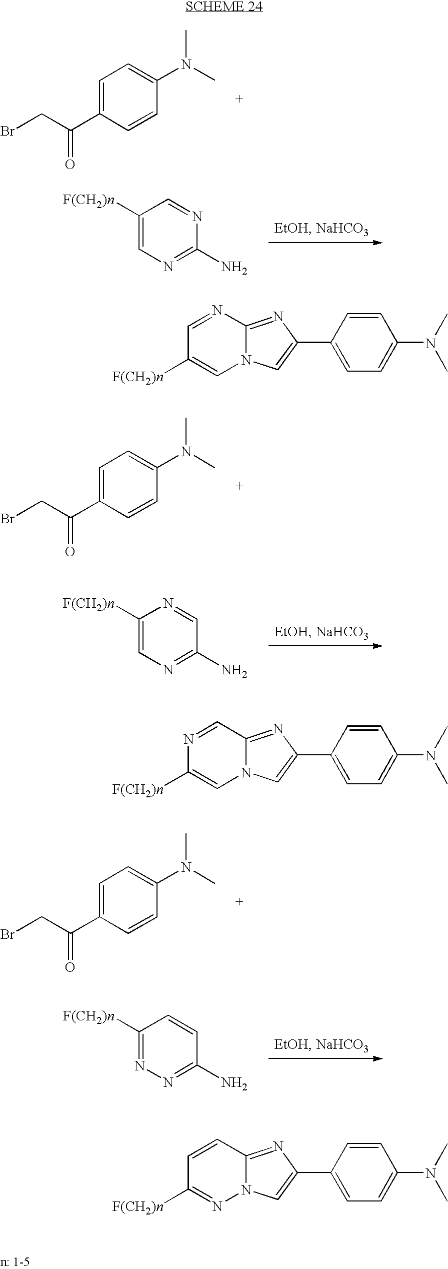

Formula VII and VIII compounds can be prepared by reactions described in Schemes 25, 26, 27, 28, 29 and 30. Synthesis of N,N-dimethylamino derivatives of fluorene was successfully achieved by a reductive methylation reaction shown in Scheme 25. Starting with 2- or 3-aminofluorenes, 1a-VIII through 1f-VIII, the amino group was converted to the N,N-dimethylamino group (2a-VIII-2f-VIII) in excellent yield (>90%) using paraformaldehyde in the presence of sodium cyanoborohydride as a reducing agent. A same-reaction was applied in the methylation of 9-fluorenones (Scheme 26) by which the amino-9-fluorenones were converted to N,N-dimethylamino-9-hydroxyfluorenes (3a-VIII-3d-VIII) in good yield (>80%). Under the reductive methylation condition, the keto group of fluorenone was reduced to 9-hydroxy group. To preserve the keto group of the 9-fluorenone, an alternative method was employed for the methylation reaction. Using methyliodide/K₂CO₃ in refluxing acetonitrile, the amino group of 9-fluorenone was methylated to the N,N-dimethylamino-9-fluorenones (4a-VIII-4-d-VIII) (Scheme 27). The yields for this methylation reaction were less predictable (ranging from 18-70%). To prepare the tributyltin, 5-VIII, the bromo derivative, 2d-VIII, was treated with bis(tributyltin) and Pd(Ph₃P)₂ in a mixed solvent of dioxane:triethylamine at 90° C. to give the desired tributyltin derivative, 5-VIII. Preparation of radioiodinated [¹²⁵I]2f-VIII was carried out by an iododestannylation reaction of 5, which was catalyzed by hydrogen peroxide (Scheme 28).

Scheme 25

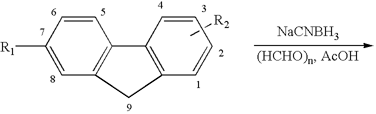

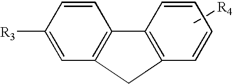

| | R₁ | R₂ | Ki, nM | | R₃ | R₄ | Ki, nM |
|---|---|---|---|---|---|---|---|
| 1a- | H | 2-NH₂ | >1,000 | 2a- | H | 2-NMe₂ | >1,000 |
| VIII | | | | VIII | | | |
| 1b- | H | 3-NH₂ | >1,000 | 2b- | H | 3-NMe₂ | 23.5 ±6.0 |
| VIII | | | | VIII | | | |
| 1c- | H | 4-NH₂ | >1,000 | 2c- | H | 4-NMe₂ | >1,000 |
| VIII | | | | VIII | | | |
| 1d- | Br | 2-NH₂ | 56 ± 2 | 2d- | Br | 2-NMe₂ | 0.85 ±0.1 |
| VIII | | | | VIII | | | |
| 1e- | NH₂ | 2-NH₂ | >1,000 | 2e- | NMe₂ | 2-NMe₂ | 15.4 ±5.0 |
| VIII | | | | VIII | | | |
| 1f- | I | 2-NH₂ | — | 2f- | I | 2-NMe₂ | 0.92 ±0.10 |
| VIII | | | | VIII | | | |

Scheme 26

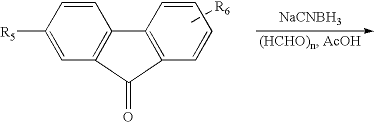

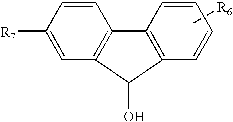

| R₅ | R₆ | | R₇ | R₆ | Ki, nM |
|---|---|---|---|---|---|
| H | 2-NH₂ | 3a-VIII | H | 2-NMe₂ | >1,000 |
| H | 4-NH₂ | 3b-VIII | H | 4-NMe₂ | >1,000 |
| Br | 2-NH₂ | 3c-VIII | Br | 2-NMe₂ | 88 ± 4 |
| H | 2-NH₂ and 3-Br | 3d-VIII | H | 2-NMe₂ and 3-Br | >1,000 |

Scheme 27

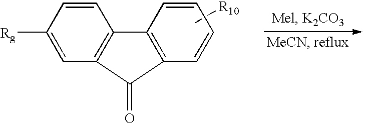

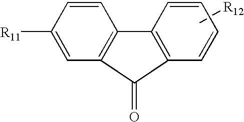

| R₉ | R₁₀ | | R₁₁ | R₁₂ | Ki, nM |
|---|---|---|---|---|---|
| H | 2-NH₂ | 4a-VIII | H | 2-NMe₂ | >1,000 |
| H | 3-NH₂ | 4b-VIII | H | 3-NMe₂ | >1,000 |
| H | 4-NH₂ | 4c-VIII | H | 4-NMe₂ | >1,000 |
| Br | 2-NH₂ | 4d-VIII | Br | 2-NMe₂ | 16.5 ± 4 |

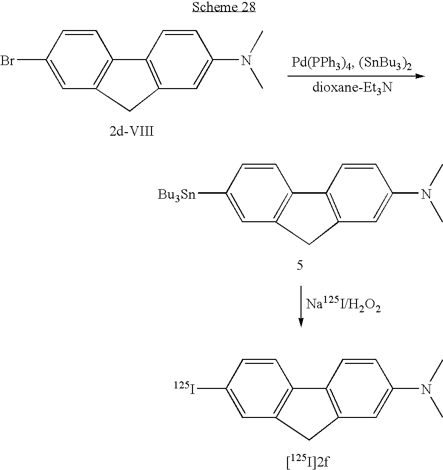

Scheme 29 and 30 depict a synthetic route for preparing biphenyl compounds of Formula VII.

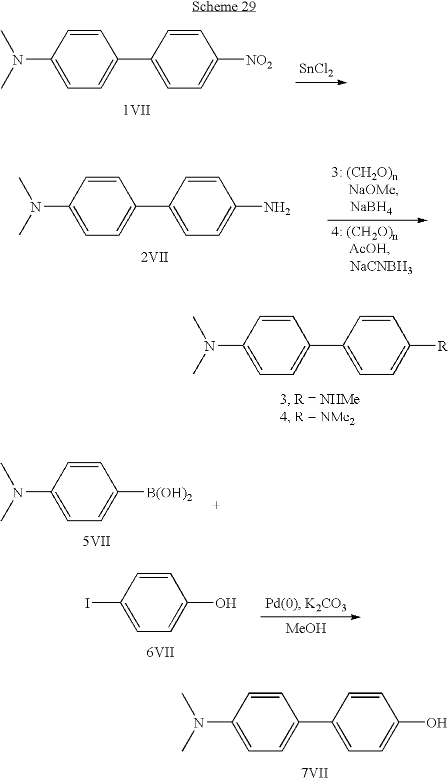

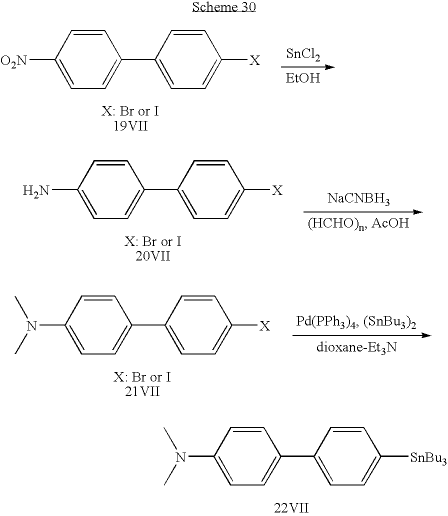

One of the key prerequisites for a brain amyloid inhibiting agent is the ability to cross the intact blood-brain barrier after a bolus iv injection. The compounds of the present invention possess a core ring system comprised of various substituted, fused 5- and 6-member aromatic rings. Several compounds of this invention contain a benzothiazole core and are derivatives of thioflavins. These compounds contain no quaternary ammonium ion, therefore, they are relatively small in size, neutral and lipophilic (Partition Coefficient=70 and 312 for 3 and 6a, respectively).

To test the permeability through the intact blood-brain barrier several compounds of Formula I or III were injected into normal mice. Initial brain uptake of 3 and 6a in mice after an iv injection was 0.67 and 1.50% dose/organ, respectively (see Table 2). The brain uptake peaked at 60 min for both compounds with a maximum brain uptake of 1.57 and 1.89% dose/organ, respectively. The blood levels are relatively low throughout the time points evaluated. For this series of ligands, specific uptake in the brain is relatively high and the retention in the brain is long.

The following examples are illustrative, but not limiting, of the method and compositions of the present invention. Other suitable modifications and adaptations of the variety of conditions and parameters normally encountered and obvious to those skilled in the art are within the spirit and scope of the invention.

### EXAMPLE 1

### Diethyl 2-iodobenzylphosphonate (11-I)

A mixture of 2-iodobenzyl bromide 10-I (5 g, 16.84 mmol) and triethyl phosphite (3.3 g, 20 mmol) was stirred at 160° C. After 4 h, the mixture was cooled to room temperature. The residue was subjected to flash chromatography (EtOAc: Hex, 1:4), and gave 2.3 g of 11-I (39%). ¹H NMR (200 MHz, CDCl₃): δ 1.24 (t, J=7.04 Hz, 6H), 3.40 (d, J=22.00 Hz, 2H), 4.03 (m, 4H), 6.91 (m, 1H), 7.32 (m, 1H), 7.44 (m, 1H), 7.82 (m, 1H); ¹³C NMR (50 MHz, CDCl₃): δ 16.27 (J=6.00 Hz), 38.31 (J=137.50 Hz), 62.16 (J=6.70 Hz), 101.16 (J=9.45 Hz), 128.23 (J=3.35 Hz), 128.45 (J=3.55 Hz), 130.60 (J=5.10 Hz), 135.36 (J=8.80 Hz), 139.60 (J=2.85 Hz).

### EXAMPLE 2

### (E)-2′-Iodo-N,N-dimethyl-4-stilbenamine (4-I)

To a mixture of NaH (2 mmol, 80% suspension in oil), and 3-iodobenzylphosphonate 2-I (500 mg, 1.42 mmol) in 6 mL of THF at 80° C. under nitrogen atmosphere, was added dropwise 4-(dimethylamine)benzaldehyde (210 mg, 1.41 mmol). After overnight at room temperature, NH₄Cl solution (saturated, 5 mL) was added and the mixture was extracted with CH₂Cl₂ (3×30 mL). The combined organic extract was dried over Na₂SO₄ and evaporated to give (E)-2′-iodo-N,N-dimethyl-4-stilbenamine 11-I, which was purified by flash chromatography (EtOAc: Hex, 1:9) to give 3-I (330 mg, 67%). ¹H NMR (200 MHz, CDCl₃): δ 3.06 (s, 6H), 6.82 (m, 2H), 6.93-7.02 (m, 1H), 7.01 (d, J=15.98 Hz, 1H), 7.25 (d, J=15.99 Hz, 1H), 7.40 (m, 1H), 7.53-7.59 (m, 2H), 7.69 (dd, J=7.88 Hz, J=1.54 Hz, 1H), 7.95 (dd, J=7.92 Hz, J=1.20 Hz, 1H); ¹³C NMR (50 MHz, CDCl₃): δ 40.26, 100.20, 112.22, 125.12, 125.61, 127.83, 127.87, 127.97, 128.2, 131.66, 139.42, 140.84, 150.23; HRMS: m/z Calcd for C₁₆H₁₆IN, 349.0328; Found: 349.0342.

### EXAMPLE 3

### Diethyl 3-iodobenzylphosphonate (13-I)

A mixture of 3-iodobenzyl bromide 12-I (5 g, 16.84 mmol) and triethyl phosphite (3.3 g, 20 mmol) was stirred at 160° C. After 4 h, the mixture was cooled to room temperature. The residue was subjected to flash chromatography (EtOAc: Hex, 1:4), and gave 5.4 g of 13-I (91%). ¹H NMR (200 MHz, CDCl₃): δ 1.15 (t, J=7.05 Hz, 6H), 2.97 (d, J=21.65 Hz, 2H), 3.92 (m, 4H), 6.93 (t, J=7.76 Hz, 1H), 7.17 (m, 1H), 7.52 (m, 2H); ¹³C NMR (50 MHz, CDCl₃): δ 16.25 (J=5.95 Hz), 33.15 (J=137.60 Hz), 62.19 (J=6.70 Hz), 94.13 (J=3.50 Hz), 128.89 (J=6.35 Hz), 130.07 (J=3.00 Hz), 133.95 (J=9.10 Hz), 135.87 (J=3.55 Hz), 138.51 (J=6.65 Hz).

### EXAMPLE 4

### (E)-3′-Iodo-N,N-dimethyl-4-stilbenamine (5-I)

To a mixture of NaH (2 mmol, 80% suspension in oil), and 3-iodobenzylphosphonate 13-I (370 mg, 1.05 mmol) in 5 mL of THF at 80° C. under nitrogen atmosphere, was added dropwise 4-(dimethylamine)benzaldehyde (155 mg, 1.05 mmol). After overnight at room temperature, NH₄Cl solution (saturated, 5 mL) was added and the mixture was extracted with CH₂Cl₂ (3×20 mL). The combined organic extract was dried over Na₂SO₄ and evaporated to give (E)-3′-iodo-N,N-dimethyl-4-stilbenamine 5-I, which was purified by flash chromatography (EtOAc: Hex, 1:9) to give 3-I (209 mg, 57%). ¹H NMR (200 MHz, CDCl₃): δ 2.99 (s, 6H), 6.71 (m, 2H), 6.77 (d, J=16.41 Hz, 1H), 7.02 (d, J=16.22 Hz, 1H), 7.04 (t, J=7.8 Hz, 1H), 7.36-7.52 (m, 4H), 7.82 (s, 1H); ¹³C NMR (50 MHz, CDCl₃): δ 40.37, 94.78, 112.38, 112.53, 122.21, 127.76, 128.56, 130.19, 134.76, 135.34, 140.56, 150.36; HRMS: m/z Calcd for C₁₆H₁₆IN, 349.0328. Found: 349.0302.

### EXAMPLE 5

### Diethyl 4-iodobenzylphosphonate (15-I)

A mixture of 4-iodobenzyl bromide 14-I (5.2 g, 17.51 mmol) and triethyl phosphite (3.3 g, 20 mmol) was stirred at 160° C. After 4 h, the mixture was cooled to room temperature. The residue was subjected to flash chromatography (EtOAc: Hex, 1:4), and gave 3.27 g of 15-I (53%). ¹H NMR (200 MHz, CDCl₃): δ1.24 (t, J=7.04 Hz, 6H), 3.07 (d, J=21.72 Hz, 2H), 4.01 (m, 4H), 7.04 (m, 2H), 7.62 (m, 2H); ¹³C NMR (50 MHz, CDCl₃): δ 16.24 (J=5.90 Hz), 33.21 (J=137.55 Hz), 62.04 (J=6.70 Hz), 92.15 (J=4.80 Hz), 131.31 (J=9.10 Hz), 131.57 (J=6.55 Hz), 137.43 (J=2.95 Hz).

### EXAMPLE 6

### (E)-4′-Iodo-N,N-dimethyl-4-stilbenamine (6-I)

To a mixture of NaH (2 mmol, 80% suspension in oil) and 4-iodobenzylphosphonate 15-I (420 mg, 1.19 mmol) in 5 mL of THF at 80° C. under nitrogen atmosphere, was added dropwise 4-(dimethylamine)benzaldehyde (180 mg, 1.20 mmol). After overnight at room temperature, water (5 mL) was added. The solid formed was filtered and washed with ether to give crude 6-I which was purified by recrystallization with CH₂Cl₂/hexane to afford pure 6-I (156 mg, 38%). ¹H NMR (200 MHz, CDCl₃): δ 2.99 (s, 6H), 6.71 (d, J=8.60 Hz, 2H), 6.81 (d, J=16.65 Hz, 1H), 7.04 (d, J=16.12 Hz, 1H), 7.21 (d, J=8.15 Hz, 1H), 7.38 (d, J=8.59 Hz, 2H), 7.63 (d, J=8.28 Hz, 2H); ¹³C NMR (50 MHz, CDCl₃): δ 40.39, 91.32, 112.38, 123.04, 127.69, 127.73, 128.23, 129.65, 137.55, 137.77, 150.29; HRMS: m/z Calcd for C₁₆H₁₆IN, 349.0328; Found: 349.0288.

### EXAMPLE 7

### (E)-4′-Iodo-4-O-methoxystilbenol (8-I)

To a mixture of NaH (2 mmol, 80% suspension in oil), and 3-iodobenzylphosphonate 13-I (450 mg, 1.27 mmol) in 7 mL of THF at 80° C. under nitrogen atmosphere, was added dropwise p-anisaldehyde (172 mg, 1.27 mmol). After 3 days at room temperature, NH₄Cl solution (saturated, 5 mL) was added and the mixture was extracted with CH₂Cl₂ (3×30 mL). The combined organic extract was dried over Na₂SO₄, evaporated and purified by flash chromatography (EtOAc: Hex, 1:9) to give (E)-1-iodo-3-[2-(4-methoxyphenyl)ethenyl]benzene 8-I (400 mg, 90%). ¹H NMR (200 MHz, CDCl₃): δ 3.84 (s, 3H), 6.84 (d, J=16.29 Hz, 1H), 6.90 (m, 2H), 7.05 (d, J=16.30 Hz, 1H), 7.07 (t, J=7.8 Hz, 1H), 7.42-7.56 (m, 4H), 7.85 (s, 1H); ¹³C NMR (50 MHz, CDCl₃): δ55.32, 94.76, 114.20, 124.85, 125.48, 127.88, 129.58, 129.62, 130.25, 135.00, 135.91, 139.97, 159.62; HRMS: m/z Calcd for C₁₅H₁₃IO: 336.0011; Found: 336.0006.

### EXAMPLE 8

### (E)-3′-Iodo-4-stilbenol (9-I)

To a solution of 8-I (350 mg, 1.00 mmol) in CH₂Cl₂ (200 mL) was added BBr₃ (10 mL, 1M in hexane) dropwise at −78° C. in a dry ice-acetone bath. The mixture was allowed to warm up to room temperature. Water was added while the reaction mixture was cooled at 0° C. in an ice bath. The mixture was extracted with CH₂Cl₂. The organic phase was dried and filtered. The filtrate was purified by flash chromatography (EtOAc: Hex, 1:9) to give 9-I (296 mg, 92%). ¹H NMR (200 MHz, CDCl₃): δ 4.81 (s, 1H), 6.83 (d, J=16.17 Hz, 1H), 6.84 (m, 2H), 7.03 (d, J=16.32 Hz, 1H), 7.06 (t, J=7.8 Hz, 1H), 7.36-7.57 (m, 4H), 7.84 (s, 1H); ¹³C NMR (50 MHz, CDCl₃): δ 94.75, 115.67, 124.96, 125.49, 128.09, 129.48, 129.87, 130.25, 135.01, 135.96, 139.90, 155.53; HRMS: m/z Calcd for C₁₄H₁₁IO: 321.9855; Found: 321.9840.

### EXAMPLE 9

### Diethyl, 4-fluorobenzylphosphonate (17-I)

A mixture of 4-fluorobenzyl bromide 16-I (1.89 g, 10 mmol) and triethyl phosphite (1.66 g, 10 mmol) was stirred at 170° C. for 4 h. The mixture was cooled to room temperature. and the residue was subjected to flash chromatography (EtOAc:Hex, 1:4) to gave 1.4 g of 17-I (57%).

¹H NMR (200 MHz, CDCl₃): δ 1.23 (t, J=7.1 Hz, 6H), 3.10 (d, J=21.4 Hz, 2H), 3.92 (q, J=7.1 Hz, 4H), 7.02 (m, 2H), 7.25 (m, 2H).

### EXAMPLE 10

### (E)-4-Fluoro-4′-dimethylamino-stilbene (7-I)

To a mixture of phosphate 17-I (246 mg, 1 mmol) and 4-dimethylaminobenzaldehyde (149 mg, 1 mmol) in DMF (2 mL) was added KO^{t}Bu (224 mg, 2 mmol) in portions in solid form at RT. The resulting mixture was stirred at RT overnight. Water (10 mL) was added The solid was collected by suction and washed with water, dried to give 190 mg of product (80%). ¹H NMR (200 MHz, CDCl₃): δ 2.99 (s, 6H), 6.71 (d, J=8.9 Hz, 2H), 6.85 (d, J=16.3 Hz, 1H), 7.01 (t, J=8.7 Hz, 2H), 7.40 (d, J=9.0 Hz, 2H), 7.43 (m, 2H); ¹³C NMR (200 MHz, CDCl₃): δ41.00, 113.01, 115.78, 116.21, 123.76, 126.18, 127.83, 127.99, 128.05, 129.19, 134.91, 150.72, 164.81.

### EXAMPLE 11

### (E)-3-Tributylstannyl-4′-dimethylamino-stilbene (18-I)

A mixture of 5-I (139 mg, 0.38 mmol), bis-(tributylyltin) (0.4 mL) and Pd(Ph₃P)₄ (30 mg) in a mixed solvent (20 mL, dioxane:triethylamine, 3:1) was stirred at 90° C. overnight. Solvent was removed and the residue was purified by PTLC (Hex:EtOAc, 2:1) to give 35 mg of product (18%, not optimized yield). ¹H NMR (200 MHz, CDCl₃): δ 0.94 (t, J=7.2 Hz, 9H), 1.08-1.66 (m, 18H), 3.01 (s, 6H), 6.75 (m, 2H), 6.94 (d, J=16.3 Hz, 1H), 7.08 (d, J=16.3 Hz, 1H), 7.25-7.57 (m, 6H); ¹³C NMR (50 MHz, CDCl₃): δ 9.56, 13.67, 27.37, 29.10, 40.45, 112.45, 124.84, 125.44, 125.98, 127.51, 128.01, 128.51, 134.36, 134.89, 137.41, 142.09, 150.06; HRMS: m/z Calcd for C₂₈H₄₄NSn (MH⁺): 514.2496; Found: 514.2512.

### EXAMPLE 12

### Preparation of Radioiodinated Ligand

The desired ¹²⁵I-labeled compound was prepared using iododestannylation reactions with tributyltin precursor of 5-I. Hydrogen peroxide (50 μL, 3% w/v) was added to a mixture of 50 μL of the corresponding tributyltin precursor, 18-I, (1 μg/μL EtOH), 50 μL of 1N HCl and [¹²⁵I]NaI (1-5 mCi) in a closed vial. The reaction was allowed to proceed for 10 min at room temperature and terminated by addition of 100 μL of sat. NaHSO₃. The reaction mixture was extracted with ethyl acetate (3×1 mL) after neutralization with saturated sodium bicarbonate solution. The combined extracts were evaporated to dryness. The residue was dissolved in 100 μL of EtOH and purified by HPLC using a reversed phase column (Waters C-18 ubondpad, 3.9×300 mm) with an isocratic solvent of 80% acetonitrile-20% of buffer, 3,3-dimethylglutaric acid (5 mM, pH 7.0) in a flow rate of 0.8 mL/min. The desired fractions containing the product were collected, condensed and re-extracted with ethyl acetate. The no-carrier-added product was evaporated to dryness and re-dissolved in 100% EtOH (1 μCi/μL), The final ¹²⁵I probe, with a specific activity of 2,200 Ci/mmole and a greater than 95% radiochemical purity, was stored at −20° C. up to 6 weeks for in vitro binding studies.

### EXAMPLE 13

### Binding Assays Using Aggregated Aβ(1-40) Peptide in Solution

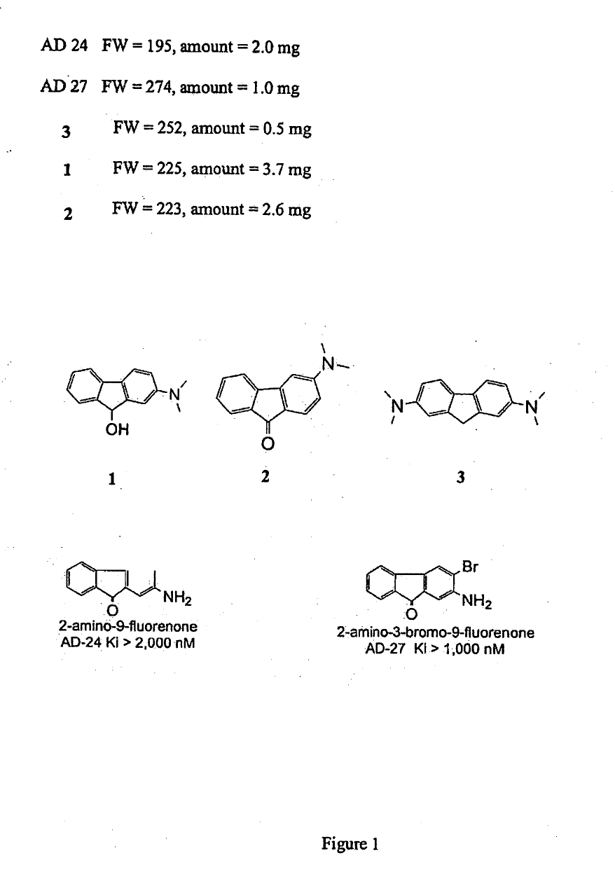

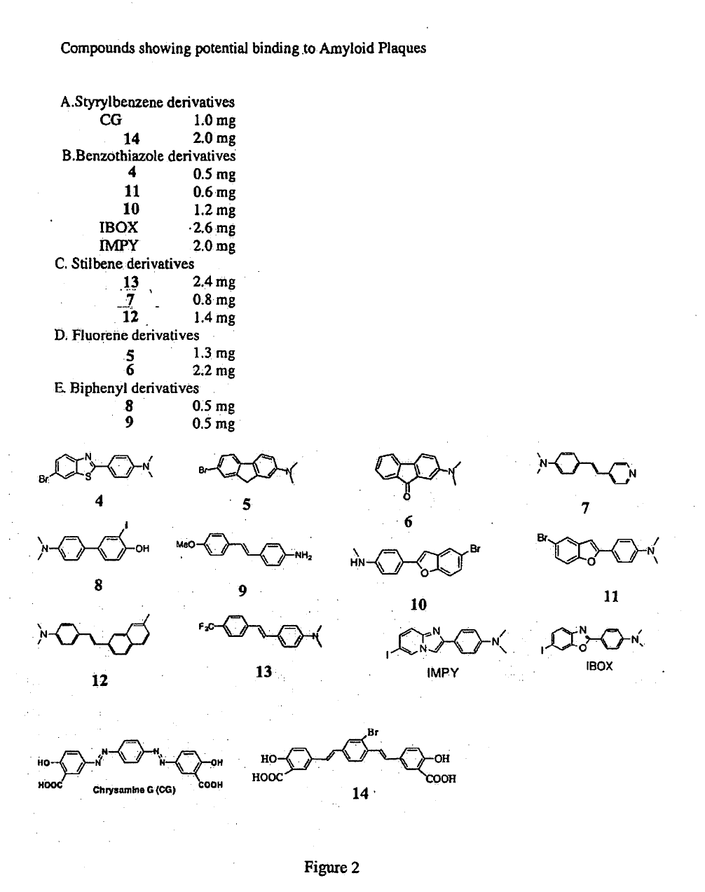

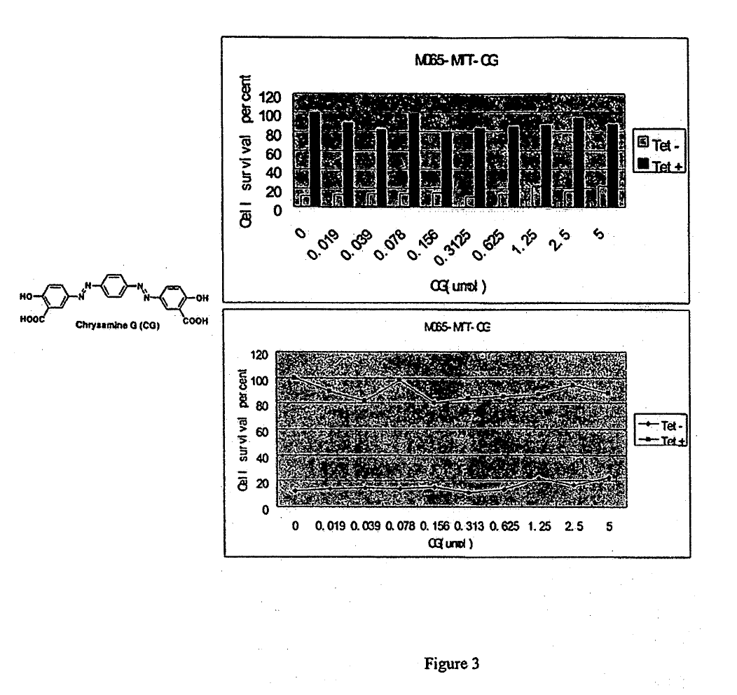

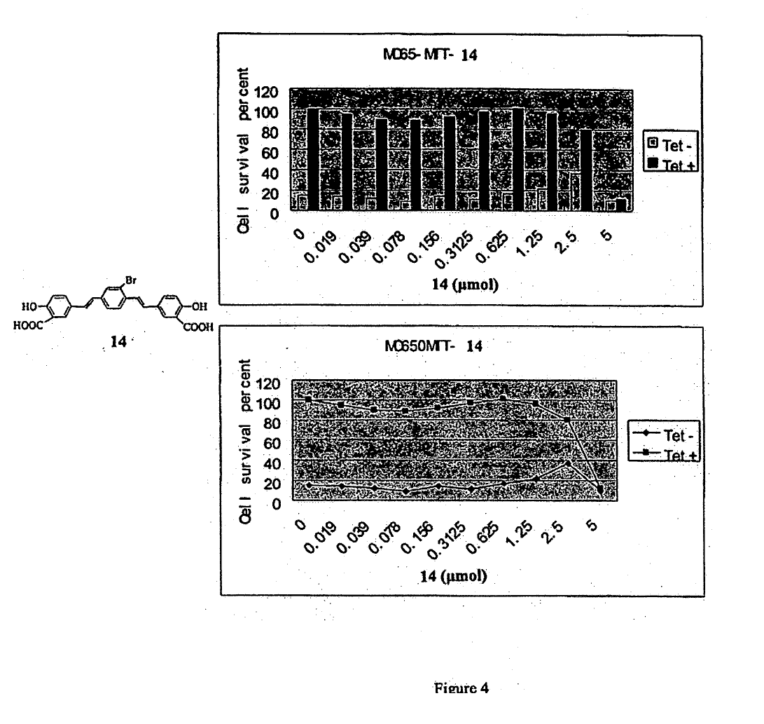

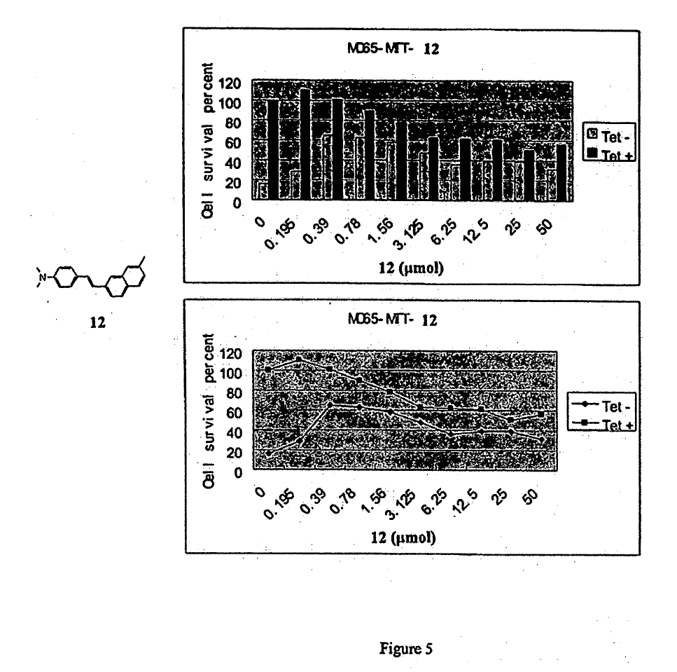

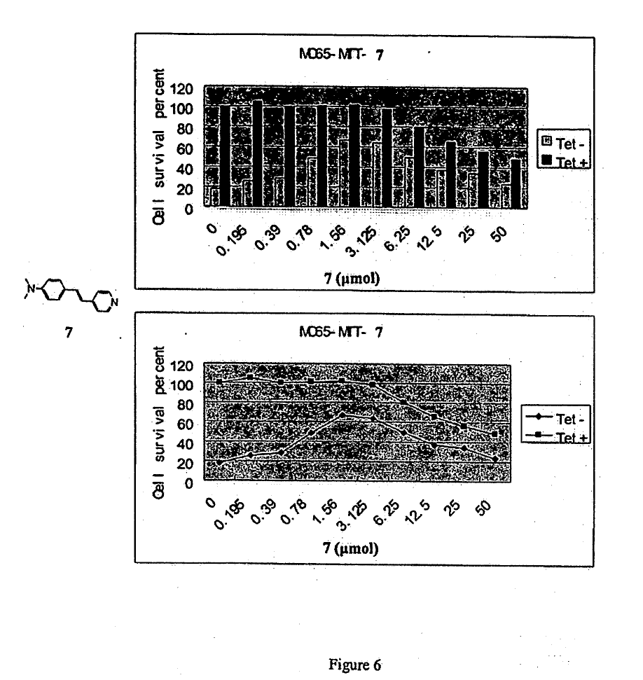

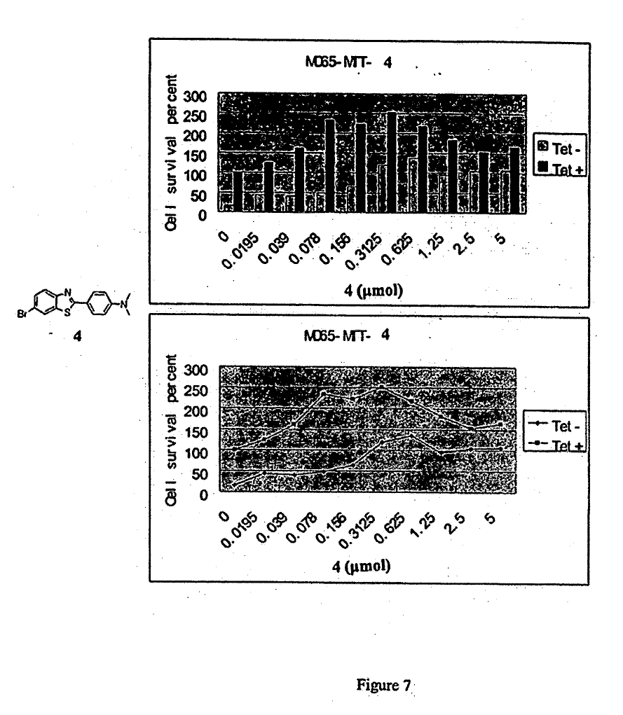

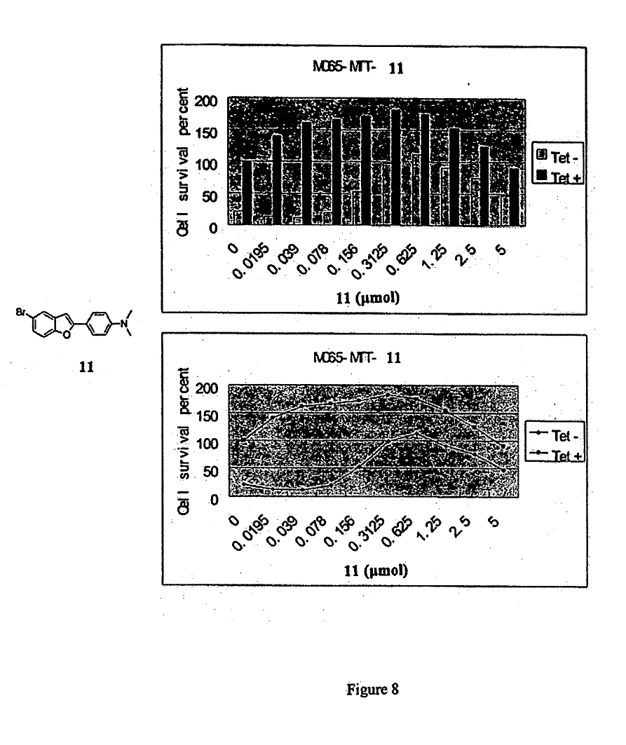

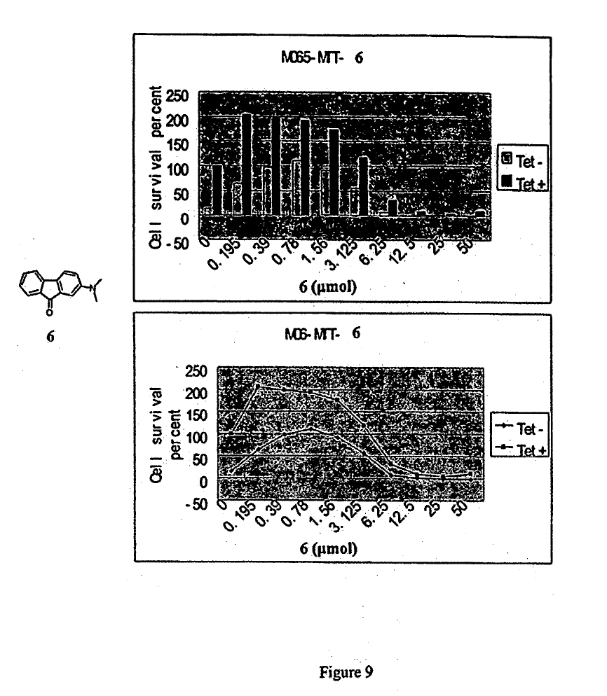

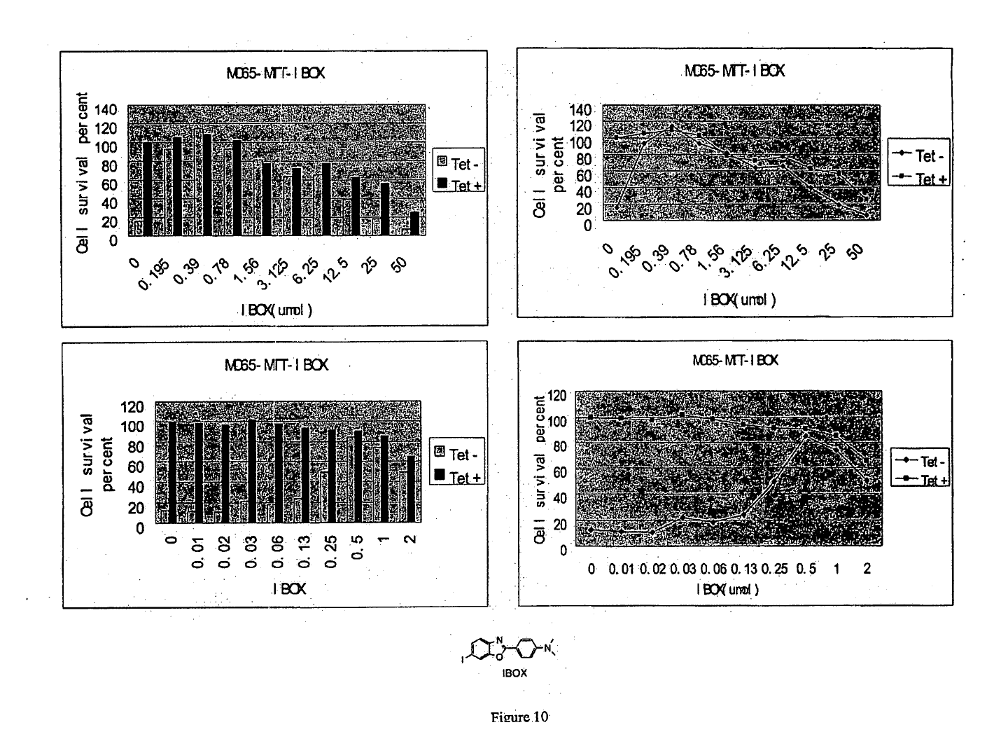

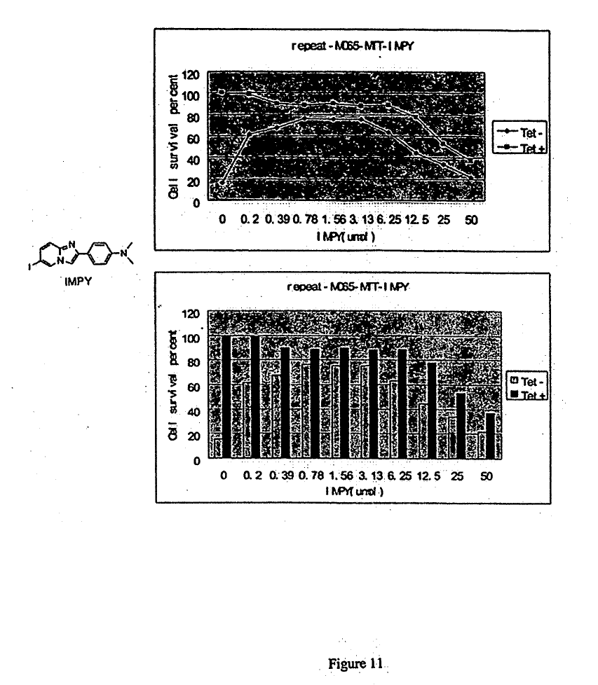

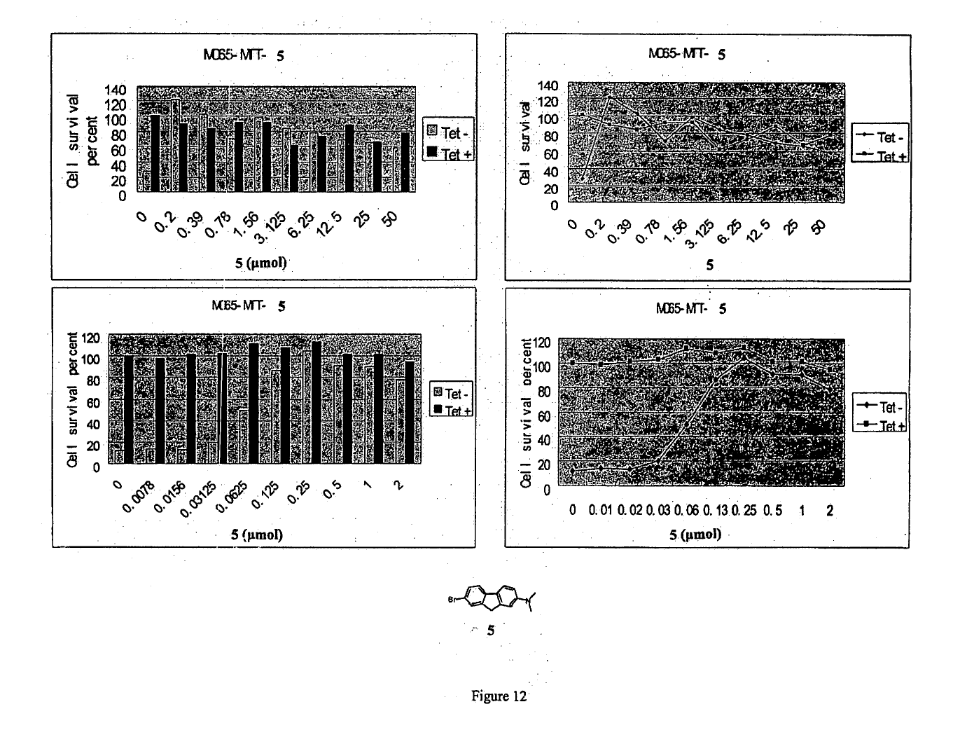

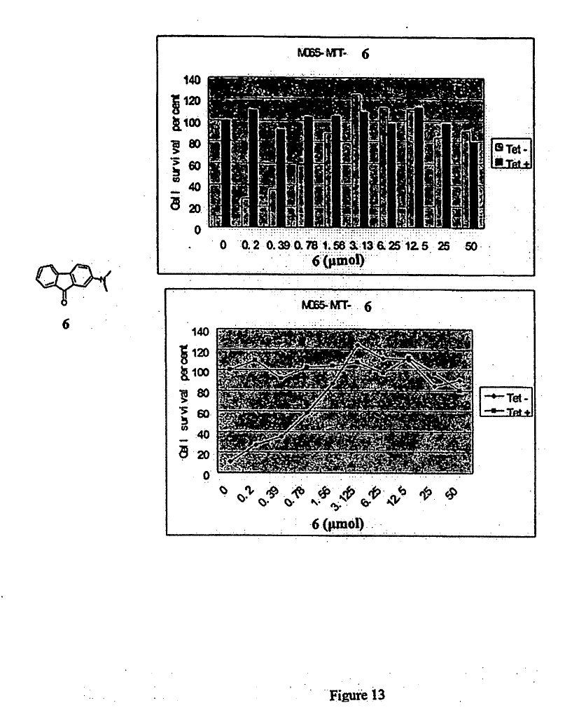

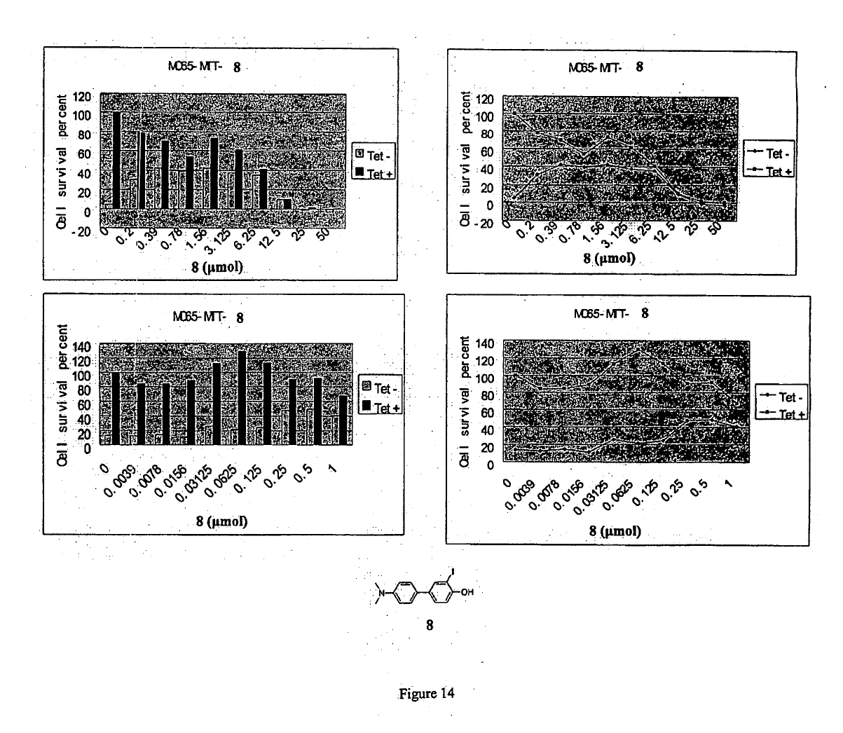

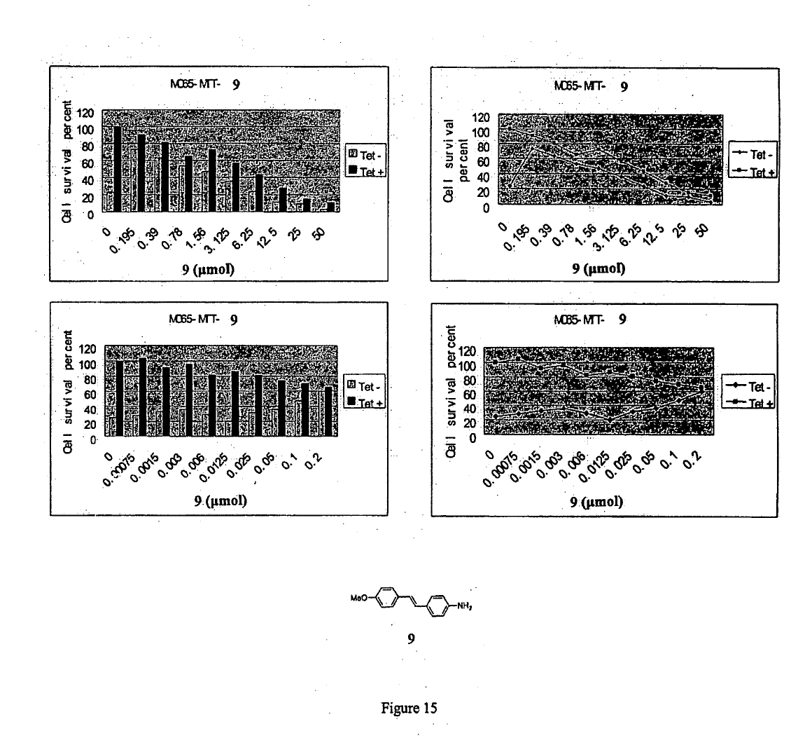

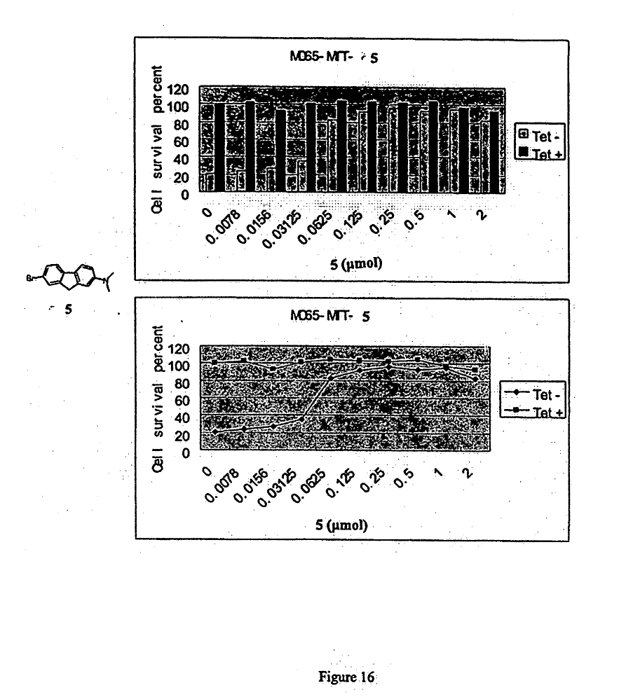

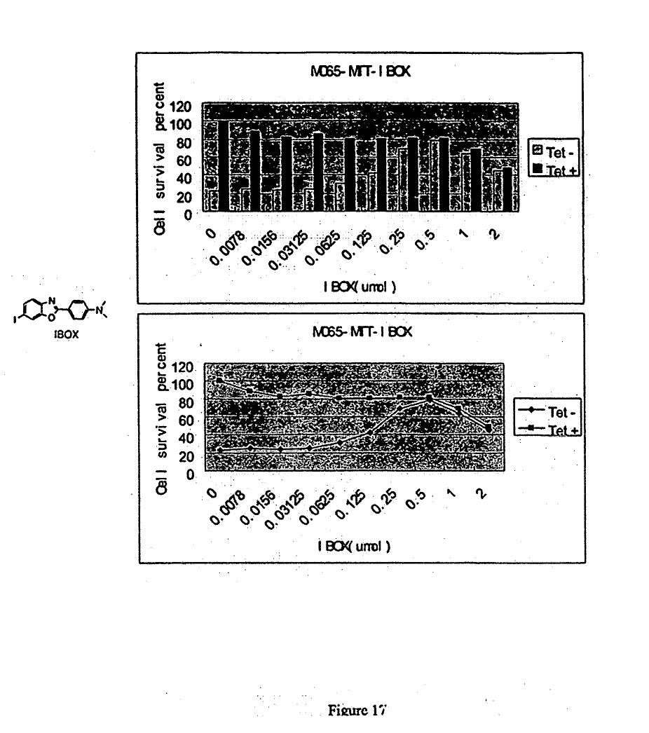

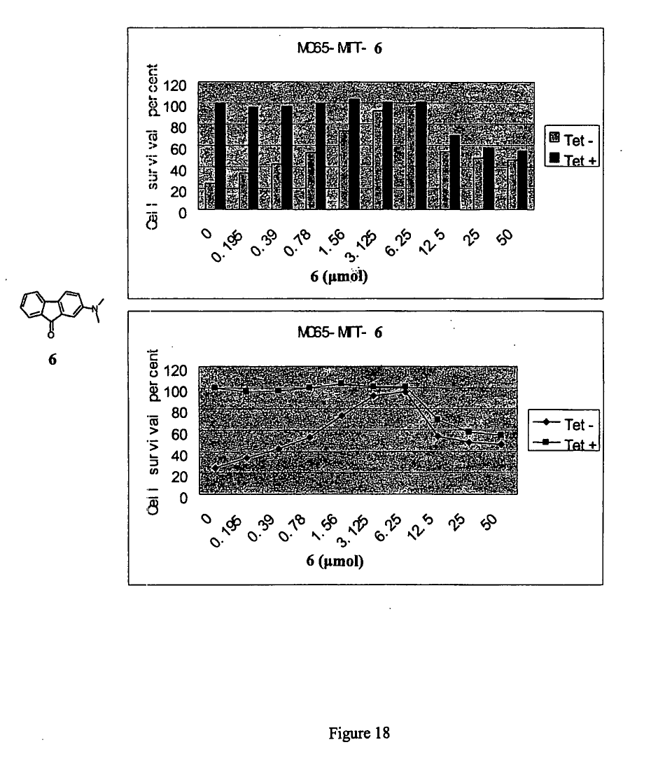

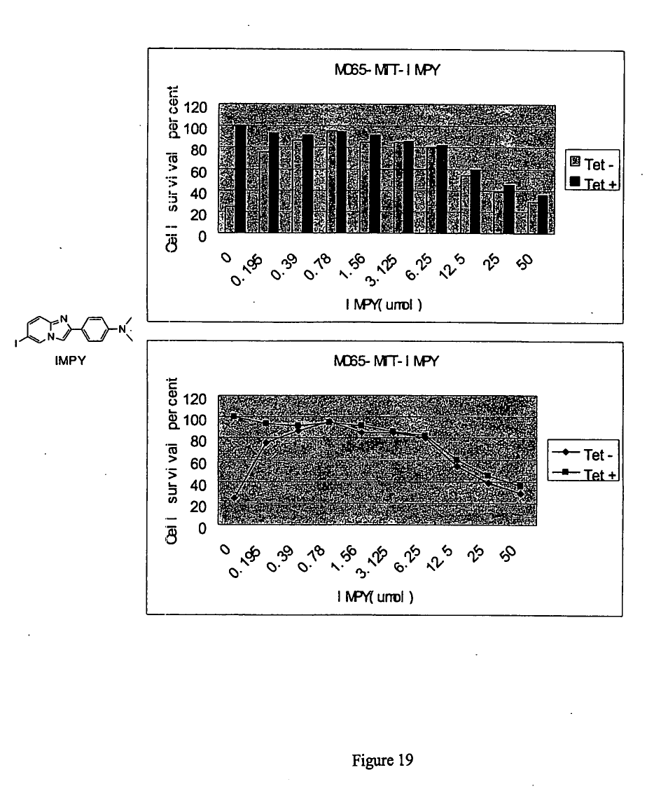

The solid forms of peptides Aβ(1-40) was purchased from Bachem (King of Prussia, Pa.). Peptide aggregation was carried out by gently dissolving the peptide (0.5 mg/mL) in a buffer solution (pH 7.4) containing 10 mM sodium phosphate and 1 mM EDTA. The solution was incubated at 37° C. for 36-42 h with gentle and constant shaking. Binding studies were carried out in 12×75 mm borosilicate glass tubes according to the procedure described¹. Aggregated fibrils (10-50 nM in the final assay mixture) were added to the mixture containing 50 μl of radioligands (0.01-0.5 nM in 40% EtOH) and 10% EtOH in a final volume of 1 mL for saturation studies. The final concentration of EtOH was 10%. Nonspecific binding was defined in the presence of 2 μM thioflavin T. For inhibition studies, 1 mL of the reaction mixture contained 40 μl of inhibitors (10⁻⁵-10⁻¹⁰ M in 10% EtOH) and 0.05 nM radiotracer in 40% EtOH. The mixture was incubated at room temperature for 3 h and the bound and the free radioactivities were separated by vacuum filtration through Whatman GF/B filters using a Brandel M-24R cell harvester followed by 2×3 mL washes of 10% ethanol at room temperature. Filters containing the bound I-125 ligand were counted in a gamma counter (Packard 5000) with 70% counting efficiency. Under the assay conditions, the percent of the specifically bound fraction was less than 20% of the total radioactivity. The results of saturation and inhibition experiments were subjected to nonlinear regression analysis using software EBDA² by which K_{d} and Kᵢ values were calculated. Values for (Kᵢ, nM) are the mean ±SEM of three independent experiments, each in duplicate. Additional Kᵢ values for compounds of Formula I are provided in FIGS. 1 and 2.

In in vitro binding assays using pre-formed Aβ aggregates of synthetic peptides and [¹²⁵I]TZDM as the ligand, these novel stilbenes showed exceedingly high binding affinity (2-40 nM) to the TZ sites, while the affinity towards SB sites was very low (>1,000 nM). It is evident that the stilbenes containing an electron donating groups, such as dimethylamino-, —OH or —OMe group, showed excellent binding affinity to Aβ aggregates. Benzothiazole ring appears to be unnecessary for binding at the TZ binding sites of Aβ aggregates. This information is of paramount importance because it reduces the molecular size (molecular weight of TZDM and 1-I was 380 and 349, respectively) required for binding to the TZ sites; as such it significantly enhances the flexibility on designing new ligands. The idoinated stilbenes, such as 2-I and 5-I, represent a structural simplicity, which suggests minimum requirements for binding the Aβ aggregates may be three: 1) two benzene rings separated by a vinyl group. 2) one of the aromatic ring contains a electronic negative group, dimethylamino-, —OH or —OMe group. 3) there appears to be a bulk tolerance for substitution on the second aromatic ring. To characterize the compounds further, radioactive iodinated ligand, [¹²⁵I]2-I, was prepared by converting the corresponding tributyltin derivative in the presence of Na[¹²⁵I]I and hydrogen peroxide, by which the no-carrier added product was obtained in excellent yield (radiochemical purity >95%). The direct binding assay showed that the new evaluation of postmortem AD brain sections with [¹²⁵I]2-I suggested that the novel ligand, as expected, labeled Aβ plaques.

### EXAMPLE 14

### In Vivo Biodistribution of New Probes in Normal Mice

While under ether anesthesia, 0.15 mL of a saline solution containing the labeled agent (5-10 μCi) was injected directly into the tail vein of ICR mice (2-3 month-old, average weight 20-30 g). The mice were sacrificed by cardiac excision at various time points post injection. The organs of interest were removed and weighed, and the radioactivity was counted with an automatic gamma counter (Packard 5000). The percentage dose per organ was calculated by a comparison of the tissue counts to suitably diluted aliquots of the injected material. Total activities of blood and muscle were calculated under the assumption that they were 7% and 40% of the total body weight, respectively.

In vivo biodistribution study of [¹²⁵I]2-I in normal mice after an iv injection suggested good brain penetration. The brain uptake was 0.84, 1.08, 0.91, and 0.54% dose/organ, at 2, 30, 60 and 120 minutes after injection (the blood levels was relatively low 5.2-3.6% dose/organ at all of the time points). The radioactive ligand's binding to the aggregates of Aβ₁₋₄₀ is saturable and the K_{d} was 0.2 nM.

### EXAMPLE 15

### 2-(Dimethylamino)fluorene (2a-VIII)

To a stirred mixture of 2-aminofluorene (119 mg, 0.66 mmol) and papraformaldehyde (300 mg, 10 mmol) in 5 ml of AcOH at room temperature was added in one portion of NaCNBH₃ (300 mg, 4.8 mmol). The resulting mixture was stirred at room temperature for 18 h, then carefully poured into 25% aq. NaOH and ice chips to make strongly alkaline (pH 11) and extracted with methylene chloride. The combined extracts were dried, filtered, and concentrated in vacuo. The residue was subjected to flash chromatography (EtOAc:Hex, 1:4). and gave 129 mg of 2-(dimethylamino)fluorene (94%). ¹H NMR (200 MHz, CDCl₃): δ 3.09 (s, 6H), 7.00 (d, J=7.55 Hz, 1H), 7.33-7.38 (m, 2H), 7.61-7.96 (m, 5H), 8.11 (d, J=8.40 Hz, 1H); ¹³C NMR (50 MHz, CDCl₃): δ 45.43, 114.04, 119.74, 120.60, 120.98, 121.30, 123.99, 125.34, 126.20, 126.45, 127.40, 130.49, 134.03, 137.26, 139.08, 139.39, 151.83.

### EXAMPLE 16

### 3-(Dimethylamino)fluorene (2b-VIII)

The same reaction as described above for preparing 2a-VIII was employed, and 2b-VIII was obtained in 93% from 3-aminofluorene. ¹H NMR (200 MHz, CDCl₃): δ 3.05 (s, 6H), 3.89 (s, 2H), 6.82 (dd, J=8.44 Hz, J=2.35 Hz, 1H), 6.99 (s, 1H), 7.18-7.25 (m, 1H), 7.36 (t, J=7.30 Hz, 1H), 7.51 (d, J=7.31 Hz, 1H), 7.68 (d, J=8.44 Hz, 2H); ¹³C NMR (50 MHz, CDCl₃): δ 37.14, 41.04, 109.28, 111.67, 118.52, 120.43, 124.71, 124.79, 126.64, 131.12, 142.33, 142.43, 145.02, 150.33; Anal. Calcd for C₁₅H₁₅N: C, 86.08; H, 7.22. Found: C, 86.46; H, 6.89.

### EXAMPLE 17

### 4-(Dimethylamino)fluorene (2c-VIII)

The same reaction as described above for preparing 2a-VIII was employed, and 2c-VIII was obtained in 94% from 4-aminofluorene. ¹H NMR (200 MHz, CDCl₃): δ 3.01 (s, 6H), 4.01 (s, 2H), 6.96 (d, J=7.78 Hz, 1H), 7.33-7.54 (m, 4H), 7.63 (d, J=6.99 Hz, 5H), 7.85 (d, J=6.96 Hz, 1H); ¹³C NMR (50 MHz, CDCl₃): δ 36.99, 43.18, 113.26, 115.20, 119.90, 124.72, 126.62, 128.05, 133.73, 141.90, 143.20, 150.40.

### EXAMPLE 18

### 2-Dimethylamino-7-bromofluorene (2d-VIII)

The same reaction as described above for preparing 2a-VIII was employed, and 2d-VIII was obtained in 95% from 2-amino-7-bromofluorene. ¹H NMR (200 MHz, CDCl₃): δ 3.02 (s, 6H), 3.80 (s, 2H), 6.76 (dd, J=8.47 Hz, J=2.33 Hz, 1H), 6.90 (s, 1H), 7.56-7.61 (m, 4H); ¹³C NMR (50 MHz, CDCl₃): δ 36.91, 40.90, 108.94, 111.66, 118.14, 119.64, 120.53, 127.84, 129.63, 129.85, 141.44, 144.34, 144.73, 150.50; HRMS: m/z Calcd for C₁₅H₁₄BrN: 287.0301; Found: 287.0282; Anal. Calcd for C₁₅H₁₄BrN: C, 62.52; H, 4.90. Found: C, 62.46; H, 4.90.

### EXAMPLE 19

### 2,7-Bis(dimethylamine)fluorene (2e-VIII)

The same reaction as described above for preparing 2a was employed, and 2e was obtained in 61% from 2,7-diaminofluorene. ¹H NMR (200 MHz, CDCl₃): δ 2.98 (s, 6H), 3.81 (s, 2H), 6.75 (dd, J=2.41 Hz, J=8.39 Hz, 2H), 6.94 (s, 2H), 7.50 (d, J=8.37 Hz, 2H); ¹³C NMR (50 MHz, CDCl₃): δ 37.30, 41.30, 109.90, 111.82, 119.00, 132.12, 143.97, 149.23; HRMS: m/z Calcd for C₁₇H₂₀N₂: 252.1626; Found: 252.1618.

### EXAMPLE 20

### 2-Dimethylamino-7-iodofluorene (2f-VIII)

The same reaction as described above for preparing 2a-VIII was employed, and 2f-VIII was obtained in 93% from 2-amino-7-iodofluorene, which is readily prepared by reduction of 2-nitro-7-iodofluorene by SnCl₂. ¹H NMR (200 MHz, CDCl₃): δ 3.01 (s, 6H), 3.81 (s, 2H), 6.76 (dd, J=8.47 Hz, J=2.33 Hz, 1H), 690 (s, 1H), 7.56-7.61 (m, 4H); ¹³C NMR (50 MHz, CDCl₃): δ36.91, 40.90, 108.94, 111.66, 118.14, 119.64, 120.53, 127.84, 129.63, 129.85, 141.44, 144.34, 144.73, 150.50; HRMS: m/z Calcd for C₁₅H₁₄BrN: 335.0171; Found: 335.0184.

### EXAMPLE 21

### 2-Dimethylamino-9-hydroxyfluorene (3a-VIII)

To a stirred mixture of 2-amino-9-fluorenone (104 mg, 0.53 mmol) and papraformaldehyde (200 mg, 6 mmol) in 5 ml of AcOH at room temperature was added in one portion of NaCNBH₃ (200 mg, 3.2 mmol). The resulting mixture was stirred at room temperature for 18 h, then carefully poured into 25% aq. NaOH and ice chips to make strongly alkaline (pH 11) and extracted with methylene chloride. The combine extracts were dried, filtered, and concentrated in vacuo. The residue was subjected to flash chromatography (EtOAc:Hex, 1:4) and gave 100 mg of 2-dimethylamino-9-fluorenone (84%). ¹H NMR (200 MHz, CDCl₃): δ 2.08 (br s, 1H), 2.98 (s, 6H), 5.47 (br s, 1H), 6.68 (dd, J=8.41 Hz, J=2.45 Hz, 1H), 7.01 (d, J=2.35 Hz, 1H), 7.18 (dt, J=1.17 Hz, J=7.35 Hz, 1H), 7.32 (dt, J=1.02 Hz, J=7.33 Hz, 1H), 7.44-7.57 (m, 3H); ¹³C NMR (50 MHz, CDCl₃): δ 40.83, 75.35, 109.32, 112.91, 118.52, 120.60, 124.78, 125.69, 128.64, 128.90, 140.78, 144.92, 147.35, 150.83.

### EXAMPLE 22

### 4-Dimethylamino-9-hydroxyfluorene (3b-VIII)

The same reaction as described above for preparing 3a-VIII was employed, and 3b-VIII was obtained in 99% from 4-amino-9-fluorenone. ¹H NMR (200 MHz, CDCl₃): δ 2.35 (d, J=9.59 Hz, 1H), 2.83 (s, 6H), 5.46 (d, J=9.44 Hz, 1H), 7.06-7.10 (m, 1H), 7.25-7.44 (m, 4H), 7.58 (d, J=7.26 Hz, 1H), 7.99 (d, J=7.61 Hz, 1H); ¹³C NMR (50 MHz, CDCl₃): δ 44.24, 75.01, 118.41, 118.98, 123.74, 124.33, 126.70, 128.38, 128.78, 132.13, 139.41, 145.61, 147.86, 149.95.

### EXAMPLE 23

### 2-Dimethylamino-7-bromo-9-hydroxyfluorene (3c-VIII)

The same reaction as described above for preparing 3a-VIII was employed, and 3c-VIII was obtained in 87% from 2-amino-7-bromo-9-fluorenone. ¹H NMR (200 MHz, CDCl₃): δ 1.89 (d, J=10.07 Hz, 1H), 3.01 (s, 1H), 5.46 (d, J=9.06 Hz, 1H), 6.71 (dd, J=8.45 Hz, J=2.46 Hz, 1H), 7.00 (d, J=2.41 Hz, 1H), 7.31-7.47 (m, 3H), 7.67 (t, J=1.01 Hz, 1H); ¹³C NMR (50 MHz, CDCl₃): δ40.63, 74.98, 108.89, 112.82, 118.95, 119.70, 120.68, 127.27, 128.07, 131.78, 139.69, 146.76, 146.98, 150.97; HRMS: m/z Calcd for C₁₅H₁₄BrNO: 303.0259; Found: 303.0242; Anal. Calcd for C₁₅H₁₄BrNO: C, 59.23; H, 4.64. Found: C, 59.41; H, 4.60.

### EXAMPLE 24

### 2-Dimethylamino-3-bromo-9-hydroxyfluorene (3d-VIII)

The same reaction as described above for preparing 3a-VIII was employed, and 3d-VIII was obtained in 85% from 2-amino-3-bromo-9-fluorenone. ¹H NMR (200 MHz, CDCl₃): δ 2.04 (d, J=9.63 Hz, 1H), 2.83 (s, 6H), 5.44 (d, J=9.78 Hz, 1H), 7.25-7.39 (m, 3H), 7.48-7.59 (m, 2H), 7.78 (s, 1H); ¹³C NMR (50 MHz, CDCl₃): δ44.39, 74.88, 117.21, 119.57, 119.93, 125.03, 125.35, 127.42, 129.18, 135.85, 138.91, 145.53, 145.84, 151.60.

### EXAMPLE 25

### 2-Dimethylamino-9-fluorenone (4a-VIII)

To a stirred mixture of 2-amino-9-fluorenone (315 mg, 1.6 mmol) and potassium carbonate (300 mg) in 5 ml of acetonitrile was added in one portion of iodomethane (0.5 ml). After overnight at reflux, NH₄Cl solution (saturated, 5 mL) is added and the mixture is extracted with CH₂Cl₂ (3 Y 30 mL). The combined organic extract is dried over Na₂SO₄, evaporated and purified by flash chromatography (EtOAc:Hex, 1:9) to give 2-dimethylamino-9-fluorenone (220 mg, 62%). ¹H NMR (200 MHz, CDCl₃): δ 2.99 (s, 6H), 6.62 (dd, J=2.60 Hz, J=8.29 Hz, 1H), 7.01 (d, J=2.55 Hz, 1H), 7.08 (dt, J=1.46 Hz, J=7.18 Hz, 1H), 7.26-7.40 (m, 3H), 7.51-7.55 (m, 1H); ¹³C NMR (50 MHz, CDCl₃): δ40.52, 108.27, 116.42, 118.86, 121.10, 124.03, 126.73, 131.88, 134.17, 134.70, 135.65, 145.95, 151.25, 194.92; Anal. Calcd for C₁₅H₁₃NO-0.4H₂O: C, 78.17; H, 6.04. Found: C, 78.60; H, 6.00.

### EXAMPLE 26

### 3-Dimethylamino-9-fluorenone (4b-VIII)

The same reaction as described above for preparing 4a-VIII was employed, and 4b-VIII was obtained in 70% from 3-amino-9-fluorenone. ¹H NMR (200 MHz, CDCl₃): δ 3.00 (s, 6H), 6.31 (dd, J=2.31 Hz, J=8.51 Hz, 1H), 6.61 (d, J=2.28 Hz, 1H), 7.16-7.26 (m, 1H), 7.30-7.39 (m, 2H), 7.43-7.55 (m, 2H); ¹³C NMR (50 MHz, CDCl₃): δ40.16, 102.95, 110.06, 119.41, 121.74, 122.92, 126.09, 128.68, 133.02, 136.39, 143.36, 146.72, 154.84, 191.89; Anal. Calcd for C₁₅H₁₃NO-0.2H₂O: C, 79.41; H, 5.95. Found: C, 79.59; H, 5.63.

### EXAMPLE 27

### 4-Dimethylamino-9-fluorenone (4c-VIII)

The same reaction as described above for preparing 4a-VIII was employed, and 4c-VIII was obtained in 61% from 4-amino-9-fluorenone.

¹H NMR (200 MHz, CDCl₃): δ 2.75 (s, 6H), 7.14-7.21 (m, 3H), 7.29-7.33 (m, 1H), 7.39-7.42 (m, 1H), 7.59 (d, J=7.28 Hz, 1H), 7.77 (d, J=7.52 Hz, 1H); ¹³C NMR (50 MHz, CDCl₃): δ 43.92, 118.04, 123.51, 123.98, 125.06, 127.73, 129.65, 133.65, 134.46, 135.59, 135.78, 143.98, 149.93, 193.99; Anal. Calcd for C₁₅H₁₃NO: C, 80.69; H, 5.87. Found: C, 80.66; H, 5.79.

### EXAMPLE 28

### 2-Dimethylamino-7-bromo-9-fluorenone (4d-VIII)

The same reaction as described above for preparing 4a-VIII was employed, and 4d-VIII was obtained in 18% from 2-amino-7-bromo-9-fluorenone.

¹H NMR (200 MHz, CDCl₃): δ 3.02 (s, 6H), 6.70 (dd, J=2.59 Hz, J=8.32 Hz, 1H), 7.02 (d, J=2.54 Hz, 1H), 7.16-7.32 (m, 2H), 7.48 (dd, J=1.92 Hz, J=7.94 Hz, 1H), 7.64 (d, J=1.71 Hz, 1H); ¹³C NMR (50 MHz, CDCl₃): δ 40.55, 108.42, 116.64, 120.27, 121.35, 127.30, 131.03, 135.37, 135.90, 137.07, 144.66, 151.48, 193.52; HRMS: m/z Calcd for C₁₅H₁₂BrNO: 301.0102; Found: 301.0105; Anal. Calcd for C₁₅H₁₂BrNO: C, 59.62; H, 4.00. Found: C, 59.39; H, 3.80.

### EXAMPLE 29

### 2-Dimethylamino-7-(tributylstannyl)fluorene (5-VIII)

A mixture of 2-dimethylamino-7-bromofluorene (52 mg, 0.2 mmol), bis(tributylyltin) (0.2 mL) and Pd(Ph₃P)₄ (20 mg) in a mixed solvent (12 mL, dioxane:triethylamine, 3:1) was stirred at 90° C. overnight. Solvent was removed and the residue was purified by PTLC (Hex:EtOAc, 4:1) to give 23 mg of product, 5-VIII (yield 23%, not optimized). ¹H NMR (200 MHz, CDCl₃): δ 0.90 (t, J=7.17 Hz, 9H), 1.03-1.66 (m, 18H), 3.02 (s, 6H), 3.85 (s, 2H), 6.76 (dd, J=8.48 Hz, J=2.32 Hz, 1H), 6.94 (s, 1H), 7.37-7.64 (m, 4H); ¹³C NMR (50 MHz, CDCl₃): δ 9.67, 13.68, 27.41, 29.16, 37.06, 41.03, 109.28, 111.59, 118.20, 120.36, 131.32, 132.60, 134.54, 137.50, 141.97, 142.29, 144.83, 150.32; HRMS: m/z Calcd for C₂₇H₄₁NSn: 499.2261; Found: 499.2286.

### EXAMPLE 30

### Preparation of Radioiodinated Ligand: [¹²⁵I]2f-VIII

[¹²⁵I]TZDM was prepared according to the method described previously (23). The desired [¹²⁵I]2f-VIII was prepared using iododestannylation reactions with tributyltin precursors, 12-VIII. Hydrogen peroxide (50 μL, 3% w/v) was added to a mixture of 50 μL of the correspondent tributyltin precursor (1 μg/μL EtOH), 50 μL of 1N HCl and [¹²⁵I]NaI (1-5 mCi) in a sealed vial. The reaction was allowed to proceed for 10 min at room temperature and terminated by addition of 100 μL of sat. NaHSO₃. The reaction mixture was extracted with ethyl acetate (3×1 mL) after neutralization with saturated sodium bicarbonate solution. The combined extracts were evaporated to dryness. The residues were dissolved in 100 μL of EtOH and purified by HPLC using a reverse phase column (PRP-1, 4.6×250 mm) eluted with 100% acetonitrile—in a flow rate of 1.0 mL/min (retention time was around 12 to 13 minutes). The no-carrier-added product was evaporated to dryness and re-dissolved in 100% EtOH (1 μCi/μL). The final [¹²⁵I]2f-VIII, with a specific activity of 2,200 Ci/mmole and a greater than 95% radiochemical purity, was stored at −20° C. up to 6 weeks for autoradiography studies and animal distribution.

### EXAMPLE 31

### Dimethyl-(4′-amino-biphenyl-4-yl)-amine (2-VII)

The mixture of dimethyl-(4′-nitro-biphenyl-4-yl)-amine (1) (1 g, 4.1 mmol) and Pd/C (200 mg, 10% pd on carbon) in a mixed solvent (150 mL, EtOAc; EtOH=2:1) was hydrogenated at 55 psi for 4 h. The mixture was filtered and the filtrate was concentrated to give clean product 2-VII which was used as the starting material without further purification. ¹H NMR (200 MHZ, CDCL₃): 2.98 (S, 6H), 6.73 (D, T, J=8.5, 2.0 HZ, 2H), 6.80 (D, T, J=8.9, 2.0 HZ, 2H), 7.38 (D, T, J=8.5, 2.0 h, 2H), 7.44 D, T, J=8.9, 2.0 HZ, 2H). Anal. (C₁₄H₁₆N₂)

### EXAMPLE 32

### Dimethyl-(4′-N-methylamino-biphenyl-4-yl)-amine (3-VII)

To a mixture of 2-VII (100 mg, 0.47 mmol) in MeOH (10 mL) was added NaOMe solution (0.5 mL, 25% in MeOH) dropwise at RT followed by (CH₂O)ₙ (60 mg, 1.9 mmol). The resulting mixture was stirred under reflux for 2 h. NaBH₄ (50 mg 1.3 mmol) was added with caution after the reaction mixture was sooled down to RT. The mixture was refluxed for 1 h and cooled down. Water (10 mL) was added followed by NaOH solution (5 mL, 1M). The mixture was extracted with CH₂CL₂. Usual work up gave crude product which was purified by PTLC (Hex:EtOAc=3:1 as developing solvent) to give 84 mg of 3 (79%). ¹H NMR (200 MHz, CDCl₃): 2.89 (s, 3H), 3.00 (s, 6H), 6.69 (d, J=8.4 Hz, 2H), 6.83 (d, t, J=8.8, 2.0 Hz, 2H), 7.44 (d, t, J=8.4, 2.0 Hz, 2H), 7.49 (d, t, J=8.8, 2.0 Hz, 2H). Anal. (C₁₅H₁₈N₂)

### EXAMPLE 33

### Dimethyl-(4′-N-dimethylamino-biphenyl-4-yl)-amine (4-VII)

To a mixture of 2 (100 mg, 0.47 mmol) and (CH₂O)ₙ (200 mg, 6.3 mmol) in AcOH (5 mL) was added NaCNBH₃ (300 mg, 4.8 mmol) in one portion at RT. The mixture was stirred at RT overnight and poured into ice cold NaOH solution (15 mL, 25%). The resulting mixture was extracted with CH₂Cl₂. The organic phase was dried over Na₂SO₄, filtered, concentrated and purified by PTLC (Hex:EtOAc=3:1 as developing solvent) to give 93 mg of 4-VII (82%). ¹H NMR (200 MHz, CDCl₃): 2.99 (s, 12H), 6.82 (d, t, J=8.8, 2.0 Hz, 4H), 7.48 (d, t, J=8.8, 2.0 Hz, 4H). Anal. (C₁₆H₂₀N₂)

### EXAMPLE 34

### Dimethyl-(4′-hydroxy-biphenyl-4-yl)-amine (7-VII)

A mixture of boric acid 5-VII (165 mg, 1 mmol) and 4-iodophenol 6-VII (220 mg, 1 mmol), K2CO3 (276 mg, 2 mmol) and Pd(Ph3P)4 (28 mg, 0.024 mmol) in anhydrous MeOH (5 mL) was stirred at 60° C. overnight. The mixture was filtered and washed with CH₂Cl₂. The filtrate washed with water, dried, filtered, concentrated and purified by PTLC (Hex:EtOAc=3:1 as developing solvent) to give 125 mg of 7-VII (59%). ¹H NMR (200 MHz, CDCl₃): 2.98 (s, 6H), 6.80 (d, t, J=8.9, 2.0 Hz, 2H), 6.86 (d, t, J=8.7, 2.0 Hz, 2H), 7.43 (d, t, J=8.7, 2.0 Hz, 2H), 7.45 (d, t, J=8.9, 2.0 Hz, 2H). Anal. (C₁₄H₁₅NO)

### EXAMPLE 35

### Dimethyl-4-iodoaniline (9-VII)

Same procedure described above for preparation of 4-VII was performed to give product 9-VII in 62% yield starting from 4-iodoaniline 8-VII. ¹H NMR (200 MHz, CDCl₃): 2.92 (s, 6H), 6.49 (d, t, J=9.1, 2.0 Hz, 2H), 7.47 (d, t, J=9.1, 2.0 Hz, 2H). Anal. (C₈H₁₀IN)

### EXAMPLE 36

### Dimethyl-(3′-methoxycarbonyl-4′-amino-biphenyl-4-yl)-amine (11-VII)

Same procedure described above for preparation of 7-VII was performed to give product 11 in 57% yield starting from boric acid 5-VII and 10-VII. ¹H NMR (200 MHz, CDCl₃): 2.98 (s, 6H), 3.90 (s, 3H), 5.69 (br, 2H), 6.72 (d, J=8.5 Hz, 1H), 6.80 (d, J=8.8 Hz, 2H), 7.45 (d, J=8.8 Hz, 2H), 7.52 (d, d, J=8.5, 2.3 Hz, 1H), 8.08 (d, J=2.3 Hz, 1H).

### EXAMPLE 37

### Dimethyl-[3′-methoxycarbonyl-4′-(2″-p-methoxybenzylmercaptan)-acetylamino-biphenyl-4-yl]-amine (13-VII)

To a solution of acid 12-VII (509 mg, 2.4 mmol) in CH₂Cl₂ (5 mL) was added a solution of oxalyl chloride (2 mL, 2 M in CH₂Cl₂) dropwise at RT followed by DMF (3 drops). The mixture was stirred at RT for 1 h. Solvent was removed on the rotavapor. To the residue was added CH₂Cl₂ (5 mL) and cold to 0° C. in an ice bath. A solution of amine 11-VII (541 mg, 2.0 mmol) and Et₃N (0.7 mL, 5.0 mmol) in CH₂Cl₂ (10 mL) was added dropwise at 0° C. The resulting mixture was stirred at RT for 1 h. Water was added and the organic phase was dried, filtered, concentrated and purified by flash40 (Hex:EtOAc=4:1 as eluent) to give 600 mg of 13-VII (65%). ¹H NMR (200 MHz, CDCl₃): 3.00 (s, 6H), 3.32 (s, 2H), 3.71 (s, 3H), 3.80 (s, 2H), 3.99 (s, 3H), 6.79 (d, J=8.7 Hz, 2H), 6.81 (d, J=8.8 Hz, 2H), 7.25 (d, J=8.7 Hz, 2H), 7.51 (d, J=8.8 Hz, 2H), 7.74 (d, d, J=8.8, 2.3 Hz, 1H), 8.23 (d, J=2.3 Hz, 1H), 8.70 (d, J=8.8 Hz, 1H).

### EXAMPLE 38

### Dimethyl-[3′-hydroxycarbonyl-4′-(2″-p-methoxybenzylmercaptan)-acetylamino-biphenyl-4-yl]-amine (14-VII)

A mixture of compound 13-VII (240 mg, 0.52 mmol) and LiOH (120 mg, 5 mmol) in mixed solvent (10 mL, THF:MeOH:H₂O=3:1:1) was stirred at RT overnight. Solvent was removed under vacuum and neutralized with 10% HCl to PH 7. The mixture was extracted with mixed solvent (CH₂Cl₂:MeOH=9:1). The organic phase was dried, filtered, concentrated to give 230 mg of acid 14-VII (99%) which was pure enough to run the next reaction without further purification. ¹H NMR (200 MHz, CDCl₃): 3.01 (s, 6H), 3.34 (s, 2H), 3.65 (s, 3H), 3.78 (s, 2H), 6.76 (d, J=8.4 Hz, 2H), 6.84 (d, J=8.6 Hz, 2H), 7.22 (d, J=8.5 Hz, 2H), 7.52 (d, J=8.6 Hz, 2H), 7.80 (d, d, J=8.8, 2.1 Hz, 1H), 8.33 (d, J=2.1 Hz, 1H), 8.73 (d, J=8.8 Hz, 1H).

### EXAMPLE 39

### Dimethyl-[3′-(2″-p-methoxybenzylmercaptan)-ethylaminocarbonyl-4′-(2″-p-methoxybenzylmercaptan)-acetylamino-biphenyl-4-yl]-amine (16-VII)

To a mixture of acid 14-VII (230 mg, 0.51 mmol) and amine 15-VII (110 mg, 0.56 mmol) in CH₂Cl₂ (5 mL) was added DCC (105 mg, 0.51 mmol) in solid form followed by HOBT (69 mg, 0.51 mmol). The mixture was stirred at RT overnight. Solvent was removed after filtration and purified by flash40 (Hex:EtOAc=5:1 as eluent) to give 150 mg of 16-VII (47%). ¹H NMR (200 MHz, CDCl₃): 2.70 (t, J=6.4 Hz, 2H), 3.01 (s, 6H), 3.25 (s, 2H), 3.60 (q, J=6.2 Hz, 2H), 3.71 (s, 2H), 3.73 (s, 3H), 3.75 (s, 3H), 3.79 (s, 2H), 6.59 (t, J=5.4 Hz, 1H), 6.80 (d, J=8.4 Hz, 4H), 6.82 (d, J=8.6 Hz, 2H), 7.23 (d, J=8.5 Hz, 2H), 7.26 (d, J=8.5 Hz, 2H), 7.47 (d, J=8.7 Hz, 2H), 7.60 (d, J=1.9 Hz, 1H), 7.65 (d, d, J=8.5, 2.1 Hz, 1H), 8.55 (d, J=8.6 Hz, 1H), 11.42 (s, 1H).

### EXAMPLE 40

### Dimethyl-[3′-(2″-p-methoxybenzylmercaptan)-ethylaminomethyl-4′-(2″-p-methoxybenzylmercaptan)-ethylamino-biphenyl-4-yl]-amine (17-VII)

To a solution of 16-VII (100 mg, 0.16 mmol) in THF (10 mL) was added BH₃-THF (3 mL, 1 M in THF) dropwise at RT. The mixture was stirred under reflux overnight. Water was added carefully to destroy the excess BH₃. Solvent was removed and to the residue was added HCl (10 mL, 10%). The mixture was refluxed for 1 h. The cold mixture was made basic with concentrated NH₄OH and extracted with mixed solvent (CH₂Cl₂:MeOH=9:1). The organic phase was dried, filtered, concentrated and purified by PTLC (CH₂Cl₂:MeOH=97:3 as developing solvent) to give 41 mg of 17-VII (43%). ¹H NMR (200 MHz, CDCl₃): 2.61 (q, J=6.4 Hz, 2H), 2.74 (q, J=6.7 Hz, 2H), 2.77 (q, J=6.7 Hz, 2H), 2.98 (s, 6H), 3.33 (t, J=6.7 Hz, 2H), 3.65 (s, 2H), 3.69 (s, 2H), 3.78 (s, 3H), 3.79 (s, 3H), 3.80 (s, 2H), 6.60 (d, J=8.4 Hz, 1H), 6.75-6.88 (m, 6H), 7.19-7.26 (m, 4H), 7.37 (d, d, J=8.3, 2.1 Hz, 1H), 7.45 (d, J=8.7 Hz, 3H).

### EXAMPLE 41

### Dimethyl-[3′-(2″-mercaptan)-ethylaminomethyl-4′-(2″-mercaptan)-ethylamino-biphenyl-4-yl]-amine (18-VII)

To a solution of 17-VII (52 mg 0.09 mmol) in TFA (1.5 mL) was added anisole (3 drops) at RT. The mixture was cooled down to 0° C. in an ice bath. MeSO₃H (0.75 mL) was added dropwise at 0° C. The mixture was stirred at RT for 1 h. Ice water was added. The resulting mixture was extracted with ether (3 times). The aqueous phase was made basic with naHCO3 and extracted with mixed solvent (CH₂Cl₂:MeOH=9:1). The organic phase was dried, filtered, concentrated to give 26 mg of product 18-VII (84%). ¹H NMR (200 MHz, CDCl₃): 2.86-3.00 (m, 6H), 2.97 (s, 6H), 3.46-3.50 (m, 2H), 3.84 (s, 2H), 6.65-6.80 (m, 3H), 7.24 (s, 1H), 7.40-7.43 (m, 3H).

### EXAMPLE 42

### 4-amino-4-bromobiphenyl (20-VII)

To a mixture of 4-bromo-4-nitrobiphenyl 19-VII (48 mg, 0.17 mmol) in 3 ml of EtOH was poured SnCl₂ (54 mg, 0.34 mmol) in one portion. The resulting mixture was stirred at reflux for 1 h, then carefully poured into saturated NaHCO₃ to make neutral solution and extracted with methylene chloride. The combine extracts were dried, filtered, and concentrated in vacuo. The residue was subjected to flash chromatography to give 30 mg of 4-amino-4-bromobiphenyl 20-VII (71%). ¹H NMR (200 MHz, CDCl₃): *3.05 (s, 6H), 3.60 (br s, 2H), 6.74 (m, 2H), 7.40 (m, 4H), 7.51 (m, 2H); ¹³C NMR (50 MHz, CDCl₃): *115.37, 120.22, 127.80, 127.92, 130.20, 131.67, 140.07, 146.16.

### EXAMPLE 43

### 4-bromo-4-dimethylaminobiphenyl (21-VII)

To a stirred mixture of 4-amino-4-bromobiphenyl 20-VII (30 mg, 0.12 mmol) and papraformaldehyde (30 mg, 1 mmol) in 2 ml of AcOH at room temperature was added in one portion of NaCNBH₃. (31 mg, 0.5 mmol) The resulting mixture was stirred at room temperature for 18 h, then carefully poured into 25% aq. NaOH and ice chips to make strongly alkaline (pH 11) and extracted with methylene chloride. The combine extracts were dried, filtered, and concentrated in vacuo. The residue was subjected to flash chromatography. (EtOAc:Hex, 1:4) and gave 26 mg of 4-bromo-4-dimethylaminobiphenyl 21-VII (79%). ¹H NMR (200 MHz, CDCl₃): *3.00 (s, 6H), 6.79 (m, 2H), 7.39-7.53 (m, 6H); ¹³C NMR (50 MHz, CDCl₃): *40.45, 112.71, 119.88, 127.47, 127.76, 131.65, 140.12, 150.16.

### EXAMPLE 44

### 4-tributylstannous-4-dimethylbiphenyl (22-VII)

A mixture of 4-bromo-4-dimethylaminobiphenyl 21-VII (20 mg, 0.07 mmol), bis-(tributylyltin) (0.1 mL) and Pd(Ph₃P)₄ (10 mg) in a mixed solvent (4 mL, dioxane:triethylamine, 3:1) was stirred at 90° C. overnight. Solvent was removed and the residue was purified by PTLC (Hex:EtOAc, 4:1) to give 2.8 mg of product 22-VII (8%, not optimized yield). ¹H NMR (200 MHz, CDCl₃): *0.90 (t, J=7.17 Hz, 9H), 1.6 (t, J=8.31 Hz, 6H), 1.26-1.61 (m, 12H), 2.99 (s, 6H), 6.81 (m, 2H), 7.49-7.53 (m, 6H);

### EXAMPLE 45

### Iodide Derivatives: 4-iodo-4-dimethylaminobiphenyl

The same procedure as that of bromide.

¹H NMR (200 MHz, CDCl₃): *3.00 (s, 6H), 6.79 (m, 2H), 7.29 (m, 2H), 7.45 (m, 2H), 7.69 (m, 2H); ¹³C NMR (50 MHz, CDCl₃): *40.47, 91.09, 112.70, 127.45, 127.80, 128.10, 137.64, 140.72, 150.22.

### EXAMPLE 46

### Binding Assays of Several Compounds of Formula VIII Using Aggregated Aβ40 Peptide in Solution

The solid form of peptide Aβ40 was purchased from Bachem (King of Prussia, Pa.). Aggregation of peptide was carried out by gently dissolving the peptide (0.5 mg/mL) in a buffer solution (pH 7.4) containing 10 mM sodium phosphate and 1 mM EDTA. The solutions were incubated at 37° C. for 36-42 h with gentle and constant shaking. Binding studies were carried out in 12×75 mm borosilicate glass tubes according to the procedure described (8) with some modifications. For inhibition studies, 1 mL of the reaction mixture contained 40 if of inhibitors (concentration range between 10⁻⁵-10⁻¹⁰ M diluted in 10% EtOH), 50 μl of aggregated fibrils (10-50 nM in the final assay mixture) and 0.05 nM of radiotracer in 40% EtOH were used. The ethanol is needed for this assay, without which the some of the “cold” ligands evaluated were not soluble. Nonspecific binding was defined in the presence of 2 μM Thioflavin-T. The mixture was incubated at room temperature for 3 hr and the bound and the free radioactivity were separated by vacuum filtration through Whatman GF/B filters using a Brandel M-24R cell harvester followed by 2×3 mL washes of 10% ethanol at room temperature. Filters containing the bound I-125 ligand were counted in a gamma counter (Packard 5000) with 70% counting efficiency. The results of inhibition experiments were subjected to nonlinear regression analysis using software EBDA (15) by Kᵢ values were calculated.

Using in vitro binding assay it was demonstrated that substituted fluorenes competed with [¹²⁵I]TZDM binding to Aβ40 aggregates showing excellent binding affinities (Schemes 25, 26 and 27). When fluorenes with un-methylated amino groups, 1a-VIII-1f-VIII, were tested, only 3-aminofluorene, 1b-VIII, displayed a moderate binding affinity (Kᵢ=149 nM) (Scheme 25). However, the corresponding bromo-derivative, 1d-VIII, showed a higher binding affinity (Kᵢ=56±2 nM). When the aminofluorenes were transformed to the N,N-dimethylamino derivatives (2a-VIII-2f-VIII), they dramatically increased the binding affinities to Aβ aggregates. Especially, the 7-bromo- and 7-iodo-2-N,N-dimethylaminofluorene, 2d-VIII and 2f-VIII, displayed excellent binding affinities (Kᵢ=0.85±0.1 and 0.92±0.1 nM, respectively). It is also noted that 7-N,N-dimethylamino-2-N,N-dimethylaminofluorene, 2e-VIII, also showed a very good binding affinity (Kᵢ=15.4±5 nM). The 9-hydroxyfluorenes, 3a-VIII-3d-VIII, in general showed less potency in binding to Aβ aggregates. However, 7-bromo-2-N,N-dimethylamino-9-hydroxyfluorene, 3c-VIII, displayed a moderate potency (Kᵢ=88 nM). While the corresponding 7-bromo-2-N,N-dimethylaminofluorenone, 4d-VIII, was more potent (Kᵢ=16.5±4 mM). Based on the binding data it is reasonable to conclude that for this series of fluorene derivatives with a rigid tricyclic system, a 2- or 3-substituted N,N-dimethylamino group is needed to improve binding affinity.

### EXAMPLE 47

### In Vivo Biodistribution in Normal Mice of Several Compounds of Formula VIII

While under ether anesthesia, 0.15 mL of a 0.1% bovine serum albumin solution containing [¹²⁵I]2f-VIII (5-10 μCi) was injected directly into the tail vein of male ICR mice (2-3 month-old, average weight 20-30 g). The mice were sacrificed by cardiac excision at various time points post injection. The organs of interest were removed and weighed, and the radioactivity was counted with an automatic gamma counter (Packard 5000). The percentage dose per organ was calculated by a comparison of the tissue counts to suitably diluted aliquots of the injected material. Total activities of blood and muscle were calculated under the assumption that they were 7% and 40% of the total body weight, respectively.

Biodistribution studies in a group of normal mice after an iv injection showed that [¹²⁵I]2f-VIII exhibited an excellent brain uptake (1.13% ID/organ at 2 min) and peaked at 1 h (1.26% ID/organ) (Table 1). At 1 and 6 h there was 0.72 and 0.17% ID/organ, respectively remained in the brain. The blood levels are relatively low at all time point measured (4-6% ED/organ). The tracer seems to distribute in high blood flow areas, such as liver, kidney, muscle and skin (Table 1). The partition coefficient (P.C) of [¹²⁵I]2f-VIII is 294 (1-octanol/buffer), which is comparable to that of TZDM (P.C.=70) (23). A relatively good lipophilicity is essential for the initial brain penetration by a simple diffusion mechanism.

TABLE 1

| | | | | |
|---|---|---|---|---|
| Organ | 2 min | 30 min | 60 min | 240 min |

| | | | | | | | | |
|---|---|---|---|---|---|---|---|---|
| Blood | 6.50 | ±0.75 | 4.88 | ±0.90 | 4.81 | ±0.88 | 4.09 | ±0.25 |
| Heart | 1.30 | ±0.15 | 0.21 | ±0.03 | 0.19 | ±0.02 | 0.11 | ±0.02 |
| Muscle | 11.35 | ±1.43 | 9.24 | ±0.45 | 7.46 | ±1.49 | 6.21 | ±0.17 |
| Lung | 2.46 | ±0.81 | 0.76 | ±0.15 | 0.48 | ±0.06 | 0.33 | ±0.04 |
| Kidney | 4.96 | ±0.84 | 1.87 | ±0.22 | 1.46 | ±0.11 | 0.88 | ±0.10 |
| Spleen | 0.61 | ±0.08 | 0.25 | ±0.02 | 0.17 | ±0.03 | 0.18 | ±0.02 |
| Liver | 24.96 | ±2.45 | 8.84 | ±1.35 | 5.84 | ±0.88 | 5.71 | ±0.89 |
| Skin | 1.97 | ±0.68 | 7.40 | ±1.15 | 8.22 | ±0.30 | 5.66 | ±0.50 |
| Brain | 1.13 | ±0.06 | 1.26 | ±0.32 | 0.72 | ±0.03 | 0.17 | ±0.03 |

Biodistribution in mice after an intravenous injection of [¹²⁵I]2f-VIII % dose/organ, avg of 3 mice ± SD

The following Examples are directed to the synthesis of several compounds of Formula III.

### EXAMPLE 48

### 2-(4′-Dimethylaminophenyl)-6-iodobenzothiazole, (3-III)

2-(4′-Dimethylaminophenyl)-6-bromobenzothiazole (1-III): (Stevens, M. F. G., et al., J. Med. Chem. 37:1689-1695 (1994); Stevens, M. F. G. et al., PCT Int. Appl. WO19940830:47 (1995)). A mixture of 5-bromo-2-amino-benzenethiol (Mital, R. L. and Jain, S. K., J. Chem Soc (C):2148 (1969); Lin, A.-J. and Kasina, S., J Heterocycl Chem 18:759 (1981)) (306 mg, 1.5 mmol) and 4-dimethylamino benzaldehyde (224 mg, 1.5 mmol) in DMSO was heated at 180<C for 15 min. Water (10 mL) was added after the mixture was cooled down. The solid was collected by suction and recrystallized in ethyl acetate to give 340 mg of product (68%). ¹H NMR (200 MHz, CDCl₃): δ 3.06 (s, 6H), 6.74 (d, J=9.0 Hz, 2H), 7.52 (d, d, J=8.7, 2.0 Hz, 1H), 7.82 (d, J=8.6 Hz, 1H), 7.93 (d, J=8.8 Hz, 2H), 7.95 (s, 1H). HRMS: m/z Calcd for C₁₅H₁₄BrN₂S(MH⁺): 333.0061; Found: 333.0072.

### EXAMPLE 49

### 2-(4′-Dimethylaminophenyl)-6-tribytylstannylbenzothiazole (2-III)

To a solution of 2-(4′-dimethylaminophenyl)-6-bromobenzothiazole (16a-III) (60 mg, 0.18 mmol) in 1,4-dioxane (2 mL), toluene (2 mL) and triethylamine (2 mL) was added (Bu₃Sn)₂ (0.2 mL) followed by Pd(Ph₃P)₄ (20 mg). The mixture was stirred at 90° C. overnight. Solvent was removed and the residue was purified by PTLC (Hex:EtOAc, 6:1) to give 33 mg of product (yield 33.6%). ¹H NMR (200 MHz, CDCl₃): δ<0.90 (t, J=7.1 Hz, 9H), 1.10 (t, J=8.0 Hz, 6H), 1.34 (hex, J=7.3 Hz, 6H), 1.57 (m, 6H), 3.05 (s, 6H), 6.74 (d, J=9.0 Hz, 2H), 7.50 (d, d, J=7.9, 0.9 Hz, 1H), 7.93 (s, 1H), 7.95 (d, J=8.5 Hz, 1H), 7.97 (d, J=9.0 Hz, 2H). HRMS: m/z Calcd for C27H41N2SSn(MH+): 545.2012; Found: 545.2035.

### EXAMPLE 50

### 2-(4′-Dimethylaminophenyl)-6-iodobenzothiazole (3-III)

To a solution of 2-III (45 mg, 0.08 mmol) in CHCl₃ (10 mL) was added a solution of iodine (1 mL, 1M in CHCl₃) dropwise at RT until the color maintaining unchanged. The resulting mixture was stirred at RT for 10 min. NaHSO₃ solution (2 mL, 5% in water) and KF (1 mL, 1M in MeOH) were added successively. The mixture was stirred for 5 min an the organic phase was separated. The aqueous phase was extracted with CH2Cl2 and the combined organic phases was dried over Na2SO4, filtered and concentrated to give crude product which was purified by PTLC (Hex:EtOAc, 6:1) to give 9 mg of the desired product (yield 29%). ¹H NMR (200 MHz, CDCl₃): δ<3.06 (s, 6H), 6.73 (d, J=9.0 Hz, 2H), 7.69 (s, 1H), 7.70 (s, 1H), 7.93 (d, J=9.0 Hz, 2H), 8.15 (s, 1H). HRMS: m/z Calcd for C15H15N2IS(MH+): 380.9922; Found: 380.9914. Anal. (C15H14N31S): C, H, N.

### EXAMPLE 51

### 2-[4′-(4″-Methylpiperazin-1-yl)-phenyl]-6-iodobenzothiazole, (6-III)

2-[4′-(4″-Methylpiperazin-1-yl)-phenyl]-6-bromobenzothiazole (4-III): The procedure described above to prepare 1-III was employed to give 57.2% of product 4-III from 4-(4-methylpiperazin-1-yl)benzaldehyde (Tanaka, A., et al., J. Med. Chem. 41:2390 (1998)) (204 mg, 1 mmol) and 5-bromo-2-amino-benzenethiol (204 mg, 1 mmol). ¹H NMR (200 MHz, CDCl₃): δ<2.38 (s, 3H), 2.60 (t, J=5.0 Hz, 4H), 3.38 (t, J=5.0 Hz, 4H), 6.96 (d, J=8.9 Hz, 2H), 7.54 (d, d, J=8.5, 1.9 Hz, 1H), 7.83 (d, J=8.5 Hz, 1H), 7.95 (d, J=8.9 Hz, 2H), 7.98 (s, 1H). HRMS: m/z Calcd for C₁₈H₁₉BrN₃S(MH⁺): 388.0483; Found: 388.0474.

2-[4′-(4″-Methylpiperazin-1-yl)-phenyl]-6-tributylstannyl benzothiazole (5-III): The procedure described above to prepare 2-III was employed, 5-III was obtained in 23% yield from 4-III. ¹H NMR (200 MHz, CDCl₃): δ 0.89 (t, J=7.2 Hz, 9H), 1.06 (t, J=8.2 Hz, 6H), 1.30 (hex, J=7.3 Hz, 6H), 1.57 (pen, J=7.2 Hz, 6H), 2.38 (s, 3H), 2.60 (m, 4H), 3.36 (t, J=5.0 Hz, 4H), 6.96 (d, J=8.9 Hz, 2H), 7.52 (d, J=7.9 Hz, 1H), 7.93 (s, 1H), 7.95 (d, J=7.9 Hz, 1H), 7.98 (d, J=8.9 Hz, 2H). HRMS: m/z Calcd for C30H46N3SSn(MH+): 600.2434; Found: 600.2449.

2-[4′-(4″-Methylpiperazin-1-yl)-phenyl]-6-iodobenzothiazole, (6-III): The same reaction as described above to prepare 3-III was employed, 6-III was obtained in 36% yield from 5-III. ¹H NMR (200 MHz, CDCl₃): δ 2.42 (s, 3H), 263 (t, J=4.8 Hz, 4H), 3.40 (t, J=4.9 Hz, 4H), 6.95 (d, J=9.0 Hz, 2H), 7.71 (s, 1H), 7.72 (s, 1H), 7.95 (d, J=8.9 Hz, 2H), 8.17 (t, J=1.0 Hz, 1H) HRMS: m/z Calcd for C₁₈H₁₉N₃IS(MH⁺): 436.0344; Found: 436.0364. Anal. (C₁₈H₁₈N₃SI): C, H, N.

The following Examples are directed to the synthesis of several compounds of Formula V.

### EXAMPLE 52

### Preparation of 6-Tributylstannyl-2-(4′-dimethylamino-)phenyl-imidazo[1,2a]pyridine (18-V)

6-Bromo-2-(4′-dimethylamino-)phenyl-imidazo[1,2-a]pyridine (17-V) A mixture of 2-bromo-4′-dimethylaminoacetophenone, (968 mg, 4 mmol) and 2-amino-5-bromo-pyridine (692 mg, 4 mmol) in EtOH (25 mL) was stirred under reflux for 2 hr. NaHCO₃ (500 mg) was added after the mixture was cooled down. The resulting mixture was stirred under reflux for 4.5 hr. The mixture was cooled down, filtered to give 655 mg of product, 17 (52%). ¹H NMR (200 MHz, CDCl₃, δ): 3.00 (s, 6H), 6.78 (d, J=8.7 Hz, 2H), 7.17 (d, d, J=9.5, 1.7 Hz, 1H), 7.49 (d, J=9.5 Hz, 1H), 7.69 (s, 1H), 7.80 (d, J=8.7 Hz, 2H), 8.21 (d, d, J=1.7, 0.8 Hz, 1H). Anal 3a, (C₁₅H₁₄BrN₃).

6-Tributylstannyl-2-(4′-dimethylamino-)phenyl-imidazo[1,2-a]pyridine (18-V). To a solution of 6-bromo-2-(4′-dimethylamino-)phenyl-imidazo[1,2-a]pyridine, 17-V, (80 mg, 0.26 mmol) in 1,4-dioxane (10 mL) and triethylamine (2 mL) was added (Bu₃Sn)₂ (0.2 mL) in neat followed by Pd(Ph₃P)₄ (20 mg). The mixture was stirred at 90° C. overnight. Solvent was removed and the residue was purified by PTLC (Hex:EtOAc=1:1 as developing solvent) to give 23 mg of product, 18-V (17%). ¹H NMR (200 MHz, CDCl₃, δ): 0.90 (t, J=7.2 Hz, 9H), 1.10 (t, J=8.0 Hz, 6H), 1.33 (hex, J=7.1 Hz, 6H), 1.54 (pen, J=7.2 Hz, 6H), 3.00 (s, 6H), 6.78 (d, J=8.9 Hz, 2H), 7.11 (d, J=8.8 Hz, 1H), 7.57 (d, J=8.8 Hz, 1H), 7.71 (s, 1H), 7.84 (d, J=8.8 Hz, 2H), 7.95 (d, J=0.8 Hz, 1H). HRMS: m/z Calcld for C₂₇H₄₂N₃Sn(M⁺⁺H): 528.2400; Found: 528.2402. Anal. 4, (C₂₇H₄₁N₃Sn.2H₂O).

6-Iodo-2-(4′ dimethylamino-)phenyl-imidazo[1,2-a]pyridine, IMPY. A mixture of 2-bromo-4′-dimethylaminoacetophenone, (484 mg, 2 mmol) and 2-amino-5-iodo-pyridine (440 mg, 2 mmol) in EtOH (25 mL) was stirred under reflux for 2 hr. NaHCO₃ (250 mg) was added after the mixture was cooled down. The resulting mixture was stirred under reflux for 4 hr. The mixture was cooled down, filtered to give 348 mg of product in a yield of (48%). ¹H NMR (200 MHz, CDCl₃, δ): 3.00 (s, 6H), 6.77 (d, J=8.8 Hz, 2H), 7.27 (d, d, J=9.4, 1.5 Hz, 1H), 7.38 (d, J=9.5 Hz, 1H), 7.66 (s, 1H), 7.79 (d, J=8.8 Hz, 2H), 8.32 (d, J=0.7 Hz, 1H). Anal. 3b, (C₁₅H₁₄IN₃).

### EXAMPLE 53

### Preparation of Radioiodinated Ligand: [¹²⁵I]IMPY, [¹²⁵I]19-V

The compound, [¹²⁵I]19-V, was prepared using iododestannylation reactions with tributyltin precursor 18-V. Hydrogen peroxide (50 μL, 3% w/v) was added to a mixture of 50 μL of the correspondent tributyltin precursor (1 μg/μL EtOH), 50 μL of 1N HCl and [^{125/123}I]NaI (1-5 mCi) in a sealed vial. The reaction was allowed to proceed for 10 min at room temperature and terminated by addition of 100 μL of sat. NaHSO₃. The reaction mixture was either directly extracted (styrylbenzenes) with ethylacetate (3×1 mL) or extracted after neutralization with saturated sodium bicarbonate solution (thioflavins). The combined extracts were evaporated to dryness. For styrylbenzenes the residues were dissolved in 100 μL of EtOH and purified by HPLC using a reverse phase column (Waters ubondpad, 3.9×300 mm) with an isocratic solvent of 65% acetonitrile-35% trifluoroacetic acid (0.1%) in a flow rate of 0.8 mL/min. Thioflavins were purified on a C4 column (Phenomenex Inc., Torrance, Calif.) eluted with an isocratic solvent of 80% acetonitrile-20% 3,3-dimethyl-glutaric acids (5 mM, pH 7.0) in a flow rate of 0.8 mL/min. The desired fractions containing the product were collected, condensed and re-extracted with ethylacetate. The no-carrier-added products were evaporated to dryness and re-dissolved in 100% EtOH (1 μCi/μL). The final ¹²⁵I 19-V, with a specific activity of 2,200 Ci/mmole and a greater than 95% radiochemical purity, were stored at −20<C up to 6 weeks for in vitro binding and autoradiography studies.

### EXAMPLE 54

### Partition Coefficient Determination

Partition coefficients were measured by mixing the [¹²⁵I]tracer with 3 g each of 1-octanol and buffer (0.1 M phosphate, pH 7.4) in a test tube. The test tube was vortexed for 3 min at room temperature, followed by centrifugation for 5 min. Two weighed samples (0.5 g each) from the 1-octanol and buffer layers were counted in a well counter. The partition coefficient was determined by calculating the ratio of cpm/g of 1-octanol to that of buffer. Samples from the 1-octanol layer were re-partitioned until consistent partitions of coefficient values were obtained. The measurement was done in triplicate and repeated three times.

### EXAMPLE 55

### Binding Assays Using Aggregated Aβ(1-40) or Aβ(1-42) Peptide in Solution

The solid forms of peptides Aβ(1-40) and Aβ(1-42) were purchased from Bachem (King of Prussia, Pa.). Aggregation of peptides were carried out by gently dissolving the peptide [0.5 mg/mL for Aβ(1-40) and 0.25 mg/mL for Aβ (1-42) in a buffer solution (pH 7.4) containing 10 mM sodium phosphate and 1 mM EDTA. The solutions were incubated at 37° C. for 36-42 h with gentle and constant shaking. Binding studies were carried out in 12×75 mm borosilicate glass tubes according to the procedure described with some modifications (Klunk, W. E., et al., Biol. Psychiatry 35:627 (1994)). Aggregated fibrils (10-50 nM in the final assay mixture) were added to the mixture containing 50 ml of radioligands (0.01-0.5 nM) in 40% EtOH and 10% EtOH in a final volume of 1 mL for saturation studies. Nonspecific binding was defined in the presence of 2 mM thioflavin T for thioflavins. For inhibition studies, 1 mL of the reaction mixture contained 40 ml of inhibitors (10−5-10−10 M in 10% EtOH) and 0.05 nM radiotracer in 40% EtOH. The mixture was incubated at room temperature for 3 h and the bound and the free radioactivity were separated by vacuum filtration through Whatman GF/B filters using a Brandel M-24R cell harvester followed by 2×3 mL washes of 10% ethanol at room temperature. Filters containing the bound I-125 ligand were counted in a gamma counter (Packard 5000) with 70% counting efficiency. The results of saturation and inhibition experiments were subjected to nonlinear regression analysis using software EBDA52 by which Kd and Ki values were calculated. Results of the binding experiments are shown in Table 2.

TABLE 2
Inhibition constants (Ki, nM) of compounds on ligand binding
to aggregates of Aβ (1-40) and Aβ (1-42) at 25° C.

| | Aggregates of Aβ (1-40) | Aggregates of Aβ (1-42) |
|---|---|---|
| Compounds | vs[¹²⁵I]3 | vs[¹²⁵I]3 |
| Chrysamine G | >1,000 | >2,000 |
| Thioflavin T | 116 ± 20 | 294 ± 40 |
| 1 | 1.9 ± 0.3 | 0.8 ± 0.3 |
| 4 | 1.6 ± 0.5 | 5.0 ± 0.8 |
| 3 | 0.9 ± 0.2 | 2.2 ± 0.4 |
| 6a | 5.4 ± 0.7 | 6.4 ± 0.7 |

Values are the mean ±SEM of three independent experiments, each in duplicates.

### EXAMPLE 56

### In Vivo Biodistribution of New Probes in Normal Mice

While under ether anesthesia, 0.15 mL of a saline solution containing labeled agents (5-10 mCi) was injected directly into the tail vein of ICR mice (2-3 month-old, average weight 20-30 g). The mice were sacrificed by cardiac excision at various time points post injection. The organs of interest were removed and weighed, and the radioactivity was counted with an automatic gamma counter (Packard 5000). The percentage dose per organ was calculated by a comparison of the tissue counts to suitably diluted aliquots of the injected material. Total activities of blood and muscle were calculated under the assumption that they were 7% and 40% of the total body weight, respectively. The data are shown in Table 2.

TABLE 2

| | | | | | |
|---|---|---|---|---|---|
| Organ | 2 min | 30 min | 60 min | 6 h | 24 h |

[¹²⁵I] Compound 3 (PC = 70)

| | | | | | | | | | | |
|---|---|---|---|---|---|---|---|---|---|---|
| Blood | 15.74 | ±6.06 | 3.26 | ±0.05 | 3.79 | ±0.19 | 1.44 | ±0.05 | 0.29 | ±0.09 |
| Heart | 1.79 | ±0.39 | 0.20 | ±0.01 | 0.17 | ±0.02 | 0.05 | ±0.01 | 0.01 | ±0.00 |
| Liver | 31.62 | ±2.38 | 10.93 | ±2.34 | 9.21 | ±3.05 | 1.52 | ±0.30 | 0.30 | ±0.07 |
| Brain | 0.67 | ±0.11 | 0.97 | ±0.29 | 1.57 | ±0.24 | 0.65 | ±0.11 | 0.04 | ±0.01 |

[¹²⁵I] Compound 6a (PC = 312)

| | | | | | | | | | | |
|---|---|---|---|---|---|---|---|---|---|---|
| Blood | 8.02 | ±0.82 | 5.15 | ±0.23 | 4.16 | ±0.28 | 1.49 | ±0.26 | 0.41 | ±0.09 |
| Heart | 2.19 | ±0.43 | 0.69 | ±0.02 | 0.66 | ±0.06 | 0.22 | ±0.06 | 0.08 | ±0.01 |
| Liver | 28.84 | ±3.77 | 21.22 | ±5.86 | 17.20 | ±2.49 | 5.79 | ±1.24 | 3.05 | ±0.87 |
| Brain | 1.50 | ±0.10 | 1.59 | ±0.19 | 1.89 | ±0.43 | 1.08 | ±0.08 | 0.91 | ±0.08 |

[¹²⁵I] Compound 8 (PC = 124)

| | | | | | | | | | | |
|---|---|---|---|---|---|---|---|---|---|---|
| Blood | 4.31 | ±0.34 | 2.80 | ±0.45 | 2.94 | ±0.18 | 2.23 | ±0.53 | 1.68 | ±0.56 |
| Heart | 1.20 | ±0.18 | 0.19 | ±0.05 | 0.11 | ±0.02 | 0.05 | ±0.00 | 0.02 | ±0.00 |
| Liver | 25.04 | ±2.45 | 17.45 | ±2.01 | 5.57 | ±0.39 | 1.08 | ±0.11 | 0.42 | ±0.08 |
| Brain | 1.43 | ±0.23 | 2.08 | ±0.03 | 1.26 | ±0.10 | 0.12 | ±0.02 | 0.01 | ±0.00 |

| | | | | | | |
|---|---|---|---|---|---|---|
| Organ | 2 min | 30 min | 1 hr | 2 hr | 6 hr | 24 hr> |

[¹²⁵I] Compound 19 (PC = 100)

| | | | | | | |
|---|---|---|---|---|---|---|
| BLOOD | 6.41 ± 0.77 | 2.44 ± 0.36 | 2.50 ± 0.11 | 1.82 ± 0.21 | 1.40 ± 0.27 | 0.18 ± 0.02 |
| HEART | 0.79 ± 0.14 | 0.16 ± 0.02 | 0.12 ± 0.02 | 0.08 ± 0.01 | 0.04 ± 0.01 | 0.01 ± 0.00 |
| MUSCLE | 13.81 ± 3.44 | 6.08 ± 0.59 | 5.03 ± 1.03 | 2.96 ± 0.84 | 1.46 ± 0.42 | 0.27 ± 0.11 |
| LUNG | 1.56 ± 0.33 | 0.31 ± 0.07 | 0.34 ± 0.08 | 0.20 ± 0.05 | 0.12 ± 0.05 | 0.05 ± 0.03 |
| KIDNEY | 4.75 ± 0.49 | 1.51 ± 0.27 | 1.17 ± 0.29 | 0.53 ± 0.05 | 0.25 ± 0.05 | 0.05 ± 0.01 |
| SPLEEN | 0.40 ± 0.06 | 0.09 ± 0.02 | 0.08 ± 0.01 | 0.05 ± 0.01 | 0.04 ± 0.01 | 0.01 ± 0.00 |
| LIVER | 20.88 ± 2.63 | 6.32 ± 0.55 | 5.88 ± 0.85 | 2.90 ± 0.21 | 1.54 ± 0.08 | 0.61 ± 0.11 |
| SKIN | 5.72 ± 0.90 | 4.69 ± 1.06 | 4.28 ± 0.25 | 3.14 ± 0.51 | 2.19 ± 0.63 | 0.22 ± 0.06 |
| BRAIN | 2.88 ± 0.25 | 0.26 ± 0.00 | 0.21 ± 0.03 | 0.14 ± 0.03 | 0.06 ± 0.02 | 0.02 ± 0.00 |

% dose/organ, average of 3 mice ± SD; Average organ weights are: blood, 2 g; muscle, 12 g; liver, g; brain 0.4 g, from which the % dose/g value for each organ or tissue can be calculated.
*(% dose/organ, avg of 3 or 4 mice ± SD)

Having now fully described this invention, it will be understood to those of ordinary skill in the art that the same can be performed within a wide and equivalent range of conditions, formulations, and other parameters without affecting the scope of the invention or any embodiment thereof. All patents, patent applications, and publications cited herein are fully incorporated by reference herein in their entirety.

What is claimed is:

1. A pharmaceutical composition, comprising a compound having one of the following Formulae: 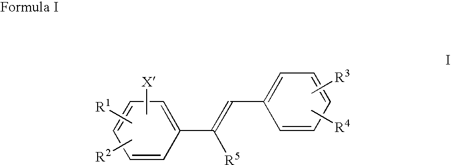
or a pharmaceutically acceptable salt thereof, wherein:
R⁵ is hydrogen or C₁₋₄ alkyl;
R¹, R² and R³, in each instance, is independently selected from the group consisting of hydrogen, hydroxy, halogen, C₁₋₄ alkyl, C₁₋₄ alkoxy, cyano, carboxy(C₁₋₅)alkyl, trifluoromethyl, nitro, methylamino, dimethylamino, halo(C₁₋₄)alkyl, and formyl;
R⁴ is selected from the group consisting of:
a. C₁₋₄ alkylthio,
b. C₁₋₄ alkylsulfonyl,
c. hydroxy,
d. C₁₋₄ alkoxy,
e. NR⁶R⁷, wherein
R⁶ and R⁷ are independently hydrogen or C₁₋₄ alkyl,
f. phenyl(C₁₋₄)alkyl,
g. C₆₋₁₀ aryl,
h. heteroaryl,
i. heterocycle,
j. heterocycle(C₁₋₄)alkyl, and
k. C₃₋₆ cycloalkyl,
wherein said phenyl(C₁₋₄)alkyl, C₆₋₁₀ aryl, heteroaryl, heterocycle, heterocycle(C₁₋₄)alkyl or C₃₋₆ cycloalkyl is substituted with one of the following: C₁₋₄ alkylthio, C₁₋₄ alkyl sulfonyl, methoxy, hydroxy, dimethylamino or methylamino;
or R³ and R⁴ are taken together to form an optionally substituted aryl or heteroaryl ring, wherein said ring is attached at adjacent carbons on the appropriate stilbene ring;
and,
X′ is selected from the group consisting of hydrogen, halogen, halo(C₁₋₄)alkyl, halo(C₁₋₄)alkyl amino, halo(C₁₋₄)alkyl(C₁₋₄ alkyl)amino, and Sn(alkyl)₃;
provided that,
in each instance, said halogen is other than a radiolabeled halogen; and
if R⁴ is other than NR⁶R⁷, then R¹ is methylamino or dimethyl amino; 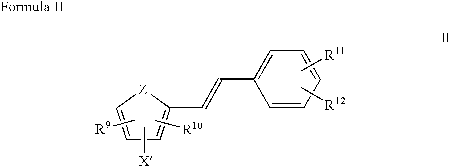
or a pharmaceutically acceptable salt thereof,
Z is O, S or NR^{a}, wherein
R^{a} is C₁₋₄ alkyl;
R⁹, R¹⁰ and R¹¹, in each instance, is independently selected from the group consisting of hydrogen, halogen, C₁₋₄ alkyl, cyano, carboxy(C₁₋₅)alkyl, trifluoromethyl, nitro, methylamino, dimethylamino, halo(C₁₋₄)alkyl, and formyl;
R¹² is selected from the group consisting of:
a. C₁₋₄ alkylthio,
b. C₁₋₄ alkylsulfonyl,
c. hydroxy,
d. C₁₋₄ alkoxy,
e. NR¹³R¹⁴, wherein
R¹³ and R¹⁴ are hydrogen or C₁₋₄ alkyl,
f. phenyl(C₁₋₄)alkyl,
g. C₆₋₁₀ aryl,
h. heteroaryl,
i. heterocycle,
j. heterocycle(C₁₋₄)alkyl, and
k. C₃₋₆ cycloalkyl,
wherein said phenyl(C₁₋₄)alkyl, C₆₋₁₀ aryl, heteroaryl, heterocycle, heterocycle(C₁₋₄)alkyl or C₃₋₆ cycloalkyl is substituted with one of the following: C₁₋₄ alkylthio, C₁₋₄ alkyl sulfonyl, methoxy, hydroxy, dimethylamino or methylamino;
or R¹¹ and R¹² are taken together to form an optionally substituted aryl or heteroaryl ring, wherein said ring is attached at adjacent carbons on the appropriate stilbene ring; and,
X′ is selected from the group consisting of hydrogen, halogen, halo(C₁₋₄)alkyl, halo(C₁₋₄)alkyl amino, halo(C₁₋₄)alkyl(C₁₋₄ alkyl)amino, and Sn(alkyl)₃;
provided that
in each instance, said halogen is other than a radiolabeled halogen; and
if R⁴ is other than NR⁶R⁷, then R¹ is methylamino or dimethyl amino; 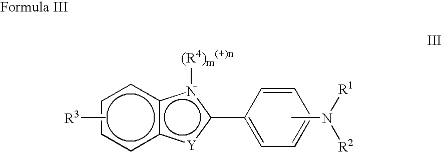
or a pharmaceutically acceptable salt thereof,
wherein:
Y is CH, NR⁵, O, S or CH═N, where R⁵ is hydrogen or a C₁₋₄ alkyl;
m and n are both zero, or m and n are both 1;
R³ is selected from the group consisting of —CH₃, hydrogen, halogen, halo(C₁₋₄)alkyl, halo(C₁₋₄)alkyl amino, halo(C₁₋₄)alkyl(C₁₋₄ alkyl)amino, and Sn(alkyl)₃;
R¹ and R² are independently hydrogen, C₁₋₄ alkyl, C₂₋₄ aminoalkyl, C₁₋₄ haloalkyl, haloarylalkyl, or R¹ and R² are taken together with the nitrogen to which they are attached to form a 5- to 7-member heterocyclic ring optionally having O, S or NR⁶ in said ring, where
R⁶ is hydrogen or C₁₋₄ alkyl; and
R⁴ is C₁₋₄ alkyl;
provided that in each instance, said halogen is other than a radiolabeled halogen; 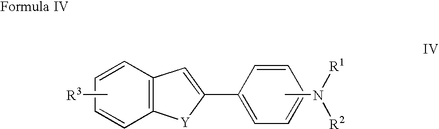
or a pharmaceutically acceptable salt thereof, wherein:
Y is O or NR⁴ where R⁴ is hydrogen or C₁₋₄ alkyl;
R³ is selected from the group consisting of hydrogen, halogen, halo(C₁₋₄)alkyl, halo(C₁₋₄)alkyl amino, halo(C₁₋₄)alkyl(C₁₋₄ alkyl)amino, and Sn(alkyl)₃;
R¹ and R² are independently hydrogen, C₁₋₄ alkyl, C₂₋₄ aminoalkyl, C₁₋₄ haloalkyl, haloarylalkyl, or R¹ and R² are taken together with the nitrogen to which they are attached to form a 5- to 7-member heterocyclic ring optionally having O, S or NR⁵ in said ring, where
R⁵ is hydrogen or C₁₋₄ alkyl;
provided that in each instance, said halogen is other than a radiolabeled halogen; 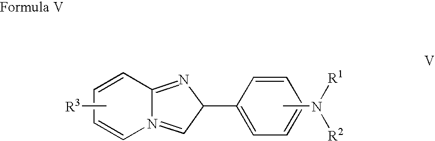
or a pharmaceutically acceptable salt thereof,
wherein:
R³ is selected from the group consisting of hydrogen, halogen, halo(C₁₋₄)alkyl, halo(C₁₋₄)alkyl amino, halo(C₁₋₄)alkyl(C₁₋₄ alkyl)amino, and Sn(alkyl)₃;
R¹ and R² are independently hydrogen, C₁₋₄ alkyl, C₂₋₄ aminoalkyl, C₁₋₄ haloalkyl, haloarylalkyl, or R¹ and R² are taken together with the nitrogen to which they are attached to form a 5- to 7-member heterocyclic ring optionally having O, S or NR⁵ in said ring, where
R⁵ is hydrogen or C₁₋₄ alkyl;
provided that in each instance, said halogen is other than a radiolabeled halogen; 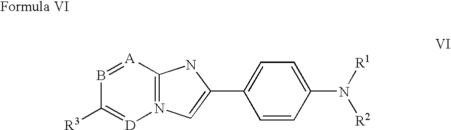
or a pharmaceutically acceptable salt thereof,
wherein:
A, B and D are CH or N,
provided that at least one, no more than two of A, B and D is N;
R³ is selected from the group consisting of hydrogen, halogen, halo(C₁₋₄)alkyl, halo(C₁₋₄)alkyl amino, halo(C₁₋₄)alkyl(C₁₋₄ alkyl)amino, and Sn(alkyl)₃;
R¹ and R² are independently hydrogen, C₁₋₄ alkyl, C₂₋₄ aminoalkyl, C₁₋₄ haloalkyl, haloarylalkyl, or R¹ and R² are taken together with the nitrogen to which they are attached to form a 5- to 7-member heterocyclic ring optionally having O, S or NR⁵ in said ring, where
R⁵ is hydrogen or C₁₋₄ alkyl;
provided that in each instance, said halogen is other than a radiolabeled halogen; 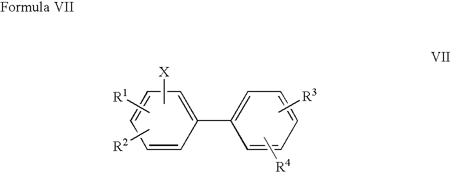
or a pharmaceutically acceptable salt thereof, wherein
R¹, R² and R³ are independently selected from the group consisting of hydrogen, halogen, C₁₋₅ alkyl, cyano, carboxy(C₁₋₅)alkyl, trifluoromethyl, nitro, methylamino, dimethylamino, halo(C₁₋₅)alkyl, hydroxy(C₁₋₅)alkyl, (Bu)₃Sn—, (Bu)₃Sn(C₁₋₅)alkyl and formyl,
R⁴ is selected from the group consisting of:
a. C₁₋₅ alkylthio,
b. halo(C₁₋₅)alkyl,
c. halo(C₁₋₅)alkoxy,
d. carboxy(C₁₋₅)alkyl,
e. hydroxy,
f. C₁₋₅ alkoxy,
g. hydroxy(C₁₋₅)alkyl,
h. NR⁵R⁶, wherein
R⁵ and R⁶ are independently hydrogen, fluoro(C₁₋₅)alkyl or C₁₋₅ alkyl,
i. phenyl(C₁₋₅)alkyl,
j. C₆₋₁₀ aryl,
k. heteroaryl,
l. heterocycle,
m. heterocycle(C₁₋₅)alkyl, and
n. C₃₋₆ cycloalkyl,
wherein said phenyl(C₁₋₅)alkyl, C₆₋₁₀ aryl, heteroaryl, heterocycle; heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
and, X is selected from the group consisting of hydrogen, halogen, halo(C₁₋₄)alkyl, halo(C₁₋₄)alkyl amino, halo(C₁₋₄)alkyl(C₁₋₄ alkyl)amino, and Sn(alkyl)₃;
provided that in each instance, said halogen is other than a radiolabeled halogen; and
if R⁴ is other than NR⁶R⁷ then R¹ is methylamino or dimethyl amino;
Formula VIII 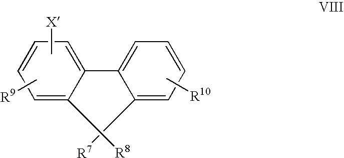
or a pharmaceutically acceptable salt thereof, wherein:
R⁹ and R¹⁰ are independently selected from the group consisting of:
a. hydrogen,
b. C₁₋₅ alkyl,
c. cyano,
d. trifluoromethyl,
e. nitro,
f. halogen,
g. hydroxy(C₁₋₅)alkyl,
h. halo(C₁₋₅)alkyl,
i. C₁₋₅ alkylthio,
j. halo(C₁₋₅)alkoxy,
k. carboxy(C₁₋₅)alkyl,
l. hydroxy,
m. C₁₋₅ alkoxy,
n. NR¹¹R¹², wherein
R¹¹ and R¹² are independently hydrogen, fluoro(C₁₋₅)alkyl or C₁₋₅ alkyl,
o. phenyl(C₁₋₅)alkyl,
p. C₆₋₁₀ aryl,
q. heteroaryl,
r. heterocycle,
s. heterocycle(C₁₋₅)alkyl, and
t. C₃₋₆ cycloalkyl,
wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
R⁷ and R⁸ are independently selected from the group consisting of hydrogen, hydroxy, hydroxy(C₁₋₅)alkyl, C₁₋₅ alkyl, C₁₋₅ alkoxy, halogen, carboxy(C₁₋₅)alkyl, trifluoromethyl, and halo(C₁₋₅)alkyl, phenyl(C₁₋₅)alkyl, C₃₋₆ cycloalkyl, heterocycle(C₁₋₅)alkyl, or R⁷ and R⁸ can be taken together to form a carbonyl, and
X′ is selected from the group consisting of hydrogen, halogen, halo(C₁₋₄)alkyl, halo(C₁₋₄)alkyl amino, halo(C₁₋₄)alkyl(C₁₋₄ alkyl)amino, and Sn(alkyl)₃;
provided that in each instance, said halogen is other than a radiolabeled halogen; 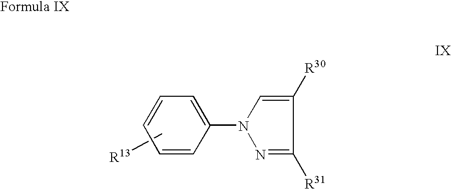
or a pharmaceutically acceptable salt thereof, wherein:
R¹³ is selected from the group consisting of:
a. C₁₋₅ alkyl,
b. cyano,
c. trifluoromethyl,
d. nitro,
e. halo(C₁₋₅)alkyl,
f. C₁₋₅ alkylthio,
g. hydroxy(C₁₋₅)alkyl,
h. halogen,
i. halo(C₁₋₅)alkoxy,
j. carboxy(C₁₋₅)alkyl,
k. hydroxy,
l. C₁₋₅ alkoxy,
m. NR¹⁴R¹⁵, wherein
R¹⁴ and R¹⁵ are independently hydrogen, halo(C₁₋₅)alkyl or C₁₋₅ alkyl,
n. phenyl(C₁₋₅)alkyl,
o. C₆₋₁₀ aryl,
p. heteroaryl,
q. heterocycle,
r. heterocycle(C₁₋₅)alkyl, and
s. C₃₋₆ cycloalkyl,
wherein said phenyl(C₁₋₅)alkyl, C₆₋₁₀ aryl, heteroaryl, heterocycle, heterocycle(C₁₋₅)alkyl or C₃₋₆ cycloalkyl is substituted with one of the following: C₁₋₅ alkylthio, C₁₋₅ alkylsulfonyl, methoxy, hydroxy, dimethylamino or methylamino,
and,
R³⁰ and R³¹ are selected from the group consisting of hydrogen, hydroxy, hydroxy(C₁₋₅)alkyl, C₁₋₅ alkyl, C₁₋₅ alkoxy, (C₁₋₅)alkyl carboxy, halogen, carboxy(C₁₋₅)alkyl, trifluoromethyl, halo(C₁₋₅)alkyl, phenyl(C₁₋₅)alkyl, C₃₋₆cycloalkyl and heterocycle(C₁₋₅)alkyl;
provided that in each instance, said halogen is other than a radiolabeled halogen; 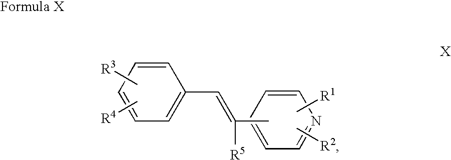
or a pharmaceutically acceptable salt thereof, wherein:
R⁵ is hydrogen or C₁₋₄ alkyl;
R¹, R² and R³, in each instance, is independently selected from the group consisting of hydrogen, hydroxy, halogen, C₁₋₄ alkyl, C₁₋₄ alkoxy, cyano, carboxy(C₁₋₅)alkyl, trifluoromethyl, nitro, methylamino, dimethylamino, halo(C₁₋₄)alkyl, and formyl;
R⁴ is selected from the group consisting of:
a. C₁₋₄ alkylthio,
b. C₁₋₄ alkylsulfonyl,
c. hydroxy,
d. C₁₋₄ alkoxy,
e. NR⁶R⁷, wherein
R⁶ and R⁷ are independently hydrogen or C₁₋₄ alkyl,
f. phenyl(C₁₋₄)alkyl,
g. C₆₋₁₀ aryl,
h. heteroaryl,
i. heterocycle,
j. heterocycle(C₁₋₄)alkyl, and
k. C₃₋₆ cycloalkyl,
wherein said phenyl(C₁₋₄)alkyl, C₆₋₁₀ aryl, heteroaryl, heterocycle, heterocycle(C₁₋₄)alkyl or C₃₋₆ cycloalkyl is substituted with one of the following: C₁₋₄ alkylthio, C₁₋₄ alkyl sulfonyl, methoxy, hydroxy, dimethylamino or methylamino;
or R³ and R⁴ are taken together to form an optionally substituted aryl or heteroaryl ring, wherein said ring is attached at adjacent carbons on the core pyridine ring;
provided that,
in each instance, said halogen is other than a radiolabeled halogen; and
if R⁴ is other than NR⁶R⁷, then R³ is methylamino or dimethyl amino.

2. The pharmaceutical composition of claim 1, having the following structure: 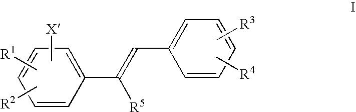
or a pharmaceutically acceptable salt thereof, wherein:
R¹, R², R³, R⁴, R⁵ and X′ are as described above.

3. The pharmaceutical composition of claim 2, wherein R⁴ is NR⁶R⁷, wherein R⁶ and R⁷ are independently hydrogen or C₁₋₄ alkyl.

4. The pharmaceutical composition of claim 3, wherein R¹, R², and R³ in each instance, is independently selected from the group consisting of hydrogen, halogen, C₁₋₄ alkyl, cyano, carboxy(C₁₋₅)alkyl, trifluoromethyl, nitro, methylamino, dimethylamino, halo(C₁₋₄)alkyl, and formyl.

5. The pharmaceutical composition of claim 4, wherein R⁵ is hydrogen.

6. The pharmaceutical composition of claim 5, wherein X′ is hydrogen, halogen or Sn(alkyl)₃.

7. The pharmaceutical composition of claim 1, wherein R² is hydroxy, trifluoromethyl or C₁₋₄ alkoxy.

8. The pharmaceutical composition of claim 1, wherein R³ and R⁴ are taken together to form an optionally substituted aryl or heteroaryl ring, wherein said ring is attached at adjacent carbons on the appropriate stilbene ring.

9. The pharmaceutical composition of claim 8, wherein X′ and R² are each hydrogen.

10. The pharmaceutical composition of claim 9, wherein said ring is an optionally substituted aryl ring.

11. The composition of claim 11, having the following structure: 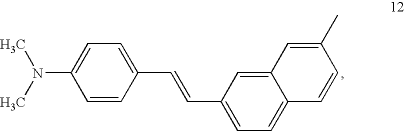
or a pharmaceutically acceptable salt thereof.

12. The pharmaceutical composition of claim 11, wherein R¹¹ is hydrogen and R¹² is NR¹³R¹⁴.

13. The pharmaceutical composition of claim 1, having the following structure: 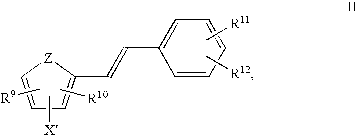
or a pharmaceutically acceptable salt thereof, wherein:
Z, R⁹, R¹⁰, R¹¹, R¹² and X′ are as described above.

14. The pharmaceutical composition of claim 1, having the following structure: 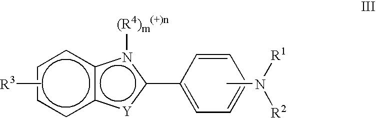
or a pharmaceutically acceptable salt thereof, wherein:
R¹, R², R³, R⁴, m and n are as described above.

15. The pharmaceutical composition of claim 14, wherein m is zero.

16. The pharmaceutical composition of claim 15, wherein R¹ and R² are each independently hydrogen or C₁₋₄ alkyl.

17. The pharmaceutical composition of claim 16, wherein R¹ and R² are each methyl.

18. The pharmaceutical composition of claim 17, wherein R³ is halogen, hydrogen, C₁₋₄ alkyl or Sn(alkyl)₃.

19. The composition of claim 18, wherein Y is O.

20. The composition of claim 19, having the following structure: 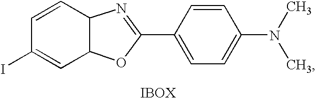
or a pharmaceutically acceptable salt thereof.

21. The composition of claim 18, wherein Y is S.

22. The composition of claim 21, having the following structure: 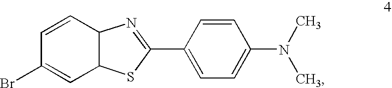
or a pharmaceutically acceptable salt thereof.

23. The pharmaceutical composition of claim 1, having the following structure: 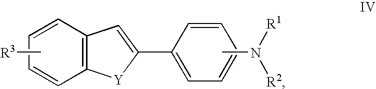
or a pharmaceutically acceptable salt thereof, wherein R¹, R², R³ and Y are as described above.

24. The pharmaceutical composition of claim 23, wherein R¹ and R² are each independently hydrogen or C₁₋₄ alkyl.

25. The pharmaceutical composition of claim 24, wherein R³ is halogen, hydrogen, C₁₋₄ alkyl or Sn(alkyl)₃.

26. The pharmaceutical composition of claim 25, wherein Y is O.

27. The pharmaceutical composition of claim 1, having the following structure: 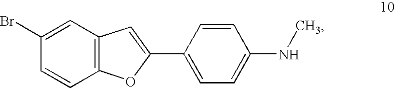
or a pharmaceutically acceptable salt thereof.

28. The pharmaceutical composition of claim 1, having the following structure: 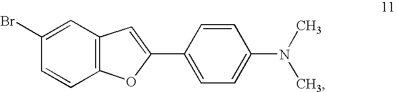
or a pharmaceutically acceptable salt thereof.

29. The pharmaceutical composition of claim 1, having the following structure: 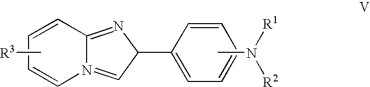
or a pharmaceutically acceptable salt thereof, wherein R¹, R² and R³ are as described above.

30. The pharmaceutical composition of claim 29, wherein R¹ and R² are each independently hydrogen or methyl.

31. The pharmaceutical composition of claim 30, wherein R³ is halogen, hydrogen, C₁₋₄ alkyl or Sn(alkyl)₃.

32. The pharmaceutical composition of claim 31, having the following structure: 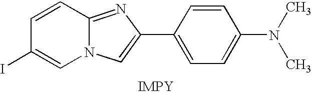
or a pharmaceutically acceptable salt thereof.

33. The pharmaceutical composition of claim 1, having the following structure: 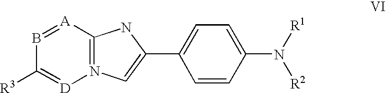
or a pharmaceutically acceptable salt thereof, wherein R¹, R², R³, A, B and D are as described above.

34. The pharmaceutical composition of claim 33, wherein R³ is halogen, hydrogen, C₁₋₄ alkyl or Sn(alkyl)₃.

35. The pharmaceutical composition of claim 1, having the following structure: 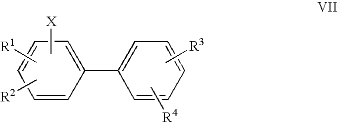
or a pharmaceutically acceptable salt thereof, wherein R¹, R², R³, R⁴ and X are as described above.

36. The pharmaceutical composition of claim 35, wherein R³ is hydrogen or halogen.

37. The pharmaceutical composition of claim 36, wherein R⁴ is hydroxy, C₁₋₅ alkoxy or hydroxy(C₁₋₅)alkyl.

38. The pharmaceutical composition of claim 37, wherein R² is hydrogen.

39. The pharmaceutical composition of claim 38, wherein X is hydrogen, halogen or Sn(alkyl)₃.

40. The pharmaceutical composition of claim 39, having the following structure: 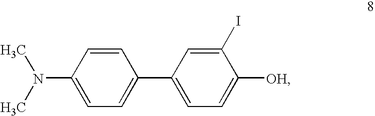
or a pharmaceutically acceptable salt thereof.

41. The pharmaceutical composition of claim 1, 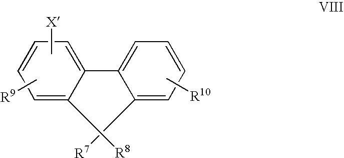
or a pharmaceutically acceptable salt thereof, wherein R⁷, R⁸, R⁹, R¹⁰ and X′ are as described above.

42. The pharmaceutical composition of claim 41, wherein R⁷ and R⁸ are each independently hydrogen, hydroxy, or R⁷ and R⁸ are taken together to form a carbonyl.

43. The pharmaceutical composition of claim 42, wherein X′ is hydrogen, halogen or Sn(alkyl)₃.

44. The pharmaceutical composition of claim 43, wherein R⁹ is hydrogen or NR¹¹R¹², wherein R¹¹ and R¹² are as described above.

45. The pharmaceutical composition of claim 44, having the following structure: 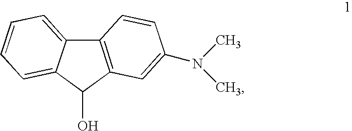
or a pharmaceutically acceptable salt thereof.

46. The pharmaceutical composition of claim 45, having the following structure: 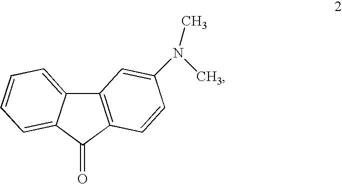
or a pharmaceutically acceptable salt thereof.

47. The pharmaceutical composition of claim 44, having the following structure: 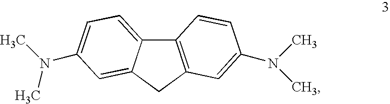
or a pharmaceutically acceptable salt thereof.

48. The pharmaceutical composition of claim 44, having the following structure: 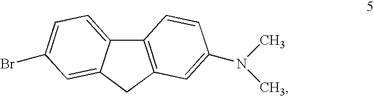
or a pharmaceutically acceptable salt thereof.

49. The pharmaceutical composition of claim 44, having the following structure: 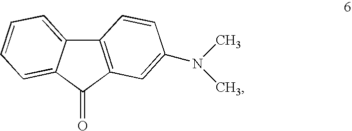
or a pharmaceutically acceptable salt thereof.

50. The pharmaceutical composition of claim 1, having the following structure: 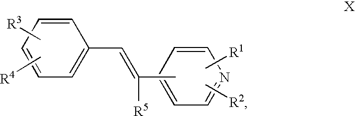
or a pharmaceutically acceptable salt thereof, wherein:
R¹, R², R³, R⁴ and R⁵ are as described as above.

51. The pharmaceutical composition of claim 50, wherein R¹ R² and R³ are each independently hydrogen or halogen.

52. The pharmaceutical composition of claim 51, having the following structure: 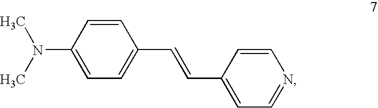
or a pharmaceutically acceptable salt thereof.

53. The pharmaceutical composition of claim 1, further comprising one or more other drugs that treat, prevent, control, ameliorate, or reduce the risk of side effects or toxicity of the compounds of claim 1.

54. The pharmaceutical composition of claim 53, wherein said one or more other drugs is selected from the group consisting of anti-Alzheimer's agents, HMG-CoA reductase inhibitors, non-steroidal anti-inflammatory drugs (NSAIDs), vitamin E, anti-amyloid antibodies, humanized monoclonal antibodies, CB-1 receptor antagonists, CB-1 receptor inverse agonists, antibiotics, N-methyl-D-aspartate (NMDA) receptor antagonists, cholinesterase inhibitors, growth hormone secretagogues, histamine H₃ antagonists, 2-amino-3-(3-hydroxy-5-methyl-4-isoxazolyl)propionic acid (AMPA) receptor agonists, phosphodiesterase (PDE) IV inhibitors, GABA_{A} (gama-aminobutyric acid) inverse agonists, and neuronal nicotinic agonists.

55. The pharmaceutical composition of claim 54, wherein said one or more other drugs is selected from the group consisting of beta-secretase inhibitors, gamma-secretase inhibitors, ibuprofen, doxycycline, rifampin, memantine, galantamine, rivastigmine, donepezil, tacrine, ibutamoren, ibutamoren mesylate and capromorelin.

56. A method for preventing, controlling, ameliorating or reducing the risk of Alzheimer's disease in a patient in need thereof comprising administering to the patient said pharmaceutical composition of claim 1, in an amount effective to preventing, controlling, ameliorating or reducing said risk of Alzheimer's disease.

57. The method of claim 56, further comprising administering said pharmaceutical composition of claim 1 with one or more other drugs that treat, prevent, control, ameliorate, or reduce the risk of side effects or toxicity of said pharmaceutical composition of claim 1.

58. The method of claim 57, wherein said one or more other drugs is administered contemporaneously or sequentially with said pharmaceutical composition of claim 1.

59. The method of claim 58, wherein said one or more other drugs is administered as part of a unit dosage form combination product, as a kit or treatment protocol, wherein said one or more other drugs are administered in separate dosage forms as part of a treatment regimen.

60. A method of inhibiting amyloid plaque aggregation in a mammal, comprising administering a composition of claim 1 in an amount effective to inhibit amyloid plaque aggregation.
